SEQUENCE LISTING (89 sequences)

SEQ ID NO: 1 - protein, 236 aa, Tachypleus tridentatus
VGGESMLRGVYQDKFYQGTYPQNKNDNWLARATLIGKGGWSNFKFLFLSPGGELYGVLNDKIYKGTPPTHDNDNWMGRAKKIGNGGWNQFQFLFFDPNGYLYAVSKDKLYKASPPQSDTDNWIARATEIGSGGWSGFKFLFFHPNGYLYAVHGQQFYKALPPVSNQDNWLARATKIGQGGWDTFKFLFFSSVGTLFGVQGGKFYEDYPPSYAHDNWLARAKLIGNGGWDDFRFLFF

SEQ ID NO: 2 - protein, 330 aa, Lycopersicon esculentum
MPLSSPPPHEPSPPPPYPRCGMGGGDGKCKSNECCSIWSWCGTTESFCAPQNCQSQCPHTPSPPPPSPPPPSPSPPPPSPPPPSPSPPPPTPSPPPPAPSPPPPSPPPPSPSPPPPSPPPPSPSPPPPTPSPPPPSPSPPPPTPSPPPPAPSPPPPYPRCGMGGGGGKCKSNECCSIWSWCGTTESYCAPQNCQSQCPHTPSPSPPPTPPPPYPRCGMGGGGGKCKSNECCSIWSWCGTTESYCAPQNCQSQCPHTPSPSPPPTPPPPYPRCGMGGGGGKCKSNECCSIWSWCGTTESYCAPQNCQSQCKKNIISSVMNPMNVTYGIESF

SEQ ID NO: 3 - protein, 267 aa, Kappaphycus alvarezii
GRYTVQNQWGGSSAPWNDAGLWILGSRSNQNVMAIDVNSSDGGANLNGTMTYSGEGPIGFKGARRGESNVYDVENQWGGSSAPWHAGGQFVIGSRSGQGVLAVNITSSDGGKTLTGTMTYEREGPIGFKGTQSGGDTYNVENQWGGSSAPWNKAGIWALGDRSGQAMIAMDVSSSDGGKTLEGTMQYKGEGPIGFRGKLSGANNYSVENQWGGSSAPWNKAGDWLIGDRHNQNITAVKVSSDNDGKNLDGTCTYESEGPIGFKGVAS

SEQ ID NO: 4 - protein, 107 aa, Bryopsis corticulans
ADVGTVTVKGLGDRGSCPVKPWTSAQTNAREKVVSVKFDIPYSSTPKVALSLSGMDMDKKYNTRINTSVENLTNEGFDLKVGVWCNTYAYKLVVTYIAVPAPYQATA

SEQ ID NO: 5 - protein, 101 aa, Helix pomatia
RVQSGKIDCGNDAGWAKVPSDDPGRDNTRELAKNITFASPYCRPPVVLLSITQLDVEQSQNLRVIARLYSVSPTGFKASCYTWHNTKVYSMSISWISIENY

SEQ ID NO: 6 - protein, 301 aa, Solanum tuberosum
NELSLPFHLPINETIGLEVFQGINNASPPSPSPLPYPQCGMKKGGGKCIKTGECCSIWGWCGTTNAYCSPGYCQKQCPGPYPEGRCGWQANGKSCPTGTGQCCSNGGWCGTTSDYCASKNCQSQCKLPSPPPPPPPPSPPPPSPPSPPPPSPPPPPPPSPPPPSPPPPSPSPPPPPASPPPPPPALPYPQCGIKKGGGKCIKTGECCSIWGWCGTTNAYCSPGYCQKQCPGPYPEGRCGWQANGKSCPTGTGHCCSNAGWCGTTSDYCAPVNCQAQCNTTTLTSPIKNRMRGIESFMLNVV

SEQ ID NO: 7 - protein, 161 aa, Boodlea coacta
GAFKAISGESGKYLSHAFAKIWLQNGYQGLGEAWDIQYFGNNVIAMHAKGGEEGTTLSHAFGKIWLQKGVQGDGEKWKYEWVGNGYVFHCLGGETGYTLSHAFSKVWLQKGHQGDGEVWLYENKGEGVVGNSDSLEEAPEVLFQAQGIPEGLEDPPKLTIS

SEQ ID NO: 8 - protein, 162 aa, Boodlea coacta
GAFKAIGGESGKYLSHAFAKIWLQNGYQGLGEAWDIEYFGNNVIAMHAKGGESGTTLSHAFGKIWLQNGVQGDGEKWKYEWVGHGYVFHCLGGESGYTLSHAFNKIWLQKGHQGDGEVWLYENKGEGVVGSSNNLEEAPEVLFQKPQGSPDGPEGTPKLPIS

SEQ ID NO: 9 - protein, 125 aa, Boodlea coacta
GAFKAIGGESGKYLSHAFAKIWLQNGYQGLGEAWDIEYFGNNVIAMHAKGGESGTTLSHAFGKIWLQNGVQGDGEKWKYEWVGHGYVFHCLGGESGYTLSHAFNKIWLQKGHQGDGEVWLYENKG

SEQ ID NO: 10 - protein, 260 aa, Ulva limnetica
ANQDAGAIMASPVEQDRCLTMCPTCCLDKDSSDFGLCLESADSLSVANCFVGDPMADPVTEVDAGVTDVSADCNTDSAFQLLQNSWSWMTSLPSVFQMSIQAPGSSRRYFAAHVYYGSDNRDSASTFVCSHDTGSKKAILLTVPAAQGGVKLMVLENRDTTGKGKTEGRYLSAHNYYSSDRVFSGQIRLMIHTDPNKAAVWRYEGNKLKVVQNLDATAPDTAGWYLTAPSEARRDSASTFLTVQSDASKAALVIAETVTA

SEQ ID NO: 11 - protein, 88 aa, Urtica dioica
RCGSQGGGGTCPALWCCSIWGWCGDSEPYCGRTCENKCWSGERSDHRCGAAVGNPPCGQDRCCSVHGWCGGGNDYCSGSKCQYRCSSS

SEQ ID NO: 12 - protein, 90 aa, Hypnea japonica
FGPGCGPSTFSCTSPQKILPGSSVSFPSGYSSIYLTTESGSASVYLDRPDGFWVGGADSRGCSNFGGFNGNGDSKVGNWGDVPVAAWACN

SEQ ID NO: 13 - protein, 112 aa, Bryopsis maxima
SDVGVVEVKGLGDRGSCPVRPWTDAQTSAREKVFSIKFDIPYSSTPKVALSLSGMDMDKRYNTRLTTSVENLTNEGFDLKVGVWCNTYAYRVMVTYVAVPAPYQAAAPTMKH

SEQ ID NO: 14 - protein, 106 aa, Bryopsis maxima
TDVGNVEVRGLGDRSSCPVKPWTTAQTSAREKVISIKFDIPYSSTPKVALSLSGMDMDTKYNTRINTSVENLTNEGFDLKVGVWCNTYAYMLDVTYVVVPAPYTAG

SEQ ID NO: 15 - protein, 254 aa, Griffonia simplicifolia
ADTVCFTYPNFGNAVSDLILQGAATPDKGTVKLTATDSNGIPRTNSVGRVLFSSPIHLWEKSTGKEASFSTSFSFITKPSPKGGTIADGLTFFIAPPGTTIPSKIEGEYLGVLEPSTGNDPSKNQIVFCEFDLYKNGIDPSYTPHLGINVNQIKSEVTAPWNTTNVPTGSTAFVRITYDAPSKKLSVTLSYPDVSNSFRSTLSHTVSLKDKLPEWVSVGISGCSGLQVSLNNLLSWSFSSELKKVGTSFAITDM

SEQ ID NO: 16 - protein, 395 aa, Psathyrella velutina
QALPVPTRIPGVADLVGFGNGGVYIIRNSLLIQVVKVINNFGYDAGGWRVEKHVRLLADTTGDNQSDVVGFGENGVWISTNNGNNTFVDPPKMVLANFAYAAGGWRVEKHIRFMADLRKTGRADIVGFGDGGIYISRNNGGGQFAPAQLALNNFGYAQGWRLDRHLRFLADVTGDGLLDVVGFGENQVYIARNSSNGTFQPAQAVVNNFCIGAGGWTISAHPRVVADLTGDRKADILGFGVAGVYTSLNNGNGTFGAVNLVLKDFGVNSGWRVEKHVRCVSSLTNKKVGDIIGFGDAGVYVALNNGNGTFGPVKRVIDNFGYNQGWRVDKHPRFVVDLTGDGCADIVGFGENSVWACMNKGDGTFGPIMKLIDDMTVSKGWTLQKTVRYAANLYL

SEQ ID NO: 17 - protein, 186 aa, Triticum vulgare
QRCGEQGSGMECPNNLCCSQYGYCGMGGDYCGKGCQNGACWTSKRCGSQAGGKTCPNNHCCSQYGHCGFGAEYCGAGCQGGPCRADIKCGSQAGGKLCPNNLCCSQWGYCGLGSEFCGEGCQNGACSTDKPCGKDAGGRVCTNNYCCSKWGSCGIGPGYCGAGCQSGGCDGVFAEAIATNSTLLAE

SEQ ID NO: 18 - DNA, 23 bp, Artificial Sequence
  Artificially synthesized primer sequence
cgactggagcacgaggacactga

SEQ ID NO: 19 - DNA, 26 bp, Artificial Sequence
  Artificially synthesized primer sequence
ggacactgacatggactgaaggagta

SEQ ID NO: 20 - DNA, 25 bp, Artificial Sequence
  Artificially synthesized primer sequence
gctgtcaacgatacgctacgtaacg

SEQ ID NO: 21 - DNA, 23 bp, Artificial Sequence
  Artificially synthesized primer sequence
cgctacgtaacggcatgacagtg

SEQ ID NO: 22 - DNA, 28 bp, Artificial Sequence
  Artificially synthesized primer sequence
aycantayaaygtngaraaycartgggg

SEQ ID NO: 23 - DNA, 25 bp, Artificial Sequence
  Artificially synthesized primer sequence
aytgrttytcnacrttrtantgrtc

SEQ ID NO: 24 - DNA, 24 bp, Artificial Sequence
  Artificially synthesized primer sequence
atnggnccytcnccyttrtaytgc

SEQ ID NO: 25 - DNA, 24 bp, Artificial Sequence
  Artificially synthesized primer sequence
atagctgagtcaagttacaccaac

SEQ ID NO: 26 - DNA, 21 bp, Artificial Sequence
  Artificially synthesized primer sequence
agagggtgatcacgtttttac

SEQ ID NO: 27 - DNA, 23 bp, Artificial Sequence
  Artificially synthesized primer sequence
ccccggtcccccarnccyttnac

SEQ ID NO: 28 - DNA, 20 bp, Artificial Sequence
  Artificially synthesized primer sequence
cacctccgcttctactcctg

SEQ ID NO: 29 - DNA, 24 bp, Artificial Sequence
  Artificially synthesized primer sequence
atttgttgctattctctgcactgc

SEQ ID NO: 30 - DNA, 21 bp, Artificial Sequence
  Artificially synthesized primer sequence
caacgcactaacaagcgttac

SEQ ID NO: 31 - DNA, 21 bp, Artificial Sequence
  Artificially synthesized primer sequence
ctcnctggcntngntctgngc

SEQ ID NO: 32 - DNA, 19 bp, Artificial Sequence
  Artificially synthesized primer sequence
acggatacttctggctgca

SEQ ID NO: 33 - DNA, 20 bp, Artificial Sequence
  Artificially synthesized primer sequence
atccgatctacacttcgcga

SEQ ID NO: 34 - DNA, 804 bp, Kappaphycus alvarezii
  CDS (1)..(804)
ggacgttacacagttcagaaccaatgggggggatcctctgccccctggaatgatgccggcttgtggatccttggcagtcgtagcaaccagaatgtgatggctattgatgttaactccagcgatggaggtgccaacctgaacggtacgatgacctactccggtgaaggcccgattggattcaagggggcacgccgtggtgagtcaaatgtttacgatgtagagaaccagtggggaggatcttccgccccctggcacgctggaggccaattcgttatcgggtccaggtctggacagggagtgcttgccgtgaacattacttcttcagatggcggaaagacattgactggcaccatgacctatgagagagaaggtccaattggattcaagggtactcagtctggcggtgacacttacaacgtagagaaccagtggggtgggtcctcggcgccgtggaacaaggcaggtatttgggcgctcggggatcgcagtgggcaagcaatgattgctatggatgtttcgtcctcggatggtggaaaaactctagaggggacgatgcagtacaaaggagagggaccgattggattccgtggcaagctgagcggtgccaacaactacagtgtcgagaaccagtggggcggttcctctgctccttggaacaaggctggagactggttgattggagaccgccacaatcaaaacataaccgcagtcaaggtgtcatctgacaatgatggaaagaacctcgacggcacatgcacgtacgagagcgagggtcccattggtttcaagggagtcgcttcctaa

SEQ ID NO: 35 - DNA, 324 bp, Bryopsis corticulans
  CDS (1)..(324)
gctgatgtcggcaccgttacagtcaaagggttgggagacaggggatcctgccccgtgaaaccctggacctctgcacagaccaacgccagagagaaggttgtctccgtcaaattcgacattccatactcctccactcccaaggtggctttgtctctctccggtatggatatggacaagaagtacaacaccaggatcaacacctctgtggagaacctcacgaacgagggattcgacttgaaggtcggagtgtggtgcaacacgtacgcctacaaactcgtggtgacgtacattgccgtccccgccccctaccaagcaacagcttga

SEQ ID NO: 36 - DNA, 339 bp, Bryopsis maxima
  CDS (1)..(339)
tctgacgtcggggtcgttgaagtcaaaggattgggagacaggggatcctgccccgtgagaccctggaccgatgcacagacctccgccagagagaaggttttctccatcaagttcgacattccatactcctccactcccaaggtggctttgtctctctccggtatggatatggacaagaggtacaacaccaggctcaccacctctgtggagaacctcaccaacgagggattcgacttgaaggtcggagtgtggtgcaacacgtacgcgtacagggtcatggtgacttatgttgctgtccccgccccctaccaagccgccgctcctacgatgaaacactga

SEQ ID NO: 37 - DNA, 321 bp, Bryopsis maxima
  CDS (1)..(321)
actgacgttgggaacgttgaagtcagagggttgggagacaggtcgtcctgccccgtgaaaccctggaccactgcacagacctccgccagggagaaggttatttccatcaaattcgatattccatactcctccactcccaaggtcgccttgtctctctccggtatggatatggacaccaagtacaacaccaggatcaacacctctgtggagaacctcaccaacgagggattcgacttgaaagtcggagtgtggtgcaacacgtacgcctacatgctcgacgtgacctacgttgtggtccccgccccatacactgcgggatga

SEQ ID NO: 38 - protein, 84 aa, Codium barbatum
ISHGIKNDCGVPVTCKVSTSYGSSKYSMSPGQSLYRSYTNQKRACVYCWTGTDEKFHTTYICSEVNGSLTNVCSYAAWKSCSNT

SEQ ID NO: 39 - DNA, 318 bp, Codium barbatum
  CDS (1)..(318)
atgtacaggtgtgcaatcgtgttggtagctctgttgattggagctgaagctatcagtcacggcattaaaaatgattgtggtgtccctgttacttgcaaggtttctacctcatatggtagcagtaagtacagcatgtcacccgggcaatcactgtacaggagttataccaaccaaaagagggcatgcgtgtattgttggactgggactgatgaaaagtttcatacgacatacatttgtagtgaagttaatggaagtcttacaaatgtgtgttcgtatgctgcttggaagtcctgcagtaataccaacatggacacatga

SEQ ID NO: 40 - protein, 106 aa, Bryopsis corticulans
TDVGSVEVRGLGDRSSCPVKPWTTAQTSAREKVISIRFDIPYSSTPKVALSLSGMDMDTKYNTRINTSVENLTNEGFDLKVGVWCNTYAYMLDVTYVVVPAPYAAG

SEQ ID NO: 41 - DNA, 378 bp, Bryopsis corticulans
  CDS (1)..(378)
atggctgtcacaaagatcgctgttttgttgctcttggctgccgttggcgcctacgccactgacgtcgggagcgttgaggtcagagggttgggagacaggtcatcttgccccgtgaaaccctggaccactgcacagacatccgccagggagaaggttatttccatcagattcgacatcccatactcctccactcccaaggtcgctttgtctctctccggtatggatatggacaccaagtacaacaccaggatcaacacctctgtggagaacctcaccaacgagggattcgacttgaaagtcggtgtgtggtgcaacacctacgcctatatgctcgacgtgacctacgttgtggtccccgccccatacgctgcgggatga

SEQ ID NO: 42 - protein, 108 aa, Codium fragile
FQTGIVQESGFGDESECPVRPWTTAQSEARERFIPIVFDKPYTATPDIVLSFVRQDVKYGSVVGTSLRLLTEARDVTPEGFTLYIGTWCCTYVYSAYVQWLAVPSVYA

SEQ ID NO: 43 - DNA, 387 bp, Codium fragile
  CDS (1)..(387)
atggcctccgctcagtgcactctcttcctgcttctggcgctcctgggcctcagtcaagccttccaaacggggatcgttcaagagtcaggctttggagatgagtccgaatgcccagtgagaccttggaccaccgcccagagcgaagccagggagcgctttatcccaattgtttttgacaagccgtacactgccaccccagatattgttctcagcttcgttcgtcaggatgttaagtatgacagtgtagttggaaccagcctcaggctgttgacggaggcaagagacgtgactcctgaaggctttactctgtacattggcacctggtgctacacctatgtgtacagcgcctatgtgcagtggctcgcagtaccttctgtttatgcttga

SEQ ID NO: 44 - protein, 108 aa, Codium fragile
FQTGIVKESGIGDESECPVRPWTTAQSEARERFIPIVFDKPYTATPDIVLSFLRQDVKYDSVVGTSIRLETEARDVTPEGFTLYVGTWCYTYVFSVDVQWLAVPSVYA

SEQ ID NO: 45 - DNA, 387 bp, Codium fragile
  CDS (1)..(387)
atggcctccgctcagtgcactctcttcctgcttctggcgctcctgggcctcagtcaagccttccaaacggggattgttaaagagtcaggcattggagatgagtccgggtgcccagtaagaccttggaccaccgcccagagcgaagccagggagcgttttatcccaatcgttttcgacaagccttacactgccaccccagatattgttctcagctttcttcgtcaggatgttaagtatgatagtacttttggaaccagcatcagactggagacagaggcaagggatgttactcctgaaggcttcaccctctatgttggtacctggtgctacacctatgtgtacagcgtcgatgtgcagtggcttgcagtaccatcagtttatgcttga

SEQ ID NO: 46 - protein, 149 aa, Codium latum
ALHVTLTADTGKLLSRCRNCGPGAYPDSATVHQSSRSPSSTWEMTIVGDDKIMLKSNNGKYLARCNGCWSGGAYPDSAFVHATSQQSYSTWTVVGHADGKISLKSDTGKYLARCNGCVPGGAYPDSAFVHISDPNLPYAKWAVTYVSST

SEQ ID NO: 47 - DNA, 510 bp, Codium latum
  CDS (1)..(510)
atggtgaaggctattgcgatggtcgtgacgtttgccctgctggcggtggccgccagggcagccctgcacgtgacgctgactgcagacaccggcaagctgctgtctcgctgccgcaactgtgggccaggagcctaccctgacagcgcaacggtgcaccagagctcgcggtcaccttccagtacttgggagatgactattgtgggtgatgacaagatcatgctcaaatccaacaacggcaagtatttggccagatgcaacggctgctggtcaggtggagcatacccagactctgcatttgtgcatgccacaagccagcagtcgtactctacctggacagtggtgggacacgcagacggcaagatctccctcaagagtgacactggcaaatacttggctagatgcaatggttgtgtacccggtggtgcctatcctgattctgcgtttgttcatatctctgatcccaacctgccttatgccaaatgggctgtgacctacgtcagcagcacctaa

SEQ ID NO: 48 - protein, 267 aa, Meristotheca papulosa
GRYTVQNQWGGSSAPWNDAGLYVLGSRANQNVVAIEVSSNDGGANLTGTMTYSGEGPIGFKGARRGQSNVYEVKNQWGGSSAPWHDGGDFVIGSRSGQGVVGLDIKSSDNGKTLTGTMTYERKGPIGFKGTQSGGDSYNVENQWGGSSAPWNKAGIWALGDRAGQAMIKLEVTSSDGGSNLEGTMQYKGEGPIGFRAKLSGANNYSVENQWGGSSAPWNKAGDWLIGDRYNQNITAVNVASSDDGKNLEGTCTYAREGPIGFKGAAV

SEQ ID NO: 49 - DNA, 807 bp, Meristotheca papulosa
  CDS (1)..(807)
atgggacgctacacagttcagaaccagtggggaggctcttctgctccctggaatgatgccggtttgtacgtgcttggaagccgtgctaatcagaacgtggtggcaattgaagtcagctccaacgacggtggagccaacctcaccgggacaatgacgtactctggcgagggccccattgggttcaagggggcacgccggggccagtcaaacgtctatgaagtcaaaaaccagtggggcgggtcttcagctccctggcatgatggtggtgattttgtgatcgggtctaggtctgggcagggggtggtcggactggatatcaaatcgtctgacaacggaaagaccttgaccggaactatgacgtacgaaagaaagggtccaattgggttcaaggggacacagtctggtggtgactcatacaacgttgagaatcagtgggggggatcttcagctccctggaacaaggccggcatttgggctttgggggatcgcgcgggccaggcaatgatcaagcttgaggtgacctcttcagatggcggaagcaatttggaggggacgatgcagtacaagggtgagggaccgattggattcagggcaaagctaagtggtgcgaacaattacagtgtggagaatcagtggggcggatcttccgctccttggaacaaggcgggcgactggttgattggggaccgttataatcagaatattactgctgtgaatgtggcgtctagtgacgacgggaagaaccttgaaggcacatgtacgtatgcgcgtgagggcccgattggattcaagggtgccgctgtttaa

SEQ ID NO: 50 - protein, 267 aa, Meristotheca papulosa
GRYTVQNQWGGSSAPWNDAGLYVLGSRANQNVVAIEVSSNDGGANLTGTMTYSGEGPIGFKGARRGESNVYEVKNQWGGSSAPWHDGGDFVIGSRSGQGVVGLDIKSSDNGKTLTGTMTYEREGPIGFKGTQSGGDTYNVENQWGGSSAPWNKAGVWALGDRSSQAMIKLEVSSSDGGSTLEGTMQYKGEGPIGFRAKLASANNYSVENQWGGSSAPWNKAGDWLIGDRYNQNITAVNVSSSDDGKNLEGTCTYAREGPIGFKGSAV

SEQ ID NO: 51 - DNA, 807 bp, Merismopedia glauca
  CDS (1)..(807)
atgggacgctacacagttcagaaccagtggggaggctcttctgctccctggaatgatgccggcctgtatgtgcttggcagccgtgctaatcagaacgtggtggcaattgaagtcagctccaatgacggtggggctaacctcaccgggacaatgacatactctggcgagggccctattgggttcaagggggcacgccggggtgagtcaaacgtctatgaagtcaaaaaccagtggggcgggtcttcagctccctggcatgatggtggtgactttgtgatcgggtctaggtctgggcagggggtggtcggactggatatcaaatcgtctgacaacggaaagaccctgaccggaacgatgacatatgaaagggagggtccaattgggttcaaggggacgcagtctggtggtgacacatacaatgttgagaatcagtggggcggatcttctgctccctggaacaaggcaggcgtttgggccttgggggatcgctcgagccaggctatgatcaagctggaggtgagctcttcagatggcggaagcactttggagggtacgatgcagtacaagggtgagggacctattgggttcagggcaaagctggctagtgcgaacaactacagtgtagagaatcagtggggcggatcttccgcgccttggaacaaggccggtgactggttgatcggggaccgttacaaccagaacattactgccgtgaatgtgtcttctagcgatgacggaaagaatctggagggcacgtgcacgtatgcgcgtgagggccctattggatttaagggttctgctgtgtaa

SEQ ID NO: 52 - protein, 123 aa, Tachypleus tridentatus
TCVTDNTLDGTLMKHPSKPAVYQIIDGCRHWVPNPPTYNNLYKTWNCIKTNVLVEHICNCDSLSNGAELIKGSGPAVYLLSNGVKRHIANPDTFNAFCFDWNKIKTYSDIVINNISTGSVIER

SEQ ID NO: 53 - protein, 132 aa, Oscillatoria agardhii
ALYNVENQWGGSSAPWNEGGQWEIGSRSDQNVVAINVESGDDGQTLNGTMTYAGEGPIGFRATLLGNNSYEVENQWGGDSAPWHSGGNWILGSRENQNVVAINVESGDDGQTLNGTMTYAGEGPIGFKGTTL

SEQ ID NO: 54 - protein, 251 aa, Arachis hypogaea
SAETVSFNFNSFSEGNPAINFQGDVTVLSNGNIQLTNLNKVNSVGRVLYAMPVRIWSSATGNVASFLTSFSFEMKDIKDYDPADGIIFFIAPEDTQIPAGSIGGGTLGVSDTKGAGHFVGVEFDTYSNSEYNDPPTDHVGIDVNSVDSVKTVPWNSVSGAVVKVTVIYDSSTKTLSVAVTNDNGDITTIAQVVDLKAKLPERVKFGFSASGSLGGRQIHLIRSWSFTSTLITTTRRSIDNNEKKIMNMASA

SEQ ID NO: 55 - protein, 160 aa, Agrocybe cylindracea
TTSAVNIYNISAGASVDLAAPVTTGDIVTFFSSALNLSAGAGSPNNTALNLLSENGAYLLHIAFRLQENVIVFNSRQPNAPWLVEQRVSNVANQFIGSGGKAMVTVFDHGDKYQVVINEKTVIQYTKQISGTTSSLSYNSTEGTSIFSTVVEAVTYTGLA

SEQ ID NO: 56 - protein, 293 aa, Marasmium areades
MSLRRGIYHIENAGVPSAIDLKDGSSSDGTPIVGWQFTPDTINWHQLWLAEPIPNVADTFTLCNLFSGTYMDLYNGSSEAGTAVNGWQGTAFTTNPHQLWTIKKSSDGTSYKIQNYGSKTFVDLVNGDSSDGAKIAGWTGTWDEGNPHQKWYFNRMSVSSAEAQAAIARNPHIHGTYRGYILDGEYLVLPNATFTQIWKDSGLPGSKWREQIYDCDDFAIAMKAAVGKWGADSWKANGFAIFCGVMLGVNKAGDAAHAYNFTLTKDHADIVFFEPQNGGYLNDIGYDSYMAFY

SEQ ID NO: 57 - protein, 144 aa, Axinella polypoides
TSLCASKQRYLVINLQLGLFLTVKEPSGYSEATLESFNSGTNQLFCLIPEGNRYFIAFDNDDYDTVLDVEFAQDVAGARVIAYTKKASNDDNQLWGLVPLPETPGIIATALPSSNVITGTGIGESMEMQPEDPDLNQVFGFLKY

SEQ ID NO: 58 - protein, 101 aa, Nostoc ellipsosporum
LGKFSQTCYNSAIQGSVLTSTCERTNGGYNTSSIDLNSVIENVDGSLKWQPSNFIETCRNTQLAGSSELAAECKTRAQQFVSTKINLDDHIANIDGTLKYE

SEQ ID NO: 59 - protein, 130 aa, Polygonatum multiflorum
NSLTSPNSLPSGHSLNTGSYRLIMQADCNLVVYDSGKPVWATNTGGLARDCRLTMHNNGNLVIYDRSNSVIWQTNTNEKEDHYVLVLQQDRNVVIYGPAVWATGSGPAVGLTLVPHNVTAIVHARAMLNE

SEQ ID NO: 60 - protein, 109 aa, Allium sativum
RNLLTNGEGLYAGQSLDVEPYHFIMQEDCNLVLYDHSTSVWASNTGILGKKGCKAVLQSDGNFVVYDAEGRSLWASHSVRGNGNYVLVLQEDGNVVIYGSDIWSTGTYK

SEQ ID NO: 61 - protein, 249 aa, Ulex europaeus
NLSDDLSFNFDKFVPNQKNIIFQGDASVSTKGVLEVTKVSKPTTRSIGRALYAAPIQIWDSITGKVASFATSFSFVVKDEPDEKIDGVDGLAFFLAPANSQIPSGSSAGMFGLFCSSNDSKSSNQIIAVEFDSYFGKTYNPWDPDFKHIGIDVNSIKSIKTVKDDWRNGEVADVVITYRAPTKSLTVSLSYPSDGTSNIVTASSVDLKAILPEWVSVGFSGGVGNAAKFDHDVLSWYFTSNLEANQSQT

SEQ ID NO: 62 - protein, 91 aa, Ralstonia solanacearum
MSSVQTAATSWGTVPSIRVYTANNGKITERCWDGKGWYTGAFNEPGDNVSVTSWLVGSAIHIRVYASSGTTTTEWCWDGNGWTKGAYTSTN

SEQ ID NO: 63 - protein, 227 aa, Cicer arietinum
TKTGYINAAFRSSRNNEAYLFINDKYVLLDYAPGTSNDKVLYGPSFVRDGYKSLAKTIFGTYGIDCSFDTEYNEAFIFYENFCARIDYAPHSDKDKIISGPKKIADMFPFFKGTVFENGIDAAFRSTKGKEVYLFKGDKYARIDYLTNRLVQNIKSISDGFPCLRGTIFEAGMDSAFASHKTNEAYLFKGEYYARINFTPGSTNDIMGGVKKTLDYWPSLRGIIPLE

SEQ ID NO: 64 - protein, 141 aa, Cepaea hortensis
QIVEPTVAFTAVLDKNLVLENGDTLIPNKILINYGGGYNDKTGIFTAPKSGIYHLGVHAQTSLQSNLWLALYHNDNYVFSIYGRQTEYSDGGANAAILPLKKGDKVHVKARDKSSLLGRPDNIYTTFTGFRLGPLREDDSE

SEQ ID NO: 65 - protein, 250 aa, Laburnum alpinum
LNELSFNFDKFVPNQNNILFQGVASVSTTGVLQVTKVTNTGIKRALYAAPIHAWDDDSETGKVASFATSFSFVVKEPPIQSRKADGVDGLAFFLAPANSQIPSGSSAGMFGLFCSSDYNSSNQIIAVEFDTYFGKAYNPWDPDFKHIGVDVNSIKSIKTVKWDWRNGDVANVVITYRAPTKSLTVSLSYPSDQTSNIVTASVDLKAILPEWVSVGFSAGVGNAAKFNHDILSWYFTSNLEPNNPAVNQAQ

SEQ ID NO: 66 - protein, 253 aa, Dolichos biflorus
ANIQSFSFKNFNSPSFILQGDATVSSGKLQLTKVKENGIPTPSSLGRAFYSSPIQIYDKSTGAVASWATSFTVKISAPSKASFADGIAFALVPVGSEPRRNGGYLGVFDSDVYNNSAQTVAVEFDTLSNSGWDPSMKHIGIDVNSIKSIATVSWDLANGENAEILITYNAATSLLVASLVHPSRRTSYILSERVDITNELPEYVSVGFSATTGLSEGYIETHDVLSWSFASKLPDDSTAEPLDLASYLVRNVL

SEQ ID NO: 67 - protein, 232 aa, Tachypleus tridentatus
EWTHINGKLSHLTVTPRFVWGVNNVHDIFRCTRPCTGSNWIKVEGSLKQIDADDHEVWGVNSNDNIYKRPVDGNGSWIQIKGGLKHVSASGYGYIWGVNSKDQIFKCPKPCNGEWELVDGSLKQVDGGRDLVYGVTQNDEIFRRPVDGSGVWVNIPGKLKHISGSGSWEVFGVNCNDQIFRCKKPCSGQWVRLSGYLKQCDASGDSLLGVNSNDDIFESVPASKSCWMNPFL

SEQ ID NO: 68 - protein, 253 aa, Phaseolus lunatus
AELFFNFQTFNAANLILQGNASVSSSGQLRLTEVKSNGEPKVASLGRAFYSAPIQIWDSTTGKVASFATAFTFNILAPILSNSPDGLAFALVPVGSQPKFNGGFLGLFQNVTYDPTAQTVAVEFDTCHNLDWDPKGSHIGIDVNSIKSIKTVPWSLLNGHNAKVLITYDSSTKLLVASLVYPSGSTSYIISEKVELKSVLPEWVNIGFSATSGLNKGNVEAHDVLSWSFASKLSDGTPCEDLSLANIVLNKIL

SEQ ID NO: 69 - protein, 150 aa, Ulva pertusa
GGITNTDNWETFAGLPLTGAIKVNDGNSVVHISAYFPEDRRGKYSYYAATSDELQKTVVFLFVVEDDGLLLQAVKNNAHYPVTNGMYLASHRYYPKDSKYEGMVRLMVHADPAKAVIWEFVTVGGKQYLKVKENRDYTALQIPRHHPRPG

SEQ ID NO: 70 - protein, 262 aa, Bauhinia purpurea
TSSTLTGFTFPNFWSNTQENGTEIIFLGNATYTPGALRLTRIGEDGIPLKSNAGQASYSRPVFLWDSTGHVASFYTSFSFIVRSIDVPHITADGFAFFLAPVDSSVKDYGGCLGLFRYKTATDPSKNQVVAVEFDTWPNTEWSDLRYPHIGINVNSTVSVATTRWDNDDAYVTKSTAHITYDATSKIITVLLTYDNGRHYQLSHVVDLPKILPERVRIGFSGGTGFNETQYILSWSFTSTLNSTKISALTQKLRSSASYSSM

SEQ ID NO: 71 - protein, 141 aa, Musa acuminata
MNGAIKVGAWGGNGGSAFDMGPAYRIISVKIFSGDVVDGVDVTFTYYGKTETRHYGGSGGTPHEIVLQEGEYLVGMAGEVANYHGAVVLGKLGFSTNKKAYGPFGNTGGTPFSLPIAAGKISGFFGRGGKFLDAIGVYLEP

SEQ ID NO: 72 - protein, 114 aa, Flammulina velutipes
SATSLTFQLAYLVKKIDFDYTPNWGRGTPSSYIDNLTFPKVLTDKKYSYRVVVNGSDLGVESNFAVTPSGGQTINFLQYNKGYGVADTKTIQVFVVIPDTGNSEEYIIAEWKKT

SEQ ID NO: 73 - protein, 146 aa, Meristotheca papulosa
  MISC_FEATURE (1)..(1): Xaa stands for Pyroglutamic acid
XTGSCNTFQRSMMFGDPTGGAFFDDSCPLYVLSPRITRIEISVGGVVDQLIVTYSDGTRVSHGQPGSRRYTVDLGSNEYITSVFGRSGDLVDQIAFTSNIRTYGPFGSNGGSPYSVDFGGCALLYLFGRAGRSMDAIGFGYGPLSL

SEQ ID NO: 74 - DNA, 20 bp, Artificial Sequence
  Artificially synthesized primer sequence
acncayggnathaaraayga

SEQ ID NO: 75 - DNA, 20 bp, Artificial Sequence
  Artificially synthesized primer sequence
aaygaytgyggngtnccngt

SEQ ID NO: 76 - DNA, 20 bp, Artificial Sequence
  Artificially synthesized primer sequence
tccaagcagcatacgaacac

SEQ ID NO: 77 - DNA, 20 bp, Artificial Sequence
  Artificially synthesized primer sequence
tcatcagtcccagtccaaca

SEQ ID NO: 78 - DNA, 20 bp, Artificial Sequence
  Artificially synthesized primer sequence
cgcacggaaagaaaaaccgt

SEQ ID NO: 79 - DNA, 19 bp, Artificial Sequence
  Artificially synthesized primer sequence
tcrtayttnacrtcytgnc

SEQ ID NO: 80 - DNA, 23 bp, Artificial Sequence
  Artificially synthesized primer sequence
tanggyttrtcraanacdatngg

SEQ ID NO: 81 - DNA, 20 bp, Artificial Sequence
  Artificially synthesized primer sequence
gggatcgttcaagagtcagg

SEQ ID NO: 82 - DNA, 19 bp, Artificial Sequence
  Artificially synthesized primer sequence
attgttaaagagtcaggca

SEQ ID NO: 83 - DNA, 20 bp, Artificial Sequence
  Artificially synthesized primer sequence
gcnytncaygtnacnytnac

SEQ ID NO: 84 - DNA, 20 bp, Artificial Sequence
  Artificially synthesized primer sequence
acnytnacngcngayacngg

SEQ ID NO: 85 - DNA, 25 bp, Artificial Sequence
  Artificially synthesized primer sequence
gttggaatttagattgttgacttac

SEQ ID NO: 86 - DNA, 20 bp, Artificial Sequence
  Artificially synthesized primer sequence
agaaccagtggggaggatct

SEQ ID NO: 87 - DNA, 25 bp, Artificial Sequence
  Artificially synthesized primer sequence
artayaarggngarggnccnathgg

SEQ ID NO: 88 - DNA, 20 bp, Artificial Sequence
  Artificially synthesized primer sequence
tgtgccttcaaggttcttcc

SEQ ID NO: 89 - DNA, 20 bp, Artificial Sequence
  Artificially synthesized primer sequence
tatgcgtcgaagtcaccaac

(19) United States
(12) Patent Application Publication
(10) Pub. No.: US 2014/0087395 A1
(43) Pub. Date: Mar. 27, 2014

(54) METHOD FOR DISTINGUISHING BETWEEN SPECIES WITHIN THE GENUS STAPHILOCOCCUS

(71) Applicants: Hideaki Takeuchi, Nagoya-shi (JP); Koji Imamura, Ina-shi (JP); Uichiro Yabe, Nagoya-shi (JP); Kanji Hori, Higashi-Hiroshima-shi (JP); Makoto Hirayama, Higashi-Hiroshima-shi (JP)

(72) Inventors: Hideaki Takeuchi, Nagoya-shi (JP); Koji Imamura, Ina-shi (JP); Uichiro Yabe, Nagoya-shi (JP); Kanji Hori, Higashi-Hiroshima-shi (JP); Makoto Hirayama, Higashi-Hiroshima-shi (JP)

(73) Assignees: HIROSHIMA UNIVERSITY, Higashi-Hiroshima-shi, Hiroshima (JP); GLYENCE CO., LTD., Nagoya-shi, Aichi (JP)

(21) Appl. No.: 14/008,923

(22) PCT Filed: Mar. 22, 2012
(86) PCT No.: PCT/JP2012/057396

§ 371 Date: Dec. 5, 2013

(30) Foreign Application Priority Data
Mar. 31, 2011 (JP) 2011-077076

(51) Int. Cl. G01N 33/569; C07K 14/405

(52) U.S. Cl. CPC G01N 33/56938; C07K 14/405
USPC 435/7.33; 530/396; 530/370; 536/23.7

(57) ABSTRACT

The object was to provide a method for distinguishing between species within the genus Staphylococcus; binding affinities between many types of lectins and bacteria belonging to the genus Staphylococcus were examined; and lectins of Tachylectin-2, LEL, KAA1, BCL11, CBA, HAA, HPA, STL, proBCA1, proBCA2, ULL, DSA, PWM, UDA, WFL, hypninA3, BCL11d, CFA1, CFA2, CLA, MPA1, MPA2, AC-avranin, algCSA, BML11b, BML11c, etc. were selected. Further, it was found that these lectins could be used to distinguish between species within the genus Staphylococcus.

### TECHNICAL FIELD

The present invention relates to a method for distinguishing between species within the genus Staphylococcus. More particularly, it relates to a method for distinguishing between species within the genus Staphylococcus based on binding affinity to a specific lectin as an indicator.

### BACKGROUND ART

The bacteria belonging to the genus Staphylococcus are resident bacteria in the skin or gastrointestinal tracts of humans and others. The majority of them is non-pathogenic and forms an indigenous microbial flora in the skin or the like to be responsible for a part of the role as a barrier which prevents the invasion of pathogens from the outside. Although Staphylococcus aureus is one type of the Staphylococci, it is a causative bacterium of food poisoning, various epidermal infections such as abscess, or infections that will be fatal such as pneumonia, meningitis, and septicemic disease. For this reason, since it is quite important from the standpoint of food hygiene as well as of medical sciences to distinguish Staphylococcus aureus as the causative bacterium for food poisoning and the like from other Staphylococci that form barriers to prevent the invasion of pathogens, there is a need to establish a convenient and rapid distinguishing method.

The test/determination methods that have been carried out thus far are centered on culture methods utilizing selection/separation media. Pre-culturing is conducted in a mannite salt medium or the like for 48 h and pure-culturing is conducted for 24 h to discriminate bacteria; and verification tests such as a coagulase test, glucose fermentation test and Gram's staining are conducted. Therefore, the number of days, three to four days, is needed to detect the bacteria. Thus, the detection of the causative bacterium is restricted to a post factum test after the incidence such as food poisoning, and there is a problem that the bacterium cannot be detected before the intake of a food contaminated with Staphylococcus aureus.

As for the test/determination methods other than culturing, there are known methods, including a method for detecting an exotoxin of Staphylococcus aureus or an antibody against the exotoxin of Staphylococcus aureus in a specimen through an enzyme immunological technique (Patent Literature 1), a method utilizing polystyrene latex particles sensitized with human fibrinogen or immunoglobulin G to cause agglutination by being reacted specifically to Protein A produced by Staphylococcus aureus (Patent Literature 2), and a method for detecting Staphylococcus aureus through the sandwich ELISA utilizing an antibody that is specifically reactive to Staphylococcus aureus or an antibody that is specifically reactive to Protein A (Patent Literature 3). However, these methods require enrichment culture/separate culture and have a drawback that the proliferation of bacteria cannot be monitored real-time, for example, at locations of food processing. Further, the distinguishing methods using antibodies find difficulties in distinguishing between species within the genus Staphylococcus.

In recent years, genetic assays are in wide use. A genetic assay is a test for isolation and identification relying on the differences in DNAs or RNAs that are inherent to bacteria. There are known methods, including Real Time PCR utilizing primers against rRNA genes of Staphylococcus aureus (Patent Literature 4) and LAMP utilizing primers against the gapR gene of Staphylococcus aureus (Patent Literature 5). Since PCR and LAMP do not require the enrichment culture/separate culture, they are advantageous in rapidness as compared with other methods. However, as the assay of plural species requires reaction solutions corresponding to the number of the species, the complexity increases in proportion to the number of the targeted species. Therefore, the development of more convenient detection methods has been wanted.

Incidentally, the surface of a bacterium is covered with sugar chains, and the surface sugar chains function as an important factor that is responsible for the interaction between the host and the bacterium, pathogenicity, the cellular interaction, or immunity. It is also known that the surface sugar chains of bacteria differ depending on a bacterium. For example, lipopolysaccharides referred to as “O antigen” are present on the surfaces of Gram negative bacteria, and because the O antigens differ depending on bacterial species, they are used for classification. Moreover, it is reported that surface sugar chains are different between Staphylococcus aureus and Staphylococcus haemolyticus which is a skin resident bacterium (Non-patent Literature 1). Accordingly, it is thought that if the surface sugar chains of bacteria can be rapidly analyzed, the detection and identification of bacteria, which is more convenient than the conventional techniques, is possible.

In fact, it is reported that the cell surface sugar chains of Escherichia coli can be analyzed by allowing fluorescently stained Escherichia coli to react with a lectin microarray (Non-patent Literature 2). Also, Lu et al. succeeded in detecting Escherichia coli 0157:H strain in a wide range of from 6×10¹ to 6.1×10⁹ cells/ml by combining ConA lectin and a magnetoelastic sensor (Non-patent Literature 3). Further, there is a report that lectins can be used to distinguish Staphylococcus aureus from bacteria other than the genius Staphylococcus. Specifically, according to Payne et al., four types of organism-derived lectins (lectins derived from Agaricus bisporus, Helix pomatia, Triticum vulgaris, and Canavalia ensiformis) are used to distinguish among Staphylococcus aureus, Escherichia coli, Listeria, and Salmonella (Non-patent Literature 4); and according to Shang et al., Mannan-binding lectin is used to distinguish among the genus Staphylococcus, Escherichia coli, and Klebsiella bacteria (Non-patent Literature 5).

Moreover, with respect to distinguishing within the genus Staphylococcus, Munoz et al. also carried out the typing of Staphylococcus aureus by using 32 kinds of commercially available lectins (Non-patent Literature 6). Jarlov et al. carried out the typing of Staphylococcus epidermidis with four kinds of lectins (Non-patent Literature 7). Further, according to Sandra et al., two kinds of lectins that recognized N-acetylglucosamine are used to distinguish between Staphylococci which are coaglase-positive and Staphylococci which are coaglase-negative (Non-patent Literature 8).

Thus, while according to findings in the past there are reports concerning the typing of the genus Staphylococcus and other bacteria or of strains within species belonging to the genus Staphylococcus, the methods for distinguishing between species within the genus Staphylococcus, particularly methods for distinguishing Staphylococcus aureus from other Staphylococci, have not been in practical use, which reflects the present situations.

### CITATION LIST

### Patent Literature

[PTL 1] Japanese Unexamined Patent Application Publication No. H06-88824
[PTL 2] Japanese Unexamined Patent Application Publication No. H02-502942
[PTL 3] Japanese Unexamined Patent Application Publication No. H09-211000
[PTL 4] Japanese Unexamined Patent Application Publication No. 2006-508669
[PTL 5] Japanese Unexamined Patent Application Publication No. 2007-189980

### Non-Patent Literature

[NPL 1] Sigrid Flahaut et al., J. Bacteriol., March 2008, Vol. 190, No. 5, pp. 1649-1657
[NPL 2] Ku-Lung Hsu et al., Nat. Chem. Biol., March 2006, Vol. 2, No. 3, pp. 153-157
[NPL 3] Qingzu Lu et al., Anal. Chem., July 2009, Vol. 81, No. 14, pp. 5846-5850
[NPL 4] Ku-Lung Hsu et al., J Appl Bacteriol., July 1992, Vol. 73, No. 1, pp. 41-52
[NPL 5] Shang Shi-qiang et al., J Zhejiang Univ Sci B., January 2005, Vol. 6, No. 1, pp. 53-56
[NPL 6]A. Munoz et al., J Med. Microbiol., May 1999, Vol. 48, No. 5, pp. 495-499
[NPL 7]J. O. Jarlov et al., J Med. Microbiol., September 1992, Vol. 37, No. 3, pp. 195-200
[NPL 8] Sandra K. et al., J Clin Microbiol., April 1982, Vol. 15, No. 4, pp. 547-553

### SUMMARY OF INVENTION

### Technical Problem

The present invention has been made in view of the above-described problems of the prior art technologies. An object of the present invention is to provide a method capable of distinguishing between species within the genus Staphylococcus, particularly a method capable of rapidly and conveniently distinguishing between species within the genus Staphylococcus.

### Solution to Problem

The present inventors have devoted themselves to keen studies for achieving the above object. As a result, the present inventors have examined binding affinities between many types of lectins and bacteria belonging to the genus Staphylococcus and have selected lectins that display differing binding affinities among species within the genus Staphylococcus (Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, algMPL, and algCSA). Further, the present inventors have found that these lectins can be used to distinguish between species within the genus Staphylococcus. Still further, the present inventors have found that these lectins can be used to distinguish between species within the genus Staphylococcus even at the stationary phase, or at the logarithmic growth phase, and also in foods; and thus the present inventors have come to the completion of the present invention.

More specifically, the present invention provides the followings.

<1> A method for distinguishing between species within the genus Staphylococcus based on binding affinity to at least one lectin as an indicator, the lectin being selected from the group consisting of Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, algMPL, and algCSA.
<2> An agent for distinguishing between species within the genus Staphylococcus, comprising at least one lectin selected from the group consisting of Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, algMPL, and algCSA.
<3> A kit for distinguishing between species within the genus Staphylococcus, comprising:
a substrate where there is immobilized at least one lectin selected from the group consisting of Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, algMPL, and algCSA; and
at least one reagent selected from the group consisting of:
(a) a reagent for detecting a specimen;
(b) a blocking reagent;
(c) a reagent for immobilizing the specimen; and
(d) a reagent for diluting the specimen.
<4> At least one lectin selected from the group consisting of (a) to (c) below:
(a) a lectin comprising the amino acid sequence as set forth in SEQ ID NO:3;
(b) a lectin comprising an amino acid sequence having a homology of 90% or more to the amino acid sequence as set forth in SEQ ID NO:3; and
(c) a lectin encoded by a DNA that hybridizes with a DNA comprising the base sequence as set forth in SEQ ID NO:34 under stringent conditions.
<5> At least one lectin selected from the group consisting of (a) to (c) below:
(a) a lectin comprising the amino acid sequence as set forth in SEQ ID NO:4;
(b) a lectin comprising an amino acid sequence having a homology of 90% or more to the amino acid sequence as set forth in SEQ ID NO:4; and
(c) a lectin encoded by a DNA that hybridizes with a DNA comprising the base sequence as set forth in SEQ ID NO:35 under stringent conditions.
<6> At least one lectin selected from the group consisting of (a) to (c) below:
(a) a lectin comprising the amino acid sequence as set forth in SEQ ID NO:13;
(b) a lectin comprising an amino acid sequence having a homology of 90% or more to the amino acid sequence as set forth in SEQ ID NO:13; and
(c) a lectin encoded by a DNA that hybridizes with a DNA comprising the base sequence as set forth in SEQ ID NO:36 under stringent conditions.
<7> At least one lectin selected from the group consisting of (a) to (c) below:
(a) a lectin comprising the amino acid sequence as set forth in SEQ ID NO:14;
(b) a lectin comprising an amino acid sequence having a homology of 90% or more to the amino acid sequence as set forth in SEQ ID NO:14; and
(c) a lectin encoded by a DNA that hybridizes with a DNA comprising the base sequence as set forth in SEQ ID NO:37 under stringent conditions.
<8> At least one lectin selected from the group consisting of (a) to (c) below:
(a) a lectin comprising the amino acid sequence as set forth in SEQ ID NO:38;
(b) a lectin comprising an amino acid sequence having a homology of 90% or more to the amino acid sequence as set forth in SEQ ID NO:38; and
(c) a lectin encoded by a DNA that hybridizes with a DNA comprising the base sequence as set forth in SEQ ID NO:39 under stringent conditions.
<9> At least one lectin selected from the group consisting of (a) to (c) below:
(a) a lectin comprising the amino acid sequence as set forth in SEQ ID NO:40;
(b) a lectin comprising an amino acid sequence having a homology of 90% or more to the amino acid sequence as set forth in SEQ ID NO:40; and
(c) a lectin encoded by a DNA that hybridizes with a DNA comprising the base sequence as set forth in SEQ ID NO:41 under stringent conditions.
<10> At least one lectin selected from the group consisting of (a) to (c) below:
(a) a lectin comprising the amino acid sequence as set forth in SEQ ID NO:42;
(b) a lectin comprising an amino acid sequence having a homology of 90% or more to the amino acid sequence as set forth in SEQ ID NO:42; and
(c) a lectin encoded by a DNA that hybridizes with a DNA comprising the base sequence as set forth in SEQ ID NO:43 under stringent conditions.
<11> At least one lectin selected from the group consisting of (a) to (c) below:
(a) a lectin comprising the amino acid sequence as set forth in SEQ ID NO:44;
(b) a lectin comprising an amino acid sequence having a homology of 90% or more to the amino acid sequence as set forth in SEQ ID NO:44; and
(c) a lectin encoded by a DNA that hybridizes with a DNA comprising the base sequence as set forth in SEQ ID NO:45 under stringent conditions.
<12> At least one lectin selected from the group consisting of (a) to (c) below:
(a) a lectin comprising the amino acid sequence as set forth in SEQ ID NO:46;
(b) a lectin comprising an amino acid sequence having a homology of 90% or more to the amino acid sequence as set forth in SEQ ID NO:46; and
(c) a lectin encoded by a DNA that hybridizes with a DNA comprising the base sequence as set forth in SEQ ID NO:47 under stringent conditions.
<13> At least one lectin selected from the group consisting of (a) to (c) below:
(a) a lectin comprising the amino acid sequence as set forth in SEQ ID NO:48;
(b) a lectin comprising an amino acid sequence having a homology of 90% or more to the amino acid sequence as set forth in SEQ ID NO:48; and
(c) a lectin encoded by a DNA that hybridizes with a DNA comprising the base sequence as set forth in SEQ ID NO:49 under stringent conditions.
<14> At least one lectin selected from the group consisting of (a) to (c) below:
(a) a lectin comprising the amino acid sequence as set forth in SEQ ID NO:50;
(b) a lectin comprising an amino acid sequence having a homology of 90% or more to the amino acid sequence as set forth in SEQ ID NO:50; and
(c) a lectin encoded by a DNA that hybridizes with a DNA comprising the base sequence as set forth in SEQ ID NO:51 under stringent conditions.
<15> A lectin derived from a green alga (Avrainvillea capituliformis) being present in a fraction obtained by extracting the green algae with a buffer, salting out a obtained soluble fraction, dialyzing a obtained precipitate, and purifying the precipitate through gel filtration, the lectin having a molecular weight of from 15,000 to 20,000 Da as shown in reductive SDS-PAGE and displaying an agglutination activity against trypsin-treated rabbit red blood cells.
<16> A lectin derived from a green alga (Codium subtubulosum) being present in a fraction obtained by extracting the green algae with a buffer, salting out a obtained soluble fraction, dialyzing a obtained precipitate, followed by adsorption of the precipitate on a column immobilized with submaxillary mucin and then elution with N-acetyl-D-galactosamine, the lectin having a molecular weight of from 10,000 to 15,000 Da and displaying an agglutination activity against trypsin-treated rabbit red blood cells.
<17> A lectin comprising the lectin according to any one of <4> to <16> and an additional functional protein fused thereto. <18> A DNA encoding the lectin according to any one of <4> to <17>.

### Advantageous Effects of Invention

The present invention makes possible a method capable of distinguishing between species within the genus Staphylococcus, particularly a method capable of distinguishing species within the genus Staphylococcus rapidly and conveniently.

### BRIEF DESCRIPTION OF DRAWINGS

FIG. 1 shows a graph illustrative of binding affinities of Eschericha coli and Pseudomonas aeruginosa to various types of lectins.

FIG. 2 shows a graph illustrative of binding affinities of Bacillus subtilis and Staphylococcus aureus (ATCC6538 strain) to various types of lectins.

FIG. 3 shows a graph illustrative of binding affinities of Staphylococcus aureus (ATCC27217 strain) and Staphylococcus epidermidis (ATCC12228 strain) to various types of lectins.

FIG. 4 shows a graph illustrative of binding affinities of Staphylococcus epidermidis (ATCC14990 strain) and Staphylococcus capitis (ATCC27840 strain) to various types of lectins.

FIG. 5 shows a graph illustrative of binding affinities of Staphylococcus capitis (ATCC35661 strain) to various types of lectins.

FIG. 6 shows radar charts illustrative of binding affinities of Staphylococci and others to various types of lectins.

FIG. 7 shows a plot diagram illustrative of data of binding affinities between species of the genus Staphylococcus and Tachylectin-2 as analyzed by the Tukey-Kramer multiple comparison method.

FIG. 8 shows a graph illustrative of the results obtained by examining the discriminability of Staphylococcus aureus (ATCC6538 strain) in a plate well (Blank) where no lectins are immobilized, in the presence of Staphylococcus epidermidis (ATCC12228 strain). Note that because neither Staphylococcus epidermidis nor Staphylococcus aureus binds to the plate (Blank) where no lectins are immobilized, it has been used as negative controls in the tests shown in FIGS. 9 to 11.

FIG. 9 shows a graph illustrative of the results obtained by examining the discriminability of Staphylococcus aureus (ATCC6538 strain) in a plate well immobilized with the anti-S. epidermidis serum, in the presence of Staphylococcus epidermidis (ATCC12228 strain).

FIG. 10 shows a graph illustrative of the results obtained by examining the discriminability of Staphylococcus aureus (ATCC6538 strain) in a plate well immobilized with PNA, in the presence of Staphylococcus epidermidis (ATCC12228 strain).

FIG. 11 shows a graph illustrative of the results obtained by examining the discriminability of Staphylococcus aureus (ATCC6538 strain) in a plate well immobilized with algMPL, in the presence of Staphylococcus epidermidis (ATCC12228 strain).

FIG. 12 shows a graph illustrative of the results obtained by examining the discriminability of Staphylococcus aureus (ATCC6538 strain) in a plate well (Blank) where no lectins are immobilized, in the presence of Staphylococcus epidermidis (ATCC12228 strain) in milk. Note that the test results shown in FIG. 12 are negative controls in tests shown in FIGS. 13 to 15.

FIG. 13 shows a graph illustrative of the results obtained by examining the discriminability of Staphylococcus aureus (ATCC6538 strain) in a plate well immobilized with the anti-S. epidermidis serum, in the presence of Staphylococcus epidermidis (ATCC12228 strain) in milk.

FIG. 14 shows a graph illustrative of the results obtained by examining the discriminability of Staphylococcus aureus (ATCC6538 strain) in a plate well immobilized with PNA, in the presence of Staphylococcus epidermidis (ATCC12228 strain) in milk.

FIG. 15 shows a graph illustrative of the results obtained by examining the discriminability of Staphylococcus aureus (ATCC6538 strain) in a plate well immobilized with algMPL, in the presence of Staphylococcus epidermidis (ATCC12228 strain) in milk.

### DESCRIPTION OF EMBODIMENTS

<Method for Distinguishing Between Species within the Genus Staphylococcus>

The method for distinguishing between species within the genus Staphylococcus according to the present invention is a method for distinguishing species within the genus Staphylococcus based on binding affinity to at least one lectin as an indicator, the lectin being selected from the group consisting of Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, algMPL, and algCSA.

In the present invention, “distinguishing between species” means that a specific species or plural species are targeted and lectin(s) according to the present invention is used singly or are used in combinations of plural numbers to determine the presence or absence of the one or plural species.

The “species within the genus Staphylococcus” according to the present invention are species belonging to the genus Staphylococcus. Examples include Staphylococcus aureus, Staphylococcus epidermidis, Staphylococcus capitis, Staphylococcus lugdunensis, Staphylococcus caprae, Staphylococcus warneri, Staphylococcus hominis, and Staphylococcus haemolyticus. In the present invention, among these it is preferable to identify at least one species selected from the group consisting of Staphylococcus aureus, Staphylococcus epidermidis, Staphylococcus capitis, and Staphylococcus hominis.

The specimen with which the species within the genus Staphylococcus can be distinguished by the method of the present invention is not particularly limited insofar as it contains or is suspected to contain “the species within the genus Staphylococcus”; and it can be appropriately selected or prepared, depending upon the objective. For example, in cases where the food hygiene inspection is intended, there may be mentioned the food, an extract from the food, a culture from the food, a wipe sample of appliances with which the food is treated, and a culture from the sample. In cases where the examination of patients having infection is intended, there may be mentioned biological samples collected from the patient (a blood sample, a saliva sample, a urine sample, a feces sample, a mucosa-related lymphoid tissue sample, a cerebrospinal fluid sample, a synovial fluid sample, a pleural fluid sample, and a secretory fluid sample from suppuration wound), and cultures from these samples. Note that when the culture from the aforementioned sample is prepared, the “medium for culturing the specimen” to be described later can be appropriately selected and utilized.

Further, as will be shown in the Examples to be described later, the “species within the genus Staphylococcus” may either be at the state of stationary phase or at the state of logarithmic growth phase in the specimen with which the species within the genus Staphylococcus can be distinguished by the method of the present invention.

The stationary phase is a stage at which the number of live cells does not increase, a stage at which the number of divided nascent cells is equal to the number of extinct cells, or a stage at which the division of cells has ceased. Growth of bacteria in nature generally forms a colony by attaching to the surface layer of some sort; therefore, a visible colony is at the state of stationary phase. Moreover, at the incident of food poisoning bacteria present in a food are nearly at the state of stationary phase in most instances. Accordingly, the method of the present invention can preferably be used even against foods or the like contaminated at such a level that can cause the visible colony or the food poisoning.

In contrast, the logarithmic growth phase is a stage in which binary division is repeated at a constant rate, and since the bacterial population which is at this stage is relatively homogenous, it is at the state that is suited to the analysis of bacterial properties. Accordingly, the method of the present invention can preferably be used even against a specimen that requires culturing because bacteria or cells that are at the state being suited to the analysis of their properties are small in number.

The “lectin” according to the present invention is a protein that recognizes a sugar chain and that is other than an immunoglobulin. In the present invention, it displays differing binding affinities to between species within the genus Staphylococcus. There is used at least one protein selected from the group consisting of Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, algMPL, and algCSA.

The “Tachylectin-2” according to the present invention is a lectin comprising the amino acid sequence as set forth in SEQ ID NO:1 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:1.

The homology of sequence can be determined by utilizing a BLASTP (amino acid level) program (Altschul et al., J. Mol. Biol., 215:403-410, 1990). The program is based on an algorism BLAST by Karlin and Altschul (Proc. Natl. Acad. Sci. USA, 87: 2264-2268, 1990; Proc. Natl. Acad. Sci. USA, 90: 5873-5877, 1993) When amino acid sequences are analyzed by BLAST, the parameter is set to, for example, score=50 and wordlength=3. Further, when amino acid sequences are analyzed by using the Gapped BLAST program, it can be carried out as is described by Altschu et al. (Nucleic Acids Res. 25: 3389-3402, 1997). When BLAST and the Gapped BLAST program are used, default parameters of the respective programs are used. Concrete techniques of these analytical methods are known (and so forth). One skilled in the art can also obtain the lectin comprising an amino acid sequence having a homology of 90% or more to the amino acid sequence as set forth in SEQ ID NO:1 as well as the lectin comprising the amino acid sequence as set forth in SEQ ID NO:1 on the basis of base sequences of DNAs encoding these lectins in accordance with a preparation method for the lectins which will be described in <Lectins and DNAs Encoding the Lectins> (and so forth).

The “LEL” according to the present invention is a lectin comprising the amino acid sequence as set forth in SEQ ID NO:2 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:2.

Typical examples of the “LEL” according to the present invention include Lycopersicon esculentum (tomato) lectin (LEL. TL) (manufactured by Vector Laboratories Inc.; Catalog No. L-1170).

The “KAA1” according to the present invention is at least one lectin selected from the group consisting of (a) to (c) below:

(a) a lectin comprising the amino acid sequence as set forth in SEQ ID NO:3;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:3; and
(c) a lectin encoded by a DNA that hybridizes with a DNA comprising the base sequence as set forth in SEQ ID NO:34 under stringent conditions.

The “stringent conditions” according to the present invention are conditions under which between nucleic acids complementary bonds are formed but non-complementary bonds are not formed. Embodiments of “hybridization under stringent conditions” according to the present invention include, for example, conditions where hybridization is carried out with “6×SSC, 40% formamide, 25° C.” and washing is carried out with “1×SSC, 55° C.” More preferable conditions employ those where hybridization is carried out with “6×SSC, 40% formamide, 37° C.” and washing is carried out with “0.2×SSC, 55° C.”; particularly preferable conditions can employ those where hybridization is carried out with “6×SSC, 50% formamide, 37° C.” and washing is carried out with “0.1×SSC, 62° C.” Note that one skilled in the art can realize stringent conditions of hybridization that are similar to the aforementioned conditions by appropriately choosing various conditions, which include salt concentrations (such as the dilution rate of SSC), the concentration of formamide and temperature. Different embodiments of “hybridization under stringent conditions” according to the present invention also include, for example, conditions where nucleic acids having extremely high homologies (e.g., between nucleic acids with homologies of 95% or more) hybridizes with each other but nucleic acids having homologies lower than the above do not hybridizes with each other (and so forth). One skilled in the art can also obtain the lectin encoded by a DNA that hybridizes with a DNA comprising the base sequence as set forth in SEQ ID NO:34 under stringent conditions in accordance with a preparation method for the lectins which will be described in <Lectins and DNAs Encoding the Lectins> (and so forth).

The “BCL11” according to the present invention is at least one lectin selected from the group consisting of (a) to (c) below:

(a) a lectin comprising the amino acid sequence as set forth in SEQ ID NO:4;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:4; and
(c) a lectin encoded by a DNA that hybridizes with a DNA comprising the base sequence as set forth in SEQ ID NO:35 under stringent conditions.

The “CBA” according to the present invention is at least one lectin selected from the group consisting of (a) to (c) below:

(a) a lectin comprising the amino acid sequence as set forth in SEQ ID NO:38;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:38; and
(c) a lectin encoded by a DNA that hybridizes with a DNA comprising the base sequence as set forth in SEQ ID NO:39 under stringent conditions.

The lectin encoded by the DNA comprising the base sequence as set forth in SEQ ID NO:39 is a precursor, and its mature type lectin is a lectin comprising the amino acid sequence as set forth in SEQ ID NO:38 or the like.

The “CBA” according to the present invention is a lectin that is obtained by the separation, extraction, and purification from Codium barbatum and that is detected as a single band, respectively, between molecular weights of 6.5 kDa and 14.3 kDa in reductive SDS-PAGE and between 14.3 kDa and 20.1 kDa in non-reductive SDS-PAGE. Further, it is a lectin that has an activity of agglutinating trypsin-treated red blood cells and that has its hemagglutination activity inhibited by porcine asialo-thyroglobulin, namely displaying specificity for the porcine asialo-thyroglobulin. The “CBA” according to the present invention has, for example, a lowest concentration of 781 ng/ml that is capable of agglutinating red blood cells and that has its hemagglutination inhibited by the porcine asialo-thyroglobulin at 30 μg/ml.

As the method of preparing “CBA” according to the present invention, there is mentioned a method described below, for example. Specifically, one kilogram by wet weight of Codium barbatum is first frozen in liquid nitrogen, powdered, and stirred overnight by adding 500 ml of a buffer of 20 mM Tris-HCl and 150 mM NaCl (TBS, pH 7.5). Next, the obtained mixture is centrifuged at 13,500 g for 30 min and a supernatant is recovered. After the addition of ammonium sulfate to bring it to saturation at a final concentration of 75% and stirring for 30 min, it is allowed to stand overnight and is then centrifuged at 13,500 g for 30 min to recover precipitates. The recovered precipitates are further dissolved in a small amount of TBS and are dialyzed with TBS to remove ammonium sulfate. Next, after the obtained dialysate is centrifuged at 10,000 g for 30 min to remove the precipitates, it is dialyzed against a buffer of 20 mM Tris-HCl and 1 M (NH₄)₂SO₄ (pH 7.5), passed through a 3.31 ml of TSKgel Phenyl-5PW column (7.5×75 mm) and is eluted with a gradient of 1-0 M (NH₄)₂SO₄ at a flow rate of 0.5 ml/min. Fractions having hemagglutination activity are further collected and dialyzed against 20 mM Tris-HCl and 0.85% NaCl buffer (pH 7.5), whereby the “CBA” according to the present invention can be purified from Codium barbatum.

The “HAA” according to the present invention is a lectin that is obtained by the separation, extraction, and purification from petit-gris and that is detected as a single band in immunoelectrophoresis against an anti-albumin gland. It is also a lectin that has an activity of agglutinating A1 and A2 cells and further that has its agglutination activity inhibited by N-acetyl-D-galactosamine, namely displaying specificity for N-acetyl-D-galactosamine.

The “HAA” according to the present invention has, for example, a lowest concentration of 0.5 μg/ml that is capable of agglutinating the A1 and A2 cells and that has its A1 and A2 cell agglutination activity inhibited by 20 mM N-acetyl-D-galactosamine. Typical examples of the “HAA” according to the present invention include a lectin derived from escargot (Helix aspersa) (manufactured by Sigma-Aldrich Corporation; product No. L6635).

As the method of preparing “HAA” according to the present invention, there is mentioned a method described below, for example. Specifically, 20 ml of an albumin gland extract of petit-gris exhibiting an agglutination activity at 1/4,000 concentration thereof is first passed through 600 ml of Sephadex G-200 (3.8 cm×53 cm) equilibrated with 0.01 M Tris buffer (pH 8.0) at a flow rate of 15 ml/h and is eluted with the Tris buffer. Note that the eluate from the Tris buffer has no hemagglutination activity and that fractions having the agglutination activity are obtained after elution with 0.002 M N-acetyl-D-glucosamine at the same flow rate followed by carrying out re-elution with a 500 ml-flow. After the thus-collected active fractions are dialyzed with distilled water, they are dried at 40° C. using a rotary evaporator, whereby the “HAA” according to the present invention can be purified as a solid from petit-gris.

The “HPA” according to the present invention is a lectin comprising the amino acid sequence as set forth in SEQ ID NO:5 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:5.

Typical examples of the “HPA” according to the present invention include Pure Helix pomatia lectin (snail)—HPA—(manufactured by EY Laboratories, Inc.; Catalog No. L-3601).

The “STL” according to the present invention is a lectin comprising the amino acid sequence as set forth in SEQ ID NO:6 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:6.

Typical examples of the “STL” according to the present invention include Solanum tuberosum (potato) lectin (manufactured by Vector Laboratories Inc.; Catalog No. L-1160).

The “proBCA1” according to the present invention is a lectin comprising the amino acid sequence as set forth in SEQ ID NO:7 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:7.

The “proBCA1” according to the present invention is a precursor and its mature type lectin (BCA1) is a lectin comprising an amino acid sequence of the 1st to the 125th positions as set forth in SEQ ID NO:7 or an amino acid sequence having a homology of 90% or more to the amino acid sequence of the 1st to the 125th positions as set forth in SEQ ID NO:7.

The “proBCA2” according to the present invention is a lectin comprising the amino acid sequence as set forth in SEQ ID NO:8 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:8. Note that the “proBCA2” according to the present invention is a precursor and its mature type lectin (BCA2) is a lectin comprising the amino acid sequence as set forth in SEQ ID NO:9 or an amino acid sequence having a homology of 90% or more to the amino acid sequence as set forth in SEQ ID NO:9.

The “ULL” according to the present invention is a lectin comprising the amino acid sequence as set forth in SEQ ID NO:10 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:10.

The “DSA” according to the present invention is a lectin that is obtained by the separation, extraction, and purification from Datura stramonium and that displays two bands at molecular weights of 46 kDa and 40 kDa in reductive SDS-PAGE and a molecular weight of 86 kDa in non-reductive SDS-PAGE. Specifically, the “DSA” according to the present invention is a dimer through a disulfide bond. Further, the “DSA” according to the present invention is a lectin that has an activity of agglutinating human type O red blood cells and that binds specifically to β(1,4)-bonded N-acetyl-D-glucosamine. The “DSA” according to the present invention has, for example, a lowest concentration of 30 μg/ml that is capable of agglutinating human O type red blood cells. Typical examples of the “DSA” according to the present invention include DSA (manufactured by Seikagaku Corporation; Catalog No. 300037).

As the method of preparing “DSA” according to the present invention, there is mentioned a method described below, for example. Specifically, 200 g of the seeds of Datura stramonium is extracted with 500 ml of methanol four times and the residual seeds are washed with 250 ml of dichloromethane and dried. Fifteen grams of polyvinylpolypyrolidone is added to the dried seeds and mixed, and are extracted with 700 ml of PBS overnight. The extract is centrifuged at 11,000 g for 20 min and the remaining precipitates are extracted with 500 ml of PBS again. The obtained extract is mixed and dialyzed with 0.01 M acetic acid. Brown precipitates resulting from the dialysis are separated by centrifugation and a centrifuged supernatant is dialyzed with PBS again. The extract containing the lectin is passed through an N,N′-diacetyl-chitobioside-Sepharose column at a flow rate of 20 ml/h, is washed with PBS and is gradually eluted with N-acetyl-D-glucosamine oligomer. Note that in this case, because the “DSA” according to the present invention is contained in fractions eluted by 1 mg/ml of the oligomer after washing the column with PBS, the fractions are collected and are dialyzed with PBS. Further, 10-12 ml of the dialyzed lectin solution is gel-filtered and purified by Sephadex G-200 super fine column (2.5 cm×86 cm), whereby the “CBA” according to the present invention can be purified from Datura stramonium.

The “PWM” according to the present invention is a lectin that is obtained by the separation, extraction, and purification from pokeweed (Phytolacca americana) and that is detected as five bands at molecular weights of 22,000±3300, 31,000±4600, 25,000±3700, 21,000±3200, and 19,000±2900 in SDS-PAGE. Also, the “PWM” according to the present invention is a lectin that has an activity of agglutinating blood cells with blood type non-specificity (ABO types) and that has its hemagglutination activity inhibited by 1-4 bonded-N-acetyl-D-glucosamine or N-acetyllactosamine, namely displaying specificity for N-acetyl-D-glucosamine or N-acetyllactosamine. The “PWM” according to the present invention is also a lectin having mitogenic activity. As the “PWM” according to the present invention, there is mentioned, for example, those of which the minimum values of hemagglutination activities and mitogenic activities are shown in Table 1 below. Typical examples of the “PWM” according to the present invention include PWM (manufactured by Seikagaku Corporation; Catalog No. 300141).

TABLE 1

| | | Molecular | Hemagglutination | Mitogenic |
|---|---|---|---|---|
| | | Weight | Activity | Activity |
| Fraction | Yield | (Da) | (ng/ml) | (μg/ml) |
| pa-1 | 184 mg | 22,000 ± 3300 | 150 | 10-100 |
| pa-2 | 290 mg | 31,000 ± 4600 | 310 | 1-100 |
| pa-3 | 19 mg | 25,000 ± 3700 | 1,250 | 10-100 |
| pa-4 | 277 mg | 21,000 ± 3200 | >166,000 | 50-1,000 |
| pa-5 | 48 mg | 19,000 ± 2900 | 2,500 | 50-500 |

As the method of preparing “PWM” according to the present invention, there is mentioned a method described below, for example. Specifically, the roots of pokeweed harvested in early autumn through winter are first ground, extracted after addition of PBS and further dialyzed with water; and a supernatant is recovered with brown precipitates being left. The obtained supernatant is passed through a 5×30 cm hydroxyapatite column (Bio-Gel HT; manufactured by Bio-Rad Laboratories), followed by elution with 5 mM potassium phosphate (pH 7.8) and further

with 50 mM potassium phosphate (pH 7.8). Note that hemagglutination activity and mitogenic activity are noted in the obtained fraction. Next, this fraction is dialyzed with water and dried to produce a solid; and the obtained solid is dissolved in 2-5 ml of PBS and it is passed through 2.5×90 cm Sephadex G-75 to carry out gel filtration, whereby the “PWM” according to the present invention can be purified from pokeweed.

The yield examples of the five fractions obtained by such purification (pa-1, pa-2, pa-3, pa-4, and pa-5: Each number

indicating the order of elution in the Sephadex G-75 gel filtration.) from 1 kg of pokeweed roots are shown in Table 1.

The “UDA” according to the present invention is a lectin comprising the amino acid sequence as set forth in SEQ ID NO:11 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:11. Typical examples of the “UDA” according to the present invention include Pure Urtica dioica lectin (Stinging Nettle)—UDA—(manufactured by EY Laboratories, Inc.; Catalog No. L-8005).

The “WFL” according to the present invention is a lectin that is obtained by the separation, extraction, and purification from Japanese wisteria and is detected as a single band at pH 9.4, 8.0, or 4.0 in polyacrylamide electrophoresis as well as that displays a molecular weight of 32 kDa in reductive SDS-PAGE and displays a molecular weight of 68 kDa in non-reductive SDS PAGE. Also, the “WFL” according to the present invention is a lectin that has an activity of agglutinating human A1 red blood cells and that has its agglutination activity inhibited by N-acetyl-D-galactosamine, namely displaying specificity for N-acetyl-D-galactosamine. The “WFL” according to the present invention, for example, has a lowest concentration of 15-30 μg/ml that is capable of agglutinating human A1 red blood cells and has its agglutination activity inhibited by 63 μg/ml of N-acetyl-D-galactosamine. Typical examples of the “WFL” according to the present invention include Pure Wisteria floribunda lectin (Japanese wisteria)—WFA—(manufactured by EY Laboratories, Inc.; Catalog No. L-3101).

As the method of preparing “WFL” according to the present invention, there is mentioned a method described below, for example. Specifically, Japanese wisteria seeds are pulverized and mixed into 0.1 M Tris buffer (pH 7.5). After allowing it to stand overnight, a centrifuged supernatant is salted out with 40% ammonium sulfate and the obtained supernatant is further salted out with 70% ammonium sulfate. Note that in this instance, 70% of hemagglutination activity remains in the obtained precipitate. Further, 80% saturated ammonium sulfate is added to the obtained fraction, and it is passed through a Celite 545 column, followed by elution with lowering ammonium sulfate concentrations. Next, fractions with from 60% to 50% ammonium sulfate concentrations are collected, and they are dialyzed with 0.1 M Tris-buffer (pH 7.5) and concentration is conducted by ultrafiltration. The Celite eluate is further passed through DEAE Sepharose A-50 and elution is conducted with a gradient of from 0 M to 0.6 M NaCl. Further, fractions of 0.25 M elution, which have hemagglutination activity, are collected and subjected to gel filtration purification by being passed through Sephadex G-200 column, whereby the “WFL” according to the present invention having a main peak with recognizable hemagglutination activity can be purified from Japanese wisteria.

The “hypninA3” according to the present invention is a lectin comprising the amino acid sequence as set forth in SEQ ID NO:12 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:12.

The “Tachylectin-3” according to the present invention is a lectin comprising the amino acid sequence as set forth in SEQ ID NO:52 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:52.

The “OAA” according to the present invention is a lectin comprising the amino acid sequence as set forth in SEQ ID NO:53 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:53.

The “PNA” according to the present invention is a lectin comprising the amino acid sequence as set forth in SEQ ID NO:54 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:54. Typical examples of the “PNA” according to the present invention include PNA, Arachis hypogaea Agglutinin (manufactured by Seikagaku Corporation; Code No. J114).

The “TL” according to the present invention is a lectin that is obtained by the separation, extraction, and purification from tulip and that displays a molecular weight of 28,000 in reductive SDS-PAGE as well as displays a molecular weight of around 30,000 by molecular weight determination through gel filtration, to which 6 M guanidinium chloride has been added. It is also a lectin that is observed to be most strongly inhibited by N-acetyl-D-galactosamine in a hemagglutination inhibition test using human type O red blood cells (e.g., inhibition at a concentration of 0.2 mM). Typical examples of the “TL” according to the present invention include Pure Tulipa sp. Lectin (Tulip)—TL—(manufactured by EY Laboratories, Inc.; Catalog No. L-8001).

As the method of preparing “TL” according to the present invention, there is mentioned a method described below, for example. Specifically, 50 g of tulip bulbs are first fragmented finely and homogenized in 250 ml of PBS containing 5 mM thiourea (1.5 mM KH₂PO₄, 10 mM Na₂PO₄, 3 mM KCl, 140 mM NaCl, pH 7.4). After allowing it to stand on ice for 30 min, a supernatant is transferred to a different container, and an equal amount of PBS is added to precipitates for extraction again. Two extracts are mixed and are centrifuged at 20,000 g at a low temperature for one hour. A supernatant is recovered and frozen at −80° C. overnight. After thawing, a sample is centrifuged at 100,000 g for 30 min, and a supernatant is passed through a 10 ml fetuin-agarose column (7.5×75 mm) equilibrated with PBS. The column is washed with PBS until absorbance at 280 reaches 0.01 or less and is eluted with 20 mM 1,3-diaminopropane (DAP). The pH of the eluted fractions is adjusted to 8.7 with 0.1 M HCl and they are passed through DEAE-Bio-gel equilibrated with 10 mM 2-amino-2-(hydroxymethyl)-1,3-propanediol (Tris)-HCl (pH 8.7), followed by extraction with a concentration gradient of 0-0.5 MNaCl, whereby the “TL” according to the present invention can be purified from tulip. For example, 2 mg of TL can be obtained from 1 g of tulip bulbs by such purification method.

The “ACG” according to the present invention is a lectin comprising the amino acid sequence as set forth in SEQ ID NO:55 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:55.

The “AC-avranin” according to the present invention is a lectin derived from a green alga (Avrainvillea captituliformis). The lectin is present in fractions obtained by extracting the green algae with buffer, salting out the obtained soluble fractions, dialyzing and purifying with gel filtration, the obtained precipitate; it has a molecular weight of from 15,000 to 20,000 Da as shown in reductive SDS-PAGE and displays an agglutination activity against trypsin-treated rabbit red blood cells.

Examples of the “AC-avranin” according to the present invention include a lectin obtained by a purification method described below. Specifically, a seaweed (Avrainvillea captituliformis) is first frozen-pulverized, to which a buffer (e.g., Tris buffer or a phosphate buffer, with pH 7-8). After stirring (e.g., after stirring overnight at 4° C.), centrifugation is carried out to obtain a supernatant as extract. Next, to the obtained extract is added an inorganic salt (e.g., ammonium sulfate, ammonium chloride, sodium sulfate sodium chloride, or potassium chloride) at stirring so as to provide a predetermined saturated concentration (e.g., 70-80%). After stirring, it is allowed to stand (e.g., standing overnight) and thereby to be salted out. Further, this is centrifuged, and the obtained precipitates are dissolved in a small amount of a buffer (e.g., a Tris buffer or a phosphate buffer, with pH 7-8) and are sufficiently dialyzed against the buffer. Next, an internal solution is recovered as a salted-out fraction and the salted-out fraction is subjected to gel filtration. Moreover, the absorbance at a wavelength of 280 nm and the agglutination activity against trypsin-treated rabbit red blood cells are used as indicators to select a lectin, which is present in the eluted fraction by gel filtration: it is a typical example of the “AC-avranin” according to the present invention to be mentioned.

The “MOA” according to the present invention is a lectin comprising the amino acid sequence as set forth in SEQ ID NO:56 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:56. Typical examples of the “MOA” according to the present invention include Pure Marasmium oreades agglutinin Lectin (mushroom)—MOA—(manufactured by EY Laboratories, Inc.; Catalog No. L-9001).

The “API 144” according to the present invention is a lectin comprising the amino acid sequence as set forth in SEQ ID NO:57 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:57.

The “CV-N” according to the present invention is a lectin comprising the amino acid sequence as set forth in SEQ ID NO:58 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:58.

The “PMA” according to the present invention is a lectin comprising the amino acid sequence as set forth in SEQ ID NO:59 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:59. Typical examples of the “PMA” according to the present invention include Pure Polygonatum mulitiflorum Lectin (Common Solomon's Seal)—PMA—(manufactured by EY Laboratories, Inc.; Catalog No. L-8009).

The “Garlic lectin” according to the present invention is a lectin comprising the amino acid sequence as set forth in SEQ ID NO:60 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:60.

The “GSL-II” according to the present invention is a lectin comprising the amino acid sequence as set forth in SEQ ID NO:15 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:15. Typical examples of the “GSL-II” according to the present invention include Unconjugated Griffonia (Bandeiraea) simplicifolia Lectin (GSL) II (manufactured by Vector Laboratories Inc.; Catalog No. L-1210) and Pure Griffonia simplicifolia Lectin—GS-II—(manufactured by EY Laboratories, Inc.; Catalog No. L-2402).

The “PAA” according to the present invention is a lectin that is obtained by the separation, extraction and purification from avocado and that displays the amino acid composition shown in Table 2. Also, as Table 3 shows, it is a lectin that displays hemagglutination activity against various types of red blood cells. Typical examples of the “PAA” according to the present invention include Crude Perseau americana Lectin (Avocado)—PAA—(manufactured by EY Laboratories, Inc.; Catalog No. L-6100)

TABLE 2
Amino Acid Composition Analytical Data for PAA

| | Proportion |
|---|---|
| Amino Acid | (nmol/mg) |
| alanin | 13.2 |
| arginine | 4.0 |
| aspartic acid | 22.5 |
| cysteine | 3.2 |
| glutamic acid | 60.6 |
| glycine | 54.7 |
| histidine | 2.7 |
| isoleucine | 4.4 |
| leucine | 7.1 |
| lysine | 5.9 |
| methionine | 7.3 |
| phenylalanin | 2.9 |
| proline | 7.2 |
| serine | 16.2 |
| threonine | 9.4 |
| tryptophan | 0.3 |
| tyrosine | 1.4 |
| valine | 5.8 |

TABLE 3

| Red Blood Cell | Dilution Ratio |
|---|---|
| human type A | 128 |
| human type B | 32 |
| human type O | 32 |
| mouse | 128 |
| goat | 128 |
| cattle | 128 |
| rat | 8 |
| rabbit | 64 |

As the method of preparing “PAA” according to the present invention, there is mentioned a method described below, for example. Specifically, the testas of avocado seeds are first removed, lyophilized and finely powdered. The powdered seeds (100 g) are suspended in 1.0 L of water or PBS, stirring is carried out at 4° C. for 16-20 h, and solids are removed by centrifugation. After 800 mL of a supernatant is dialyzed with 5 L of water five times, lyophilization is carried out to obtain a solid having lectin activity (e.g., 800-1200 mg), whereby the “PAA” according to the present invention can be purified from avocado. The thus-obtained extract by purification is dissolved in PBS so as to give a concentration of 1.0 mg/ml. The obtained solution (50 μl) is used to test a hemagglutination activity against each 50 μl of various types of red blood cells that are diluted to 2%. As a result, the hemagglutination activities are displayed against the various types of red blood cells, as shown in Table 3, for example. Note that the dilution ratios described in Table 3 represent numbers of serial dilution of the above-mentioned extract with PBS.

The “UEA-II” according to the present invention is a lectin comprising the amino acid sequence as set forth in SEQ ID NO:61 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:61. Typical examples of the “UEA-II” according to the present invention include Pure Ulex europaeus Lectin (Gorse, Furze)—UEA-I—(manufactured by EY Laboratories, Inc.; Catalog No. L-2201).

The “RSL” according to the present invention is a lectin comprising the amino acid sequence as set forth in SEQ ID NO:62 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:62.

The “CPA” according to the present invention is a lectin comprising the amino acid sequence as set forth in SEQ ID NO:63 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:63. Typical examples of the “CPA” according to the present invention include Pure Cicer arietinum Lectin (Chick Pea)—CPA—(manufactured by EY Laboratories, Inc.; Catalog No. L-6601).

The “CHA-1” according to the present invention is a lectin comprising the amino acid sequence as set forth in SEQ ID NO:64 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:64.

The “LAA” according to the present invention is a lectin comprising the amino acid sequence as set forth in SEQ ID NO: 65 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:65. Typical examples of the “LAA” according to the present invention include Pure Laburnum alpinum Lectin (Scotch Laburnum)—LAA—(manufactured by EY Laboratories, Inc.; Catalog No. L-5301).

The “SHA” according to the present invention is a lectin that is obtained by the separation, extraction, and purification from painted sage (Salvia viridis) and that displays a molecular weight of 50,000 in non-reductive SDS-PAGE as well as displays a molecular weight of 35,000 under reductive conditions. It is also a lectin that displays an agglutination activity which is specific to A1 red blood cells. For example, while the lectin agglutinates the A1 red blood cells at a concentration of 27 μg/ml in a human red blood cell agglutination test, type 0 and type B red blood cell agglutinations are not observed but the inhibition of hemagglutination with 0.1 mM N-acetyl-D-galactosamine is observed in an inhibition test with a monosaccharide: it is a lectin to be mentioned as the “SHA” according to the present invention. Typical examples of the “SHA” according to the present invention include Pure Salvia horminum—SHA—(manufactured by EY Laboratories, Inc.; Catalog No. L-3401).

As the method of preparing “PAA” according to the present invention, there is mentioned a method described below, for example. Specifically, 30 g of painted sage seeds are first ground with a blender, to which 400 ml of PBS containing 5 mM EDTA is added, followed by stirring overnight to be conducted. The stirred solution is centrifuged at 20,000 g for 30 min, and 400 ml of PBS containing 5 mM EDTA is again added to the precipitates, followed by stirring overnight to be conducted. Further, after the two centrifuge supernatants are mixed and frozen overnight at −20° C., the mixture is thawed and is centrifuged at 3,500 g for 30 min to remove precipitates. The obtained supernatant is added to an equal amount of ethanol. A supernatant is recovered by centrifugation at 20,000 g for 30 min, and ethanol having a final concentration of 80% is further added to the supernatant. Standing overnight at 4° C. and centrifugation at 20,000 g for 30 min produces precipitates. The obtained precipitates are dissolved in water and dialyzed with water for 3 days, followed by lyophilization. 40 mg of the obtained lyophilized product is dissolved in 15 mM Tris-HCl buffer (pH 7.3), and centrifugation is carried out at 3,500 g for 30 min to recover a supernatant, which is filtered with a 0.2 μm nitrocellulose filter. The obtained sample is passed through a DEAE-TSK 545 column (2.15×15 cm) equilibrated with 15 mM Tris-HCl buffer (pH 7.3) at a flow rate of 2 ml/min, room temperature. The sample passed through the column is collected and concentrated using a PM-10 Diaflo membrane (manufactured by Amicon Inc.). The concentrate is passed through a GalNAc-Synsorb column (0.5×5 cm) equilibrated with TBS, and after washing with TBA, the adsorbed lectin is eluted with TBS containing 0.1 M GalNAC and is dialyzed with TBS, whereby the “SHA” according to the present invention, for example 1.5 mg, can be isolated from painted sage.

The “LPA” according to the present invention is a lectin that is obtained by the separation, extraction, and purification from Atlantic horseshoe crab and that displays a molecular weight of 33 kDa. The “LPA” according to the present invention is also a lectin that displays an agglutination activity against sheep red blood cells. Typical examples of the “LPA” according to the present invention include Pure Limulus polyphemus Lectin (Horseshoe Crab)—LPA—(manufactured by EY Laboratories, Inc.; Catalog No. L-1501).

As the method of preparing “LPA” according to the present invention, there is mentioned a method described below, for example. Specifically, blood is first collected from Atlantic horseshoe crab by cardiac puncture, and hemocyanin is separated by ultra-centrifugation at 141,000 g for 16 h or centrifugation at 30,000 g for 30 min where polyethylene glycol-8000 (PEG) is added. The separated supernatant is added to 10% PEG and centrifugation is carried out at 30,000 g for 30 min. The precipitates are dissolved in buffer A (0.15 MNaCl, 10 mM CaCl₂, 50 mM Tris, pH 8.0) and it is passed through 0.2-folds of Sepharose 4B equilibrated with buffer A. Further, the fraction having passed through is mixed with 0.1-folds of the plasma volume of phosphoryl ethanolamine-agarose to have proteins adsorbed thereon, which are washed with buffer A containing 1 M NaCl and are eluted with 0.1 M sodium citrate (pH 6.7). The obtained fraction is dialyzed with buffer A, is passed through a fetuin-agarose column equilibrated with buffer A and is eluted with 0.1 M sodium citrate (pH 6.7), whereby the “LPA” according to the present invention can be prepared from the Atlantic horseshoe crab. For example, 1.3 mg of the purified “LPA” can be also obtained from 519 mg of protein contained in the plasma by such purification method.

The “DBA” according to the present invention is a lectin comprising the amino acid sequence as set forth in SEQ ID NO:66 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:66. In the present invention, typical examples of the “DBA” include DBA (Dolichos biflorus Agglutinin) (manufactured by Seikagaku Corporation; Code No. J104).

The “TPL-1” according to the present invention is a lectin comprising the amino acid sequence as set forth in SEQ ID NO:67 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:67.

The “BML11c” according to the present invention is at least one lectin selected from the group consisting of (a) to (c) below:

(a) a lectin comprising the amino acid sequence as set forth in SEQ ID NO:13;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:13; and
(c) a lectin encoded by a DNA that hybridizes with a DNA comprising the base sequence as set forth in SEQ ID NO:36 under stringent conditions.

The “BML11c” according to the present invention is at least one lectin selected from the group consisting of (a) to (c) below:

(a) a lectin comprising the amino acid sequence as set forth in SEQ ID NO:14;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:14; and
(c) a lectin encoded by a DNA that hybridizes with a DNA comprising the base sequence as set forth in SEQ ID NO:37 under stringent conditions.

The “PVL” according to the present invention is a lectin comprising the amino acid sequence as set forth in SEQ ID NO:16 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:16. Typical examples of the “PVL” according to the present invention include weeping widow lectin (Psathyrella velutina Lectin; manufactured by Wako Pure Chemical Industries, Ltd.; distributor code 165-17591).

The “LBA” according to the present invention is a lectin comprising the amino acid sequence as set forth in SEQ ID NO:68 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:68. Typical examples of the “LBA” according to the present invention include Pure Phaseolus lunatus Lectin (Lima Bean)—LBA—(manufactured by EY Laboratories, Inc.; Catalog No. L-1701).

The “UPL-1” according to the present invention is a lectin comprising the amino acid sequence as set forth in SEQ ID NO:69 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:69.

The “BPL” according to the present invention is a lectin comprising the amino acid sequence as set forth in SEQ ID NO:70 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:70. Typical examples of the “BPL” according to the present invention includes Unconjugated Bauhinia purpurea Lectin (BPL) (manufactured by Vector Laboratories Inc.; Catalog No. L-1280).

The “CFA1” according to the present invention is at least one lectin selected from the group consisting of (a) to (c) below:

(a) a lectin comprising the amino acid sequence as set forth in SEQ ID NO:42;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:42; and
(c) a lectin encoded by a DNA that hybridizes with a DNA comprising the base sequence as set forth in SEQ ID NO:43 under stringent conditions.

The lectin encoded by the DNA comprising the base sequence as set forth in SEQ ID NO:43 is a precursor and its mature type lectin is a lectin comprising the amino acid sequence as set forth in SEQ ID NO:42 or the like.

The “CFA2” according to the present invention is at least one lectin selected from the group consisting of (a) to (c) below:

(a) a lectin comprising the amino acid sequence as set forth in SEQ ID NO:44;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:44; and
(c) a lectin encoded by a DNA that hybridizes with a DNA comprising the base sequence as set forth in SEQ ID NO:45 under stringent conditions.

The lectin encoded by the DNA comprising the base sequence as set forth in SEQ ID NO:45 is a precursor and its mature type lectin is a lectin comprising the amino acid sequence as set forth in SEQ ID NO:44 or the like.

The “BanLec” according to the present invention is a lectin comprising the amino acid sequence as set forth in SEQ ID NO:71 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:71. Typical examples of the “BanLec” according to the present invention include Pure Musa acuminata Lectin (banana)—BanLec—(manufactured by EY Laboratories, Inc.; Catalog No. L-9007).

The “BCL11d” according to the present invention is at least one lectin selected from the group consisting of (a) to (c) below:

(a) a lectin comprising the amino acid sequence as set forth in SEQ ID NO:40;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:40; and
(c) a lectin encoded by a DNA that hybridizes with a DNA comprising the base sequence as set forth in SEQ ID NO:41 under stringent conditions.

The lectin encoded by the DNA comprising the base sequence as set forth in SEQ ID NO:41 is a precursor and its mature type lectin is a lectin comprising the amino acid sequence as set forth in SEQ ID NO:40 or the like.

The “FVE” according to the present invention is a lectin comprising the amino acid sequence as set forth in SEQ ID NO:72 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:72.

The “CLA” according to the present invention is at least one lectin selected from the group consisting of (a) to (c) below:

(a) a lectin comprising the amino acid sequence as set forth in SEQ ID NO:46;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:46; and
(c) a lectin encoded by a DNA that hybridizes with a DNA comprising the base sequence as set forth in SEQ ID NO:47 under stringent conditions.

The “Pro-CFA I” and “Pro-CFA II” according to the present invention are peptidyl glycan agglutinins that are obtained by the separation, extraction, and purification from Polyopes prolifera, a red algae, and that display nearly single bands, although broad, at molecular weights of 800,000 in agarose gel electrophoresis and paper electrophoresis as well as display positive in Alcian blue staining. The “Pro-CFA I” and “Pro-CFA II” according to the present invention are also lectins displaying pronase treatment dependent agglutination activity.

As the method of preparing “Pro-CFA I” and “Pro-CFA II” according to the present invention, there is mentioned a method described below, for example. Specifically, Polyopes prolifera, which is a red alga, is first lyophilized and then is made into algal powders with a ball mill. The algal powders (100 g) are stirred overnight at 4° C. in a 20 mM phosphate buffer (PBS, pH 7.0) containing a 10-fold volume of 0.85% NaCl. This is centrifuged (6000 rpm×40 min) to obtain a supernatant as a primary extract. The extraction residue is subjected to the same extraction manipulation and the extraction is repeated 14 times until no hemagglutination activity of the extract is detected. The thus-obtained extraction residue is added to 1 L of 0.05% actinase E and stirring is carried out at 37° C. for 24 h. This is centrifuged (6,000 rpm×40 min) to obtain a supernatant, to which is added p-amidinophenylmethanesulfonyl fluoride (a protease inhibitor) so that 5 nM is attained. The pronase-treated extract is subjected to salting out with 80% saturated ammonium sulfate, and the obtained precipitates are sufficiently dialyzed against PBS to obtain an internal solution as an ammonium sulfate-salted out fraction. The ammonium sulfate-salted out fraction is added to an asialofetuin-immobilized column (1×10 cm) equilibrated with PBS, and after washing the column with PBS sufficiently, it is eluted with 1 MNaCl in PBS. The 1 MNaCl-eluted fraction (active fraction) is sufficiently dialyzed against distilled water and then is concentrated by ultrafiltration. This is added to a TOYOPEARL HW-65 column (2.2×9.2 cm) equilibrated with PBS and is eluted with PBS. Further, an active peak obtained by gel filtration is recovered and concentrated by ultrafiltration, and then it is infused to a TSKgel DEAE-5PW column (7.5×75 mm) equilibrated with a 20 mM phosphate buffer. After washing the column with the buffer sufficiently, an elution program is prepared between 0-2M NaCl in the buffer and elution is carried out using the program. Then, two saccharide peaks displaying agglutination activity are respectively recovered from the eluate, whereby the “Pro-CFA I” and “Pro-CFA II” according to the present invention can be purified from Polyopes prolifera.

The “MPA1” according to the present invention is at least one lectin selected from the group consisting of (a) to (c) below:

(a) a lectin comprising the amino acid sequence as set forth in SEQ ID NO:48;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:48; and
(c) a lectin encoded by a DNA that hybridizes with a DNA comprising the base sequence as set forth in SEQ ID NO:49 under stringent conditions.

The lectin encoded by the DNA comprising the base sequence as set forth in SEQ ID NO:49 is a precursor, and its mature type lectin is the lectin comprising the amino acid sequence as set forth in SEQ ID NO:48 or the like.

The “MPA2” according to the present invention is at least one lectin selected from the group consisting of (a) to (c) below:

(a) a lectin comprising the amino acid sequence as set forth in SEQ ID NO:50;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:50; and
(c) a lectin encoded by a DNA that hybridizes with a DNA comprising the base sequence as set forth in SEQ ID NO:51 under stringent conditions.

The lectin encoded by the DNA comprising the base sequence as set forth in SEQ ID NO:51 is a precursor, and its mature type lectin is the lectin comprising the amino acid sequence as set forth in SEQ ID NO:50 or the like.

The “algMPL” according to the present invention is a lectin comprising the amino acid sequence as set forth in SEQ ID NO:73 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:73.

The “algCSA” according to the present invention is a lectin derived from a green alga (Codium subtubulosum) that is present in a fraction obtained by extracting the green algae with buffer, salting out the obtained soluble fraction, dialyzing the obtained precipitate, followed by adsorption of the precipitate on a column immobilized with submaxillary mucin and then elution with N-acetyl-D-galactosamine and that has a molecular weight of from 10,000 to 15,000 Da and displays an agglutination activity against trypsin-treated rabbit red blood cells.

Examples of the “algCSA” according to the present invention include a lectin obtained by the purification method below. Specifically, a green alga (Codium subtubulosum) is first lyophilized. This is added to a buffer (e.g., pH 7-8, Tris buffer or phosphate buffer), and after stirring (e.g., after stirring overnight at 4° C.), centrifugation recovers a supernatant. The obtained extract is added to an inorganic salt (e.g., ammonium sulfate, ammonium chloride, sodium sulfate, sodium chloride or potassium chloride) at stirring so that a predetermined saturation concentration (e.g., 70-80%) is attained. After stirring, standing (e.g., after stirring at 4° C. for further 30 min, standing is allowed to continue overnight) causes salting-out. This is centrifuged to obtain precipitates, and they are dissolved in a buffer (e.g., pH 7-8, Tris buffer or phosphate buffer) and are dialyzed against the buffer sufficiently. An internal solution is next recovered to prepare a salted out fraction. The obtained salted out fraction is added to a column immobilized with submaxillary mucin and it is eluted with N-acetyl-D-galactosamine after washing. Moreover, the absorbance at a wavelength of 280 nm and agglutination activity against trypsin-treated rabbit red blood cells are used as indicators to select the lectin, which is present in the eluted fraction by gel filtration: it is a typical example of the “algCSA” according to the present invention to be mentioned.

The forms of these lectins can be “natural lectins” that are obtained by the separation, extraction and purification from natural products such as plants, animals, microorganisms (e.g., bacteria and viruses). Amino acid sequences of proteins can vary in nature (i.e., non-artificially). Therefore, in the present invention such natural variants are also encompassed by the “natural lectins.”

The forms of such lectins can also be lectins (artificial lectins) that are synthesized by genetic techniques based on the gene sequences of natural lectins using cell-free protein synthesis systems (such as a reticulocyte extract or a wheat germ extract), E. coli, animal cells, insect cells, or plant cells. Furthermore, such “artificial lectins” may be those where modifications are artificially made to the amino acid sequences (such as partial fragments of a lectin containing a sugar chain binding site). The “artificial lectin” may be fused directly or indirectly to a functional protein. The functional protein is not particularly limited and is appropriately selected, depending on the function to be desirably imparted to the lectin according to the present invention. Examples of the functional protein that is used to facilitate the purification of the lectin according to the present invention include a Myc-tag protein, a His-tag protein, a hemagglutinin (HA)-tag protein, a FLAG-tag protein (the registered trademark of Sigma-Aldrich Inc.), and a glutathione-S-transferase (GST) protein.

The forms of such lectins may be dimers, multimers, fragmented ones by enzymatic digestion or the like (such as a lectin from which a signal peptide has been removed or a mature lectin produced from a precursor lectin (pro-lectin) by undergoing processing).

Furthermore, from the viewpoint that the genus Staphylococcus at the stationary phase can be distinguished from bacteria at the stationary phase other Staphylococci (Escherichia coli, Bacillus subtilis and Pseudomonas aeruginosa), the “lectin” according to the present invention is preferably HAA, HPA, LEL, STL, Tachylectin-2, BCL11 or ULL among those lectins.

Also, from the viewpoint that Staphylococcus aureus at the stationary phase can be distinguished from Staphylococcus capitis at the stationary phase, Staphylococcus aureus at the stationary phase can be distinguished from Staphylococcus epidermidis at the stationary phase, Staphylococcus capitis at the stationary phase can be distinguished from Staphylococcus epidermidis at the stationary phase, and the genus Staphylococcus can be distinguished from bacteria at the stationary phase other than Staphylococci (Escherichia coli, Bacillus subtilis, and Pseudomonas aeruginosa), the “lectin” according to the present invention is preferably Tachylectin-2 or LEL.

Also, from the viewpoint that Staphylococcus aureus at the logarithmic growth phase can be distinguished from Staphylococcus capitis at the logarithmic growth phase, Staphylococcus aureus at the logarithmic growth phase can be distinguished from Staphylococcus epidermidis at the logarithmic growth phase, and Staphylococcus capitis at logarithmic growth phase can be distinguished from Staphylococcus epidermidis at the logarithmic growth phase, the “lectin” according to the present invention is preferably proBCA1, proBCA2, UEA-II, RSL, CPA or CHA-1 among those lectins.

Furthermore, two or more types of lectins can be combined among those lectins for use in the present invention. For example, BCL11 capable of distinguishing Staphylococcus aureus at the stationary phase from Staphylococcus epidermis at the stationary phase, hypninA3 capable of distinguishing Staphylococcus aureus at the stationary phase from Staphylococcus capitis at the stationary phase, and WFL capable of distinguishing Staphylococcus capitis at the stationary phase from Staphylococcus epidermis at the stationary phase are combined for use; thereby, it will be possible to distinguishing from each other among the three types of bacteria at the stationary phases.

Also, according to the present invention, in the case where a species at the stationary phase within the genus Staphylococcus is targeted, the binding affinity to at least one lectin selected from the group consisting of Tachylectin-2, LEL, KAA1, BCL11, CBA, HAA, HPA, STL, proBCA1, proBCA2, ULL, DSA, PWM, UDA, WFL, hypninA3, BanLec, BML11b, BCL11d, Pro-CFA I, Pro-CFA II, CHA-1, FVE, Tachylectin-3, RSL, API 144, CLA, AC-avranin, UPL-1, BML11c, MPA1, and algCSA is preferably used as an indicator to distinguish between species at the stationary phase within the genus Staphylococcus; more preferably, all these lectins are used to distinguish between species at the stationary phase within the genus Staphylococcus.

Also, according to the present invention, in the case where a species at the logarithmic growth phase within the genus Staphylococcus is targeted, the binding affinity to at least one lectin selected from the group consisting of CBA, proBCA1, proBCA2, DSA, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, BPL, CFA1, CFA2, Pro-CFA II, MPA2, and algMPL is preferably used as an indicator to distinguish between the species at the logarithmic growth phase within the genus Staphylococcus; more preferably, all these lectins are used to distinguish between the species at the logarithmic growth phase within the genus Staphylococcus.

Also, according to the present invention, in the case where a species at the logarithmic growth phase within the genus Staphylococcus in milk is targeted, the binding affinity to at least one lectin selected from the group consisting of algMPL, PNA, GSL-II, BCL11, DBA, Tachylectin-3, TPL-1, BML11b, BML11c, Tachylectin-2, PVL, LBA, and UPL-1 is preferably used as an indicator to distinguish between the species at the logarithmic growth phase within the genus Staphylococcus in milk; more preferably, all these lectins are used to distinguish between the species at the logarithmic growth phase within the genus Staphylococcus in milk.

Furthermore, according to the present invention, it is particularly preferred that all of these Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, algMPL, and algCSA lectins be used to distinguish between species within the genus Staphylococcus.

In addition, as will be shown in the Examples to be described later, at least one lectin selected from the group consisting of GSL-II, PVL, and WGA can be used to distinguish the genus Staphylococcus at the stationary phase from bacteria at the stationary phase other than Staphylococci (Escherichia coli, Bacillus subtilis, and Pseudomonas aeruginosa); therefore, the lectin is also preferably used in combination with the lectins according to the present invention (for example, CBA, proBCA1, proBCA2, KAA1, DSA, PWM, UDA, WFL, or hypninA3).

Note that in the present invention, “WGA” is a lectin comprising the amino acid sequence as set forth in SEQ ID NO:17 or an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:17. Typical examples of the “WGA” according to the present invention include Wheat Germ Agglutinin (WGA) (manufactured by Vector Laboratories Inc.; Catalog No. L-1020) and WGA (Wheat Germ) lectin (manufactured by Seikagaku Corporation; Code No. 300191).

It is also preferable to use an antibody capable of distinguishing the genus Staphylococcus from bacteria other than Staphylococci in place of the aforementioned lectins, in combination with the lectin(s) according to the present invention. Examples of such antibody include the anti-Staphylococcus aureus murine monoclonal antibody (manufactured by LifeSpan BioSciences, Inc.; Catalog No. LS-C76000).

There is no particular limitation to the conditions under which the lectin according to the present invention is allowed to contact the above-described specimen and to bind to Staphylococci and others that may be contained in the specimen. However, from the viewpoint that the frequency of contact of the lectin(s) according to the present invention with Staphylococci and others which may be contained in the specimen is enhanced so as to facilitate their binding, the Staphylococci and others that may be contained in the specimen are preferably cultured. It is also preferred that the specimen be subjected to affinity purification using magnetic beads or the like immobilized with an antibody which specifically recognizes the Staphylococci and others or with the lectin and that the Staphylococci and others be concentrated.

Note that a “medium for culturing a specimen” and “magnetic beads wherein an antibody or lectin is immobilized” to be described later in <Kit for Distinguishing between Species within the Genus Staphylococcus> are preferably used for such culture or purification.

In addition, the conditions under which the lectin according to the present invention is allowed to contact the above-described specimen and to bind to Staphylococci and others that may be contained in the specimen are preferably such that the lectin(s) according to the present invention is immobilized on a substrate.

Materials of such substrate are not particularly limited. For example, there may be mentioned synthetic resins (such as nylon, polystyrene, polycarbonate, and polypropylene), silicas such as glass, metals (such as platinum, silver, copper and gold), silicone, mica and a mixture of the foregoing. The surface of the material may have been subjected to surface treatment so that the surface can be immobilized with the lectin(s) (such as maxisorp treatment, polysorp treatment, medisorp treatment, and multisorp treatment).

Further, the shapes of such substrate are not particularly limited; for example, plates, chips and beads are mentioned. A plurality of lectins, including the lectin(s) according to the present invention, may be immobilized on the substrate and may be utilized as an array in the method of the present invention. In this case, the lectins are preferably arranged and immobilized on the substrate so that clear identification patterns can be detected. Note that in case of one skilled in the art, the fabrication and utilization of such array can be accomplished by appropriately altering fabrication procedures or detection methods for known DNA chips or protein chips.

There is no particular limitation to the method of immobilizing the lectin according to the present invention on the substrate. For example, there may be mentioned methods utilizing physical adsorption, electrostatic interaction, hydrophobic interaction, cross-linking agents, antibodies that are specific to the lectin(s) according to the present invention.

Concentration at the time of immobilization of the lectin according to the present invention may be appropriately adjusted in accordance with the material or shape of the substrate, the method of immobilization, the binding affinity of the lectin for use and bacteria, the method of detecting the bacteria bound to the lectin or the like. For example, a concentration of from 1 to 10,000 μg/ml is mentioned, and the concentration is preferably from 5 to 20 μg/ml.

After immobilization of the lectin(s) according to the present invention, a variety of high polymers [such as dextran, polyethylene glycol, polylactic acid, polycarboxylate, and 2-methacryloyloxyethylphosphorylcholine (MPC)] can be used as blocking agents. Commercially available products of N101, N102, LIPIDURE (registered trademark; manufactured by NOF Corporation) and the like can preferably be used. Note that these blocking agents contribute to increasing stability of a substrate immobilized with the lectin(s), in addition to the prevention of non-specific adsorption. Amino acids such as glycine, saccharose or the like may also coexist during blocking.

The conditions under which the lectin according to the present invention is brought into contact with the specimen may be those which enable the binding between the lectin according to the present invention and Staphylococcus. For example, there may be mentioned bringing into contact at 0-4° C.; preferable is bringing into contact at 4-37° C. Also, in the preparation of the specimen, the pH of a liquid to dilute the bacterium is, for example, pH 6-8 to be mentioned. There can be preferably used buffers that will be later described in “Reagents for Diluting Specimen” according to the present invention. Furthermore, as the concentration of bacterium that is brought into contact with the lectin a turbidity of from 0.001 to 4 at a wavelength of 660 nm is mentioned, and a turbidity of from 0.1 to 1 is preferably mentioned.

There is no particular limitation to the method for detecting Staphylococcus that is bound to the lectin according to the present invention. Known detection method of Staphylococcus can appropriately be selected and utilized. As such method, there is, for example, mentioned a method where after staining with crystal violet and washing, the dye is allowed to flow out from Staphylococcus and others and its absorbance (wavelength: 570 nm) is measured. Further, there is mentioned a method by which the surface plasmon resonance phenomenon resulting from Staphylococcus being bound to the lectin immobilized on a metal thin film is measured by Biacore (manufactured by GE Healthcare). Also, there is mentioned a method where the number of bacteria is quantified by measuring Staphylococcus bound to the lectin that is arrayed on a microplate with a CCD camera or by labeling the Staphylococcus with a fluorescence reagent, such as Cy3 or Cy5, and measuring the fluorescence intensity. Furthermore, there are mentioned a method by which the lectin is immobilized on Luminex beads (registered trademark; Hitachi Solutions, Ltd) and a Luminex system (registered trademark; Hitachi Solutions, Ltd) is used for measurement according to Multiple Analyte Profiling, and a qualitative measuring method by an immunochromatography wherein the lectin is in the solid phase.

As described above, Staphylococcus may also be stained in order to detect or to facilitate the detection of the Staphylococcus bound to the lectin according to the present invention. Examples of the reagents to be used in such staining include crystal violet, sulforhodamine B (SRB), and fluorescent reagents such as DAPI, FITC, Cy3, and CY5. Further, from the viewpoint of detecting Staphylococcus with high specificity, examples of the reagents to be used in such detection include labeled antibodies and labeled lectins. As such label, it is possible to use radioactive substances, fluorescent dyes, chemiluminescent substances, enzymes, and co-enzymes, for example. Specifically, there are mentioned radioisotope, fluorescein, rhodamine, dansyl chloride, luciferase, peroxidase, alkaline phosphatase, lysozyme, and biotin/avidin. In addition, such antibodies and lectins may be those capable of specifically binding to the Staphylococcus which will be the subject of detection, and the lectins according to the present invention can preferably be used, for example.

Furthermore, the Staphylococcus bound to the lectin(s) according to the present invention may be immobilized with a cross-linking reagent before or after the detection of such Staphylococcus. Such cross-linking reagent is not particularly limited and the examples include cross-linking groups including glutaraldehyde, bismaleimidohexane, bis(sulfosuccinimidyl)suberate, m-maleimidobenzoyl-N-hydroxysuccinimide ester, succinimidyl 4-(maleimidomethyl)-cyclohexane-1-carboxylate.

Binding affinity between the lectin(s) according to the present invention and Staphylococcus may employ the numeric values obtained by the above-described methods (such as absorbance, bacterium number, and fluorescence intensity) themselves as indicators for distinguishing in the method of the present invention. The values having undergone logarithmic conversion or other numerical inversions can also be used. As will be shown in the Examples to be described later, the values calibrated from numeric values obtained using the lectin according to the present invention based on numeric values obtained in the absence of the lectin may be used; the values calibrated from numeric values obtained using the lectin according to the present invention based on numeric values obtained using the lectin for which reaction has been commonly confirmed among respective bacteria (such as GSL-II among the respective bacteria at the stationary phases) may be used.

There is no particular limitation to the method for distinguishing between species based on the binding affinity as an indicator. The lectin according to the present invention has different binding affinities to among species within the genus Staphylococcus; therefore, comparative distinguishing can be done by basing a binding affinity to at least one species within the genus Staphylococcus and comparing binding affinities to other species. Also, such “distinguishing” is not particularly limited. For example, a variety of statistical methods (such as t-test, dispersion analysis, analysis of variance, the Tukey-Kramer multiple comparison method, and the Dunnett's multiple comparison test) can be utilized to evaluate the presence or absence of significant differences in binding affinities that differ between the above-described species. Further, in the method of the present invention, when a plurality of the lectins according to the present invention are used, it is possible to distinguish by performing classification (cluster analysis) based on binding affinities of the respective lectins to species within the genus Staphylococcus, as shown, for example, in Japanese Unexamined Patent Application Publication No. 2009-148236. Note that such cluster analysis can be performed by appropriately selecting and utilizing a software such as TIGRmeV, NIA Array Analysis, Stalib-MULTI, MULTIV, NetLIB, ALN, MIXMOD, Cluster 3.0, or MeV V4.0. Also, in the method of the present invention, when a plurality of the lectins according to the present invention are used, it is possible to distinguish based on the radar chart of each species, as will be shown in the Examples to be described later.

<Agent for Distinguishing Between Species within the Genus Staphylococcus>

The agent for distinguishing between species within the genus Staphylococcus according to the present invention is characterized by comprising at least one lectin selected from the group consisting of Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MPA2, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, algMPL, and algCSA.

As will be shown in the Examples to be described later, the agent of the present invention can distinguish between species within the genus Staphylococcus; therefore, it can preferably be used as a reagent that is intended for use in the food hygiene inspection or the like in addition to as a diagnostic agent that is intended for use in the tests of patients having infection.

The agent of the present invention may comprises at least one lectin among the lectins according to the present invention but may comprises two or more of the lectins according to the present invention. There is no particular limitation to the method of preparing those lectins according to the present invention. For example, there are mentioned the respective preparation methods (separation, extraction and purification methods) described in <Method of Distinguishing between Species within the Genus Staphylococcus> and the lectin preparation methods described in <Lectins and DNAs Encoding the Lectins> to be described later.

The agent of the present invention can comprise other components that are acceptable agents, in addition to the lectin(s) according to the present invention. Examples of such additional components include a carrier, an excipient, a disintegrating agent, a buffering agent, an emulsifier, a suspending agent, a stabilizer, a preservative, an antiseptic, and physiological saline. As the excipient, there can be used lactose, starch, sorbitol, D-mannitol, sucrose, and the like. As the disintegrating agent, there can be used starch, carboxymethyl cellulose, calcium carbonate, and the like. As the buffering agent, there can be used a phosphate, a citrate, an acetate, and the like. As the emulsifier, there can be used gum arabic, sodium alginate, tragacantha, and the like. As the suspending agent, there can be used glyceryl monostearate, aluminum monostearate, methylcellulose, carboxymethyl cellulose, hydroxylethyl cellulose, sodium lauryl sulfate, and the like. As the stabilizer, there can be used propylene glycol, diethylene sulfite, ascorbic acid, and the like. As the preservative, there can be used phenol, benzalkonium chloride, benzyl alcohol, chlorobutanol, methylparaben, and the like. As the antiseptic, there can be used sodium azide, benzalkonium chloride, para-hydroxybenzonate, chlorobutanol, and the like.

<Kit for Distinguishing Between Species within the Genus Staphylococcus>

The kit for distinguishing between species within the genus Staphylococcus according to the present invention is characterized by comprising;
a substrate where there is immobilized at least one lectin selected from the group consisting of Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MPA2, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, algMPL, and algCSA; and
at least one reagent selected from the group consisting of:
(a) a reagent for detecting a specimen;
(b) a blocking reagent;
(c) a reagent for immobilizing the specimen; and
(d) a reagent for diluting the specimen.

As will be shown in the Examples to be described later, the kit of the present invention can distinguish between species within the genus Staphylococcus; therefore, it can preferably be used as a kit that is intended for use in the food hygiene inspection or the like, in addition to as a kit that is intended for use in the tests of patients having infection.

The substrate on which the lectin is immobilized according to the present invention can, for example, be prepared by appropriately selecting the material, arrangement, immobilization method, concentration at the time of immobilization for the substrate or the like, as described in

<Method of Distinguishing Between Species within the Genus Staphylococcus>.

The “reagent for detecting a specimen” according to the present invention may be one capable of detecting the “species within the genus Staphylococcus” which may be contained in the specimen. The examples include crystal violet, sulforhodamine B (SRB), fluorescence reagents such as DAPI, FITC, Cy3 and Cy5, the above-described labeled antibodies and the above-described labeled lectins.

The “blocking agent” according to the present invention may be one capable of suppressing non-specific adsorption to the substrate according to the present invention. The examples include high polymers such as dextran, polyethylene glycol, polylactic acid, polycarboxylate, 2-methacryloyloxyethylphosphorylcoline (MPC). In addition, the “blocking agent” according to the present invention may be that which contains an amino acid such as glycine, sucrose or the like.

The “reagent for immobilizing the specimen” according to the present invention may be one capable of cross-linking the “species within the genus Staphylococcus” which may be contained in the specimen with the lectin(s) according to the present invention. The examples include glutaraldehyde, bismaleimidohexane, bis(sulfosuccinimidyl)suberate, m-maleimidobenzoyl-N-hydroxysuccinimide ester, succinimidyl 4-(maleimidomethyl)-cyclohexane-1-carboxylate.

The “reagent for diluting the specimen” according to the present invention may be that which does not inhibit binding between the “species within the genus Staphylococcus” which may be contained in the specimen and the lectin(s) according to the present invention. The examples include buffers (pH 6-8), more specifically, a Tris buffer, aphosphate buffer, a citrate buffer, a HEPES buffer, a MES buffer, a Bis-Tris buffer, and a MOPS buffer. In addition, these buffers may contain salts, surfactants, proteins, sugars, zwitterionic compounds and the like, as appropriate.

These salts are not particularly limited but are preferably salts that cause cations to be formed in buffers. The examples include calcium chloride (calcium ion), manganese chloride (manganese ion), and magnesium chloride (magnesium ion).

These surfactants are not particularly limited but are preferably nonionic surfactants. The examples include Tween-20 and Triton X-100.

These proteins are not particularly limited but are preferably those which act as stabilizers or blocking agents. The examples include bovine serum albumin, gelatin, and casein.

These sugars are not particularly limited but are preferably those which act as stabilizers or blocking agents. The examples include sucrose, trehalose, mannitol, sorbitol, galactose, glucose, mannose, xylose, N-acetyl-D-glucosamine, N-acetyl-D-galactosamine, fucose, N-acetylneuraminic acid, N-glycolylneuraminic acid, deaminoneuraminic acid (2-keto-3-deoxy-D-glycero-D-galacto-nononic acid; KDN), glucuronic acid, iduronic acid, lactose, chitobiose, and chitotriose.

These zwitterionic compounds are not particularly limited but are preferably those which act as stabilizers or blocking agents. The examples include betaine, taurine, arginine, glycine, lysine, and histidine.

The kit for distinguishing between species within the genus Staphylococcus according to the present invention may also contain, other than the above-described substrate and others, other reagents that can be used in the method for distinguishing between species within the genus Staphylococcus according to the present invention. Such other reagents include a medium for culturing a specimen, magnetic beads immobilized with antibodies or lectins, a cleaning solution, a positive control, and a negative control.

Such “medium for culturing a specimen” may be one capable of growing the “species within the genus Staphylococcus” and others which may be contained in the specimen. Examples include a mannitol salt medium, a mannitol salt medium supplemented with egg york, Staphylococcus Medium No. 110, a Baird Parker medium, buffered peptone water, an LB medium, a heart infusion medium, a brain heart infusion medium, a tryptic soy medium, a standard agar medium, a nutrient agar medium, and a blood agar medium.

Such “magnetic beads immobilized with antibodies or lectins” may be magnetic beads wherein there are immobilized antibodies or lectins capable of specifically binding to the “species within the genus Staphylococcus” and others that may be contained in the specimen. The examples include an anti-protein A antibody and magnetic beads wherein the lectin (s) according to the present invention or WGA is immobilized.

The “cleaning solution” may be one that does not inhibit binding between the “species within the genus Staphylococcus” and others that may be contained in the specimen and the lectin(s) according to the present invention and that is capable of cleaning bacteria and fluorescent dyes or others which are non-specifically adsorbed on the substrate or the like. The examples include the “reagent for diluting the specimen” described above.

As the “positive control” and the “negative control,” there are, for example, mentioned specific species within the genus Staphylococcus that are targeted by the detection and bacterial species that are different from said species, respectively.

The kit for distinguishing between species within the genus Staphylococcus also may comprise an “enzyme substrate solution” that is allowed to react with a label and to generate chemiluminescence when the labeled antibody or the labeled lectin is used as the reagent for detecting a specimen or may comprise a “stop solution” to terminate the reaction.

The kit of the present invention can also include a user's manual of the substrate and the like in practicing the method of the present invention.

<Lectins and DNAs Encoding the Lectins>

The present invention provides at least one lectin selected from the group consisting of (a) to (c) below:

(a) a lectin comprising the amino acid sequence as set forth in SEQ ID NO:3;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:3; and
(c) a lectin encoded by a DNA that hybridizes with a DNA comprising the base sequence as set forth in SEQ ID NO:34 under stringent conditions.

The present invention provides at least one lectin selected from the group consisting of (a) to (c) below:

(a) a lectin comprising the amino acid sequence as set forth in SEQ ID NO:4;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:4; and
(c) a lectin encoded by a DNA that hybridizes with a DNA comprising the base sequence as set forth in SEQ ID NO:35 under stringent conditions.

The present invention provides at least one lectin selected from the group consisting of (a) to (c) below:

(a) a lectin comprising the amino acid sequence as set forth in SEQ ID NO:13;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:13; and
(c) a lectin encoded by a DNA that hybridizes with a DNA comprising the base sequence as set forth in SEQ ID NO:36 under stringent conditions.

The present invention provides at least one lectin selected from the group consisting of (a) to (c) below:

(a) a lectin comprising the amino acid sequence as set forth in SEQ ID NO:14;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:14; and
(c) a lectin encoded by a DNA that hybridizes with a DNA comprising the base sequence as set forth in SEQ ID NO:37 under stringent conditions.

The present invention provides at least one lectin selected from the group consisting of (a) to (c) below:

(a) a lectin comprising the amino acid sequence as set forth in SEQ ID NO:38;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:38; and
(c) a lectin encoded by a DNA that hybridizes with a DNA comprising the base sequence as set forth in SEQ ID NO:39 under stringent conditions.

The present invention provides at least one lectin selected from the group consisting of (a) to (c) below:

(a) a lectin comprising the amino acid sequence as set forth in SEQ ID NO:40;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:40; and
(c) a lectin encoded by a DNA that hybridizes with a DNA comprising the base sequence as set forth in SEQ ID NO:41 under stringent conditions.

The present invention provides at least one lectin selected from the group consisting of (a) to (c) below:

(a) a lectin comprising the amino acid sequence as set forth in SEQ ID NO:42;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:42; and
(c) a lectin encoded by a DNA that hybridizes with a DNA comprising the base sequence as set forth in SEQ ID NO:43 under stringent conditions.

The present invention provides at least one lectin selected from the group consisting of (a) to (c) below:

(a) a lectin comprising the amino acid sequence as set forth in SEQ ID NO:44;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:44; and
(c) a lectin encoded by a DNA that hybridizes with a DNA comprising the base sequence as set forth in SEQ ID NO:45 under stringent conditions.

The present invention provides at least one lectin selected from the group consisting of (a) to (c) below:

(a) a lectin comprising the amino acid sequence as set forth in SEQ ID NO:46;
(b) a lectin comprising an amino acid sequence having a homology of 90% or more (e.g., 91% or more, 92% or more, 93% or more, 94% or more, 95% or more, 96% or more, 97% or more, 98% or more, and 99% or more) to the amino acid sequence as set forth in SEQ ID NO:46; and
(c) a lectin encoded by a DNA that hybridizes with a DNA comprising the base sequence as set forth in SEQ ID NO:47 under stringent conditions.

The present invention also provides a lectin derived from a green alga (Avrainvillea captituliformis), the lectin being present in a fraction obtained by extracting the green algae with buffer, salting out an obtained soluble fraction, dialyzing an obtained precipitate and purifying the precipitate with gel filtration, as well as having a molecular weight of from 15,000 to 20,000 Da as shown in reductive SDS-PAGE and displaying an agglutination activity against trypsin-treated rabbit red blood cells.

The present invention further provides a lectin derived from a green alga (Codium subtubulosum), the lectin being present in a fraction obtained by extracting the green algae with buffer, salting out a obtained soluble fraction with ammonium sulfate, dialyzing an obtained precipitate, followed by adsorption of the precipitate on a column immobilized with submaxillary mucin and then elution with N-acetyl-D-galactosamine, as well as having a molecular weight of from 10,000 to 15,000 Da and displaying an agglutination activity against trypsin-treated rabbit red blood cells.

The present invention also provides lectins in the forms of these lectins being additionally fused to functional proteins. In such forms, a functional protein can be directly or indirectly fused to either one or both of the N-terminus and C-terminus of the lectin, and/or between a signal sequence or the like and a matured lectin sequence. In the case where the functional protein and the lectin are indirectly fused, fusion can be done through a linker peptide. The sequence and length of such linker peptide are not particularly limited, and normally, there is mentioned a polypeptide comprising 1-50 amino acids, preferably 1-30 amino acids, and more preferably 1-20 amino acids. There is no particular limitation to the functional protein and it can be appropriately selected depending on the function to be desirably imparted to the lectin. Examples of the functional protein that is used to facilitate the purification of the lectin include an Myc-tag protein, a His-tag protein, a hemagglutinin (HA)-tag protein, a FLAG-tag protein (the registered trademark of Sigma-Aldrich Inc.), and a glutathione-S-transferase (GST) protein. Examples of the functional protein that is used to facilitate the detection of the lectin include green fluorescent protein (GFP) and luciferase.

Further, it is possible to prepare the lectin as well as to prepare the above-described lectin to which the functional protein is fused by inserting a DNA encoding each lectin into a suitable expression vector, introducing the vector into a cell-free protein synthesis system (such as a reticulocyte extract or a wheat germ extract) followed by incubation, or by introducing the vector into suitable cells and culturing the obtained transformant, to purify the expressed protein. Accordingly, the present invention provides DNAs encoding any one lectin of these lectins.

In addition, according to the present invention with respect to lectins whose concrete amino acid sequences or base sequences have not been obtained (the lectin derived from a green alga—Avrainvillea captituliformis and the lectin derived from a green alga—Codium subtubulosum), an amino acid analyzer [such as Procise494 (registered trademark; manufactured by Applied Biosystems Inc.) and PPSQ-31A/33A (manufactured by Shimadzu Corporation)] is used to sequence amino acid sequences in the N-terminal regions of the green algae-derived lectins, after having performed separation with electrophoresis or peptide purification with reverse phase HPLC as necessary. Also, a mass spectrometer (such as MALDI-TOFMS and LC-MS/MS) can be used to sequence an arbitrary sequence in the green algae-derived lectins. Furthermore, it is possible to prepare DNAs encoding the green algae-derived lectins by designing degenerate primers based on the thus-determined amino sequences and using the full-length cDNAs derived from the green algae as templates to carry out RACE, for example, as will be shown in the Examples to be described later.

Further, in the present invention, it is possible to prepare DNAs for encoding lectins comprising amino acid sequences that have homologies of 90% or more to amino acid sequences of natural lectins (such as the amino acid sequence as set forth in SEQ ID NO:3) by utilizing hybridization technologies which are known to one skilled in the art (such as a method described in Hanahan, D. et al., Meth. Enzymol., 1983, Vol. 100, pp. 333-342; a method described in Benton, W. D., et al., Science, 1977, pp. 180-182). Specifically, it is possible for one skilled in the art to carry out hybridization utilizing a DNA encoding a natural lectin (such as a DNA comprising the coding region of the base sequence as set forth in SEQ ID NO:34) or a portion thereof, to isolate from various organisms, DNAs having high homologies thereto, and to select a DNA encoding the lectin and comprising the amino acid sequence having a homology of 90% or more to the amino acid sequence of the natural lectin.

Furthermore, it is possible for one skilled in the art to prepare from various organisms, DNAs that hybridizes with DNAs comprising the base sequences of DNAs encoding the natural lectins (such as a DNA comprising the coding region of the base sequence as set forth in SEQ ID NO:34) under stringent conditions by utilizing DNAs encoding the natural lectins or portions thereof and carrying out the hybridization under the above-described “stringent conditions.”

Moreover, it is also possible to prepare DNAs encoding the lectins comprising amino acid sequences that have homologies of 90% or more to the amino acid sequences of natural lectins, by utilizing gene amplification technologies or gene manipulation technologies which are known to one skilled in the art, such as polymerase chain reaction (PCR) and site-directed mutagenesis (Kramer, W. & Fritz, H J., Methods Enzymol., 1987, 154, 340).

Further, it is possible for one skilled in the art to prepare the lectins of the present invention by selecting known techniques as appropriate, using the DNAs of the present invention. When the host cell (the aforementioned suitable cell) is Escherichia coli, examples of such known techniques include methods that use plasmid vectors pET-3 [Rosenberg, A. H. et al., Gene, 56, 125-35 (1987)] and pGEX-1 [Smith, D. B. and Johnson, K. S., Gene, 67, 31-40 (1988)]. Additionally, as the method for transforming Escherichia coli, there are mentioned a heat shock method (such as the calcium chloride method, Hanahan method, Inoue method, and rubidium chloride method), electroporation, and others.

When the host is a fission yeast (Schizosaccharomyces pombe), there is mentioned a method using plasmid vector pESP-1 [Lu, Q. et al., Gene, 200, 135-144 (1997)]. The method for transforming the yeast, for example, includes the spheroplast method, lithium acetate method, and electroporation.

Further, when the host is an insect cell, there is mentioned a method using a baculovirus vector pBacPAK8/9 (BD Clontech Co. Ltd.) or the like. Furthermore, the transformation of the insect cells can be, for example, carried out by a method described in Bio/Technology 6, 47-55 (1980).

When the host is a mammalian cell (such as CHO cells and HeLa cells), there is mentioned a method using a vector such as pMSG (BD Clontech Co. Ltd.). In addition, the introduction of recombinant DNAs to the mammalian cells can be carried out by the calcium phosphate method [Graham, F. L. and van derEb, A. J., Virology, 52, 456-467 (1973)], DEAE-dextran method [Sussman, D. J. and Milman, G., Mol. Cell. Biol., 4, 1641-1643 (1984)], lipofection method [Felgner, P. L. et al., Proc. Natl. Acad. Sci. USA, 84, 7413-7417 (1987)], electroporation [Neumann, E. et al., EMBO J., 1, 841-845 (1982)] or the like

Moreover, the recombinant proteins expressed in the host cells can be purified by the known methods; for example, there is mentioned an affinity chromatographic purification method using antibodies that specifically recognize the lectin(s) of the present invention. Note that it is possible for one skilled in the art to prepare the antibodies that specifically recognize the lectin(s) of the present invention by appropriately selecting techniques which are known. Such known techniques include a method for recovering the serum (polyclonal antibody) of an animal after having inoculated the lectin(s) of the present invention to the animal to be immunized and activated the immune system of the animal, the hybridoma method, the recombinant DNA method, and phage display.

Also, as the known method for purifying recombinant proteins expressed in host cells, there is, for example, mentioned a method by which the lectin is synthesized in a form of being fused to a functional protein such as a HIs-tag protein, a glutathione-S-transferase (GST) protein or the like and is purified by being bound to a metal chelate resin or a GST affinity resin [Smith, M. C. et al., J. Biol. Chem., 263, 7211-7215 (1988)]. Furthermore, it is possible to cleave between the functional protein and the lectin with thrombin, blood coagulation factor Xa or the like and to thereby separate only the lectin.

### EXAMPLES

Hereinafter, the present invention will be described more specifically on the basis of Examples. However, the present invention is not to be limited to the following Examples.

### Example 1

### Screening of Lectins Capable of Binding to Bacteria at Stationary Phase

<Lectins Used>

Screening of lectins was carried out using commercially available lectins, purified lectins from natural extracts, and recombinant lectins. The lectins used will be described below. Commercially available lectins:

### (Manufactured by EY Laboratories, Inc.)

AAA, ACA, AMA, APP, ASA, BDA, CA, CAA, calsepa, CCA, CPA, CSA, GHA, GS-IA4, GS-II, HMA, HPA, IRA, LAA, LBA, LFA, LPA, MIA, MNA-G, MNA-M, MOA, MPA, PAA, PMA, PSL, PTA-Gal, PTA-GalNAc, RPA, SHA, STA, TKA, TL, UDA, UEA-II, VFA, VRA, and WFA.

### (Manufactured by Vector Laboratories Inc.)

### EEL, GNL, GSL-I, GSL-IB4, GSL-II, HHL, Jacalin, LEL, MAH, MAL, MPL, NPL, PTL-I, PTL-II, RCA-I, Sc-WGA, SJA, SNA, STL, and WFL.

### (Manufactured by Seikagaku Corporation)

AAL, ABA, anti-H, ConA, DBA, DSA, ECA, GNA, LCA, Lotus, PHA-E4, PHA-L4, PNA, PSA, PWM, SBA, SSA, TJA-I, TJA-II, UEA-I, and WGA.

### (Manufactured by Sigma-Aldrich Corporation)

### CFL, HAA, and PA-I

### (Manufactured by Wako Pure Chemical Industries, Ltd.)

### PVL

Purified lectins from natural extracts:
CBA, algCPA, BCA, BCL11, milklectin
Recombinant lectins:
rACG, rproBCA1, rBCA1, rproBCA2, rBCA2, rBCL11, rCV-N, rKAA1, rGRFT, rhypninA1, rhypninA3, rMVL, rMVN, rOAA, rPA-IIL, rTachylectin-2, rTDA, rTPL-1, rTPL-2, and rULL. Note that the “r” of the “recombinant lectins” is affixed to the fronts of the names of lectins for the purpose of indicating recombinant proteins.

As described above, the commercially available lectins were purchased from EY Laboratories, Inc., Vector Laboratories Inc., Seikagaku Corporation, Sigma-Aldrich Corporation, or Wako Pure Chemical Industries, Ltd. and were used in the present Examples. The purified lectins from natural extracts were prepared at Hiroshima University or GLYENCE Co., Ltd. and were used. The recombinant lectins were constructed at GLYENCE Co., Ltd. and were used.

Note that the GS-II manufactured by EY Laboratories, Inc. and the GSL-II manufactured by Vector Laboratories Inc. are identical lectins, except that their manufacturers are different.

<Strains Used>

The strains which were used in the present Examples are shown in Table 4. There were used two strains of Staphylococcus aureus as food poisoning bacteria, two strains of skin resident Staphylococci, Staphylococcus epidermidis and Staphylococcus capitis as resident bacteria, each one strain of Escherichia coli, Bacillus subtilis, and Pseudomonas aeruginosa as a bacterium other than Staphylococci. The respective strains were purchased from The American Type Culture Collection (ATCC) The details of each strain are shown in Table 4.

TABLE 4

| Bacteria | ATCC No. | Information | |
|---|---|---|---|
| Staphylococcus aureus subsp. aureus | 6538 | food poisoning bacterium | Gram positive coccus |
| Staphylococcus aureus subsp. aureus | 27217 | food poisoning bacterium | Gram positive coccus |
| Staphylococcus epidermidis | 12228 | resident bacterium | Gram positive coccus |
| Staphylococcus epidermidis | 14990 | resident bacterium | Gram positive coccus |
| Staphylococcus capitis subsp. capitis | 27840 | resident bacterium | Gram positive coccus |
| Staphylococcus capitis subsp. capitis | 35661 | resident bacterium | Gram positive coccus |
| Escherichia coli | 8739 | reference bacterium | Gram negative bacillus |
| Bacillus subtilis subsp. Spizizenii | 6633 | reference bacterium | Gram positive bacillus |
| Pseudomonas aeruginosa | 9027 | reference bacterium | Gram negative bacillus |

<Lectin Screening by Plate Centrifugation>

Each 100 μl of the 119 types of lectins that had been diluted to 10 μg/ml with a carbonate buffer (pH 9.6) was sensitized overnight at 4° C. on a microplate (manufactured by Nunc A/S; surface-treatment maxisorp; Catalog No. 445101), and the lectins were immobilized on the microplate. Next, the lectin solution was removed, to which 300 μl of a blocking reagent for immunoassay N101 (manufactured by NOF Corporation) that had been diluted five times was added. Blocking was conducted at room temperature for 3 h.

Each strain shown in Table 4 was allowed to stand or was shake-cultured in a Todd-Hewitt medium at 37° C. for 24 h. The culture in which each bacterium reached a state of the stationary phase was washed with PBS three times; and 100 μl of a bacterial suspension that was prepared with 1% BSA/TBS-CM (TBS, 1% BSA, 1 mM CaCl₂, and 1 mm MnCl₂) so as to provide a turbidity (absorbance) of 1 at a wavelength of 660 nm was added to the plate. Centrifugation was carried out with a centrifuge at 4° C. and 510×g for 10 min. After centrifugation, 250 μl of TBS-CM (TBS, 1% BSA, 1 mM CaCl₂, and 1 mm MnCl₂) was gently added to the plate, which was sealed with a plate seal (manufactured by Nunc A/C; Catalog No. 236366). The plate was then inverted and centrifuged with a centrifuge at 4° C. and 160×g for 5 min. After centrifugation, 250 μl of a supernatant was removed from the plate, to which 100 μl of a TBS-CM/0.5% glutaraldehyde solution was added and fixed at room temperature for 1 h. After the glutaraldehyde solution was removed, the plate was washed with PBS and there was added 100 μl of 2.3% crystal violet. Staining was carried out at room temperature for 1 h and washing was done with running water. Thereafter, there was added 100 μl of 99.5% ethanol and the dye was eluted at room temperature for 1 h. Absorbance at 570 nm was quantified with a plate reader (manufactured by THERMO ELECTRON Co., Ltd; product name of Multiskan JX). Note that since the time required for such detention was on the order of 3.5 h, the method of the present invention was shown to be a method which can be performed rapidly and conveniently.

Further, as described above, the plate immobilized with the 112 types of lectins (lectin solid phase plate) was used to examine the binding of 9 types of bacteria to the lectins by the plate centrifugation. Note that the bacteria bound to the lectin solid phase plate can be stained with crystal violet and absorption can be seen at 570 nm. Absorbance of lectin GSL-II for which reaction was commonly observed among the respective bacteria was used as an indicator; and scattering between plates of the same bacterium was corrected to obtain data, which was shown as a bar graph. Specifically, the absorbance of GSL-II lectin was designated 100 and the correction was performed according to the equation below.

Correction value of each lectin (Index)=(mean value of absorbance of each lectin−mean value of absorbance of blank)×100/mean value of absorbance of GSL-II

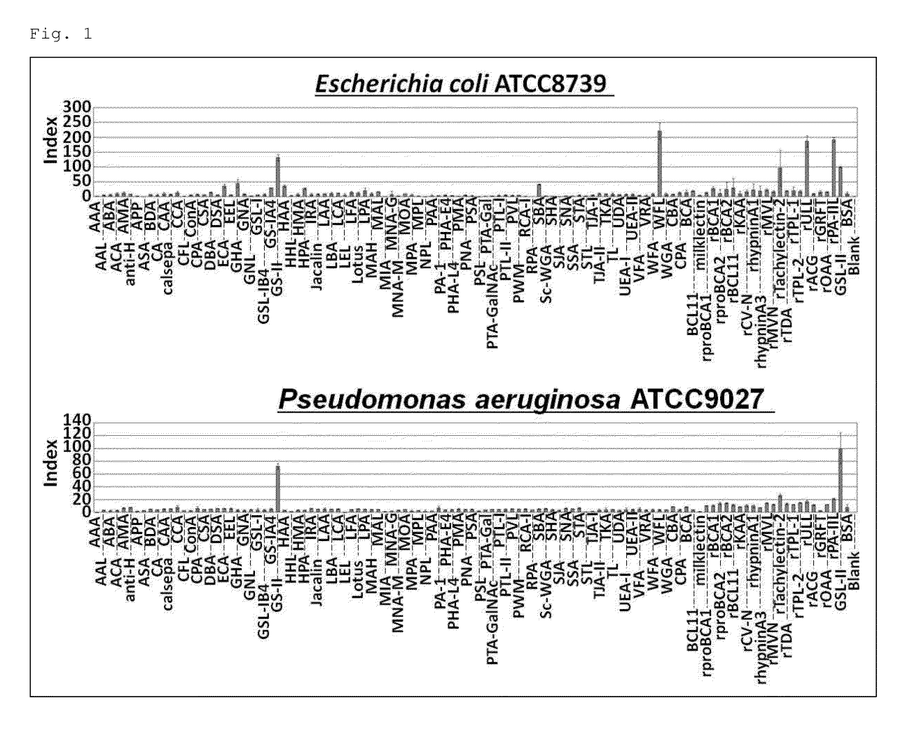

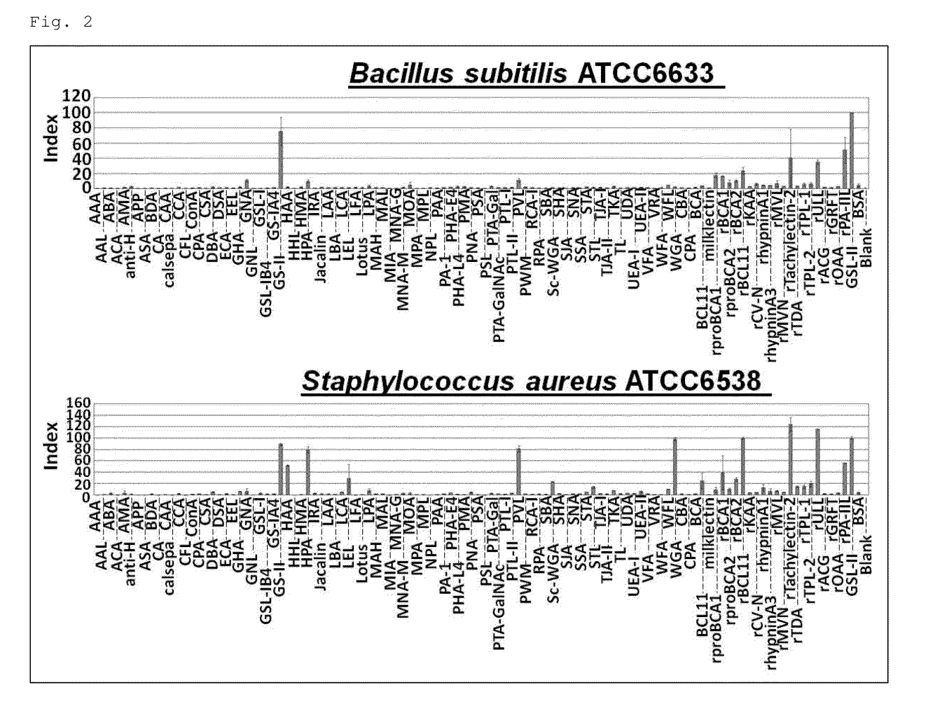

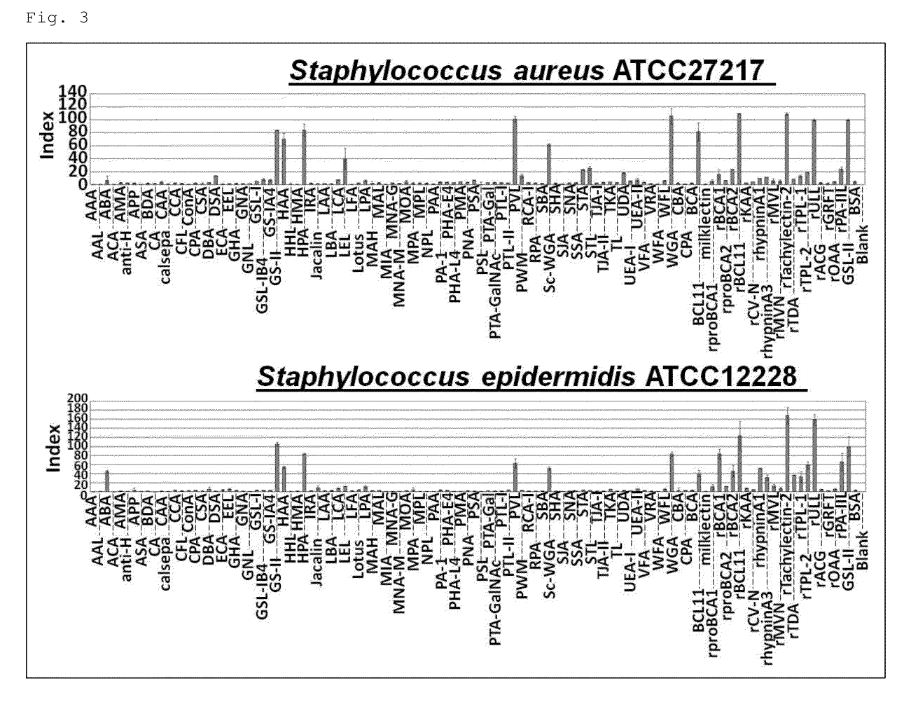

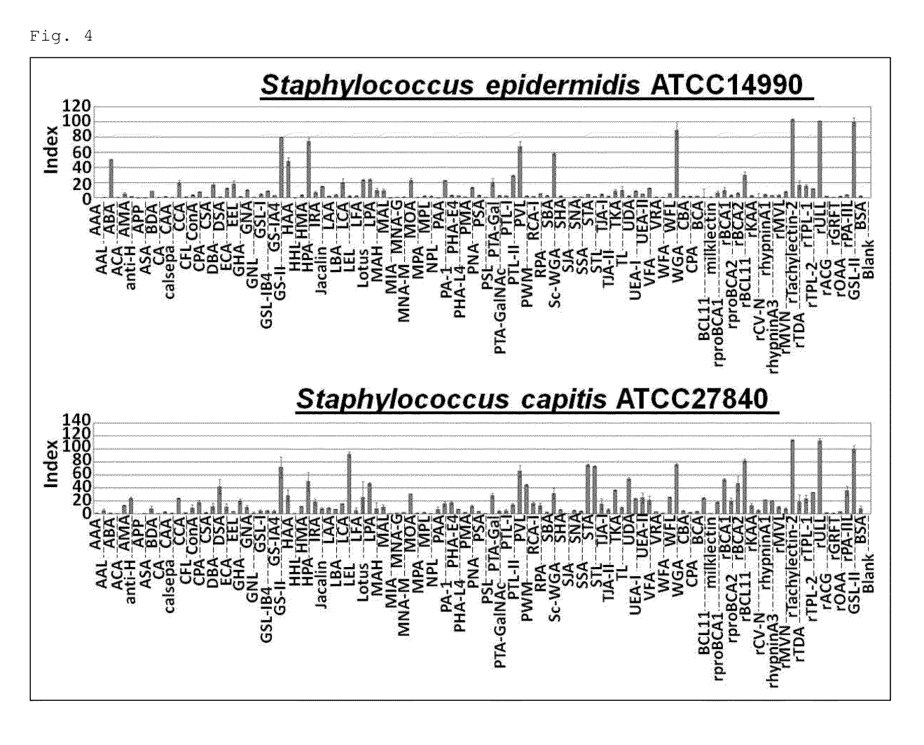

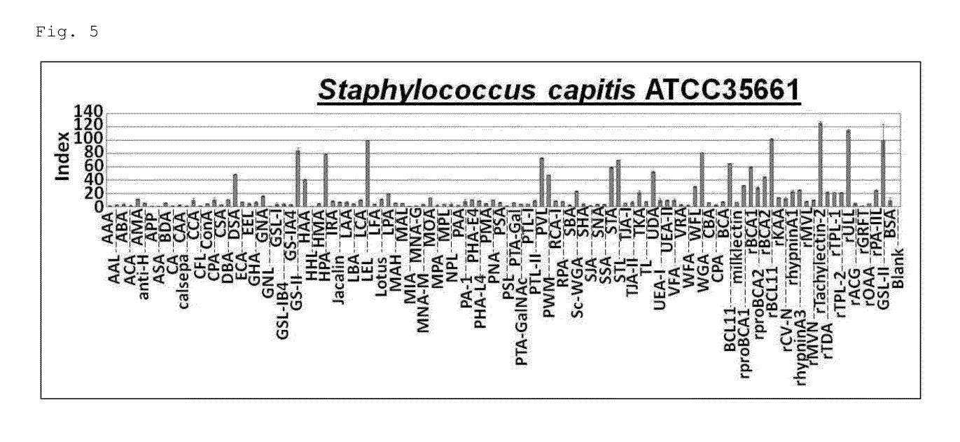

The obtained results are shown in FIG. 1 to 5.

Note that in FIGS. 1 to 5, “BSA” represents bovine serum albumin and “Blank” represents the absence of lectin. Also, “rKAA” represents the results of rKAA1.

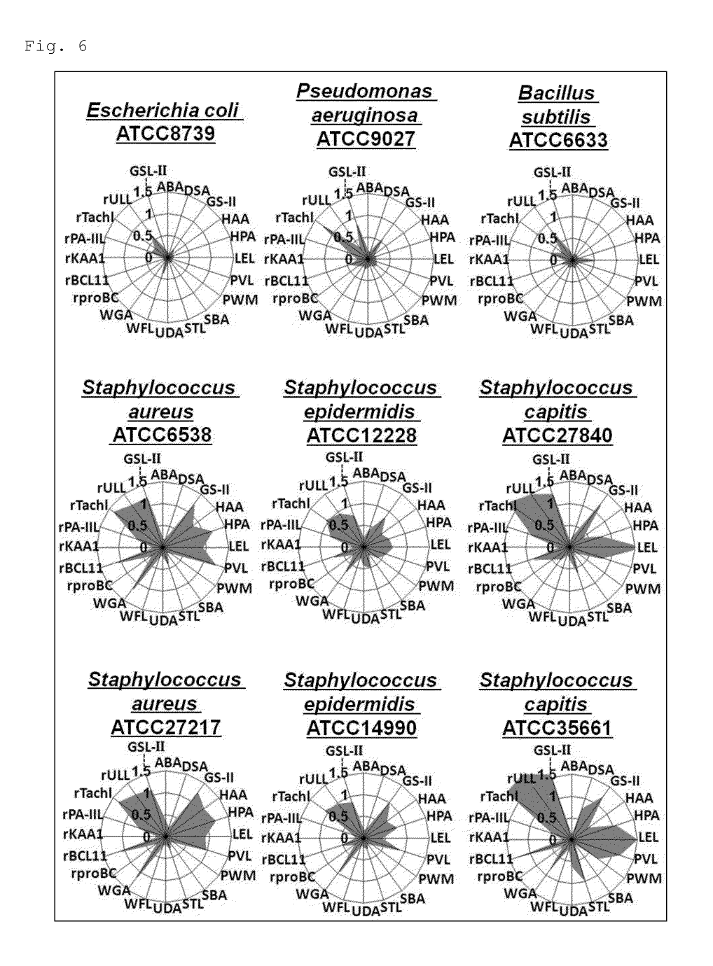

Next, 20 types of lectins, ABA, DSA, GS-II/GSL-II, HAA, HPA, LEL, PVL, PWM, SBA, STL, UDA, WFL, WGA, rproBCA2, rBCL11, rKAA1, rPA-IIL, rTachylectin-2 and rULL, were selected among the 112 types of lectins; and the binding of the 9 types of bacteria to the lectins was obtained by the plate centrifugation, which was shown in radar charts. The results are shown in FIG. 6. Note that in FIG. 6, “rTachy” represents the results of rTachylectin-2, “rproBCA” represents the results of rproBCA2, and “rKAA” represents the results of rKAA1.

As is evident from the results shown in FIGS. 1-6, large differences in the types of lectins to be bound were observed between the genus Staphylococcus and the other bacteria. Differences in the types of lectins to be bound were also observed between species even within the genus Staphylococcus.

### Example 2

### Selection of Lectins Capable of Distinguishing Between Staphylococcus aureus at Stationary Phase and Staphylococcus epidermidis at Stationary Phase

The above-described absorbance data was used to perform the significance test, and the lectins capable of distinguishing between Staphylococcus aureus at the stationary phase and other Staphylococcus (a skin resident Staphylococcus; Staphylococcus epidermidis) at the stationary phase were selected. The obtained results together with the conditions of the significance test are shown in Table 5. Note that, in examples 2-8, the statistical differences were confirmed by Student's t-test (two-sided test). In cases where unequal variances were suspected by F-test, Welch's t-test (two-sided test) was performed. Further, “P<0.05” was determined to be statically significant.

TABLE 5
Analysis Conditions

| Type of Test: | T test | |
|---|---|---|
| S.aureus (Group 1) | S.aureus ATCC 6538 | S.aureus ATCC 27217 |
| S.epidermidis (Group 2) | S.epidermidis ATCC 12228 | S.epidermidis ATCC 14990 |

| significance level: | 0.05 ns no significant difference *: p < 0.05 **: p < 0.01 ***: p < 0.001 | | | | | |
|---|---|---|---|---|---|---|
| | Group 1 | Group 1 | Group 2 | Group 2 | | |
| | Mean | Standard | Mean | Standard | | |
| Lectins | Value | Deviation | Value | Deviation | T value | P value |
| LEL | 0.5900 | 0.0283 | 0.1572 | 0.0534 | 10.1297 | 0.0096** |
| STL | 0.3302 | 0.0251 | 0.1178 | 0.0152 | 10.2402 | 0.0094** |
| rTachylectin-2 | 1.3468 | 0.0357 | 1.0590 | 0.0290 | 8.8473 | 0.0125* |
| rULL | 1.1815 | 0.0113 | 0.9440 | 0.0283 | 11.0257 | 0.0081** |
| rBCL11 | 1.3123 | 0.1729 | 0.6035 | 0.1358 | 4.5597 | 0.0449* |

As is evident from the results shown in Table 5, it was possible to distinguish between Staphylococcus aureus at the stationary phase and the other Staphylococcus (Staphylococcus epidermidis) at the stationary phase by using LEL, STL, Tachylectin-2, BCL11, or ULL.

### Example 3

### Selection of Lectins Capable of Distinguishing Between Staphylococcus aureus at Stationary Phase and Staphylococcus capitis at Stationary Phase

The above-described absorbance data was used to perform the significance test, and the lectins capable of distinguishing between Staphylococcus aureus at the stationary phase and other Staphylococcus (a skin resident Staphylococcus; Staphylococcus capitis) at the stationary phase were selected. The obtained results together with the conditions of the significance test are shown in Table 6.

TABLE 6
Analysis Conditions

| Type of Test: | T test | |
|---|---|---|
| S.aureus (Group 1) | S.aureus ATCC 6538 | S.aureus ATCC 27217 |
| S.capitis (Group 2) | S.capitis ATCC 27840 | S.capitis ATCC 35661 |

| significance level: | 0.05 ns no significant difference *: p < 0.05 **: p < 0.01 ***: p < 0.001 | | | | | |
|---|---|---|---|---|---|---|
| | Group 1 | Group 1 | Group 2 | Group 2 | | |
| | Mean | Standard | Mean | Standard | | |
| Lectins | Value | Deviation | Value | Deviation | T value | P value |
| HAA | 0.9688 | 0.0449 | 0.5067 | 0.0187 | 13.4287 | 0.0055** |
| LEL | 0.5900 | 0.0283 | 1.2735 | 0.1789 | 5.3369 | 0.0334* |
| rTachylectin-2 | 1.3468 | 0.0357 | 1.7830 | 0.0912 | 6.2982 | 0.0243* |
| DSA | 0.1985 | 0.1039 | 0.6463 | 0.0626 | 5.2190 | 0.0348* |
| PWM | 0.1698 | 0.0506 | 0.6655 | 0.1230 | 5.2706 | 0.0342* |
| rhypninA3 | 0.1890 | 0.0113 | 0.2990 | 0.0148 | 8.3331 | 0.0141* |

As is evident from the results shown in Table 6, it was possible to distinguish between Staphylococcus aureus at the stationary phase and the other Staphylococcus (Staphylococcus capitis) at the stationary phase by using HAA, LEL, Tachylectin-2, DSA, PWM, or hypninA3.

### Example 4

### Selection of Lectins Capable of Distinguishing Between Staphylococcus epidermidis at Stationary Phase and Staphylococcus capitis at Stationary Phase

The above-described absorbance data was used to perform the significance test, and the lectins capable of distinguishing between species of skin resident Staphylococci, namely Staphylococcus epidermidis at the stationary phase and Staphylococcus capitis at the stationary phase were selected. The obtained results together with the conditions of the significance test are shown in Table 7.

TABLE 7
Analysis Conditions

| Type of Test: | T test | |
|---|---|---|
| S. epidermis (Group 1) | S. epidermidis ATCC 12228 | S.epidermidis ATCC 14990 |
| S. capitis (Group 2) | S. capitis ATCC 27840 | S.capitis ATCC 35661 |

| | |
|---|---|
| signigicance level: | 0.05 ns: no significant difference *: p < 0.05 **: p < 0.01 ***: p < 0.001 |

| | Group 1 | Group 1 | Group 2 | Group 2 | | |
|---|---|---|---|---|---|---|
| | Mean | Standard | Mean | Standard | | |
| Lectins | Value | Deviation | Value | Deviation | T value | P value |
| CBA | 0.0997 | 0.0011 | 0.1330 | 0.0035 | 12.7391 | 0.0061 ** |
| LEL | 0.1572 | 0.0534 | 1.2735 | 0.1789 | 8.4556 | 0.0137 * |
| STL | 0.1178 | 0.0152 | 0.9970 | 0.2680 | 4.6324 | 0.0436 * |
| rproBCA1 | 0.1497 | 0.0067 | 0.3113 | 0.0456 | 4.9543 | 0.0384 * |
| rproBCA2 | 0.1322 | 0.0230 | 0.3115 | 0.0071 | 10.5430 | 0.0089 ** |
| rKAA1 | 0.1180 | 0.0106 | 0.2083 | 0.0173 | 6.2832 | 0.0244 * |
| rTachylectin-2 | 1.0590 | 0.0290 | 1.7830 | 0.0912 | 10.6975 | 0.0086 ** |
| DSA | 0.1032 | 0.0025 | 0.6463 | 0.0626 | 12.2616 | 0.0066 ** |
| PWM | 0.1213 | 0.0053 | 0.6655 | 0.1230 | 6.2499 | 0.0247 * |
| UDA | 0.1215 | 0.0078 | 0.7588 | 0.1920 | 4.6904 | 0.0426 * |
| WFL | 0.1580 | 0.0042 | 0.4330 | 0.0573 | 6.7716 | 0.0211 * |

As is evident from the results shown in Table 7, it was possible to distinguish between the species of the skin resident Staphylococcus, namely between Staphylococcus epidermidis at the stationary phase and Staphylococcus capitis at the stationary phase by using CBA, LEL, STL, proBCA1, proBCA2, KAA1, Tachylectin-2, DSA, PWM, UDA, or WFL.

### Example 5

### Selection of Lectins Capable of Distinguishing Between Staphylococcus aureus at Stationary Phase and Other Staphylococci at Stationary Phase

The above-described absorbance data was used to perform the significance test, and the lectins capable of distinguishing between Staphylococcus aureus at the stationary phase and other Staphylococci (skin resident Staphylococci: Staphylococcus epidermidis and Staphylococcus capitis) at the stationary phase were selected. The obtained results together with the conditions of the significance test are shown in Table 8.

TABLE 8
Analysis Conditions

| Type of Test: | T test | |
|---|---|---|
| S. aureus (Group 1) | S. aureus ATCC 6538 | S. aureus ATCC 27217 |
| S. epidermidis + S. capitis | S. epidermidis ATCC 12228 | S. epidermidis ATCC 14990 |
| (Group 2) | S. capitis ATCC 27840 | S. capitis ATCC 35661 |

| | |
|---|---|
| significance level: | 0.05 ns: no significant difference *: p < 0.05 **: p < 0.01 ***: p < 0.001 |

| | Group 1 | Group 1 | Group 2 | Group 2 | | |
|---|---|---|---|---|---|---|
| | Mean | Standard | Mean | Standard | | |
| Lectins | Value | Deviation | Value | Deviation | T value | P value |
| HAA | 0.9688 | 0.0449 | 0.5909 | 0.1292 | 3.8232 | 0.0187 * |
| HPA | 1.1643 | 0.0025 | 0.8035 | 0.1997 | 3.6122 | 0.0364 * |
| rBCL11 | 1.3123 | 0.1729 | 0.7819 | 0.2246 | 2.8769 | 0.0452 * |

As is evident from the results shown in Table 8, it was possible to distinguish between Staphylococcus aureus at the stationary phase and the other Staphylococci at the stationary phase by using HAA, HPA, or BCL11.

### Example 6

### Selection of Lectins Capable of Distinguishing Between Staphylococcus capitis at Stationary Phase and Other Staphylococci at Stationary Phase

The above-described absorbance data was used to perform the significance test, and the lectins capable of distinguishing between Staphylococcus capitis at the stationary phase and other Staphylococci (Staphylococcus aureus and Staphylococcus epidermidis) at the stationary phase were selected. The obtained results together with the conditions of the significance test are shown in Table 9.

TABLE 9
Analysis Conditions

| Type of Test: | T test | |
|---|---|---|
| S. aureus + S. epidermidis | S. aureus ATCC 6538 | S. aureus ATCC 27217 |
| (Group 1) | S. epidermidis ATCC 12228 | S. epidermidis ATCC 14990 |
| S. capitis (Group 2) | S. capitis ATCC 27840 | S. capitis ATCC 35661 |

| | |
|---|---|
| significance level: | 0.05 ns: no significant difference *: p < 0.05 **: p < 0.01 ***: p < 0.001 |

| | Group 1 | Group 1 | Group 2 | Group 2 | | |
|---|---|---|---|---|---|---|
| | Mean | Standard | Mean | Standard | | |
| Lectins | Value | Deviation | Value | Deviation | T value | P value |
| CBA | 0.1054 | 0.0084 | 0.1330 | 0.0035 | 4.2561 | 0.0131 * |
| LEL | 0.3736 | 0.2523 | 1.2735 | 0.1789 | 4.4015 | 0.0117 * |
| STL | 0.2240 | 0.1239 | 0.9970 | 0.2680 | 5.2004 | 0.0065 ** |
| rproBCA1 | 0.1619 | 0.0361 | 0.3113 | 0.0456 | 4.4558 | 0.0112 * |
| rproBCA2 | 0.1584 | 0.0467 | 0.3115 | 0.0071 | 4.3566 | 0.0121 * |
| rKAA1 | 0.1209 | 0.0153 | 0.2083 | 0.0173 | 6.3593 | 0.0031 ** |
| rTachylectin-2 | 1.2029 | 0.1682 | 1.7830 | 0.0912 | 4.3876 | 0.0118 * |
| rULL | 1.0627 | 0.1382 | 1.6790 | 0.2864 | 3.8125 | 0.0189 * |
| DSA | 0.1509 | 0.0814 | 0.6463 | 0.0626 | 7.4157 | 0.0018 ** |
| PWM | 0.1455 | 0.0406 | 0.6655 | 0.1230 | 8.4758 | 0.0011 ** |
| UDA | 0.1610 | 0.0582 | 0.7588 | 0.1920 | 6.3655 | 0.0031 ** |
| WFL | 0.1844 | 0.0686 | 0.4330 | 0.0573 | 4.3520 | 0.0121 * |

As is evident from the results shown in Table 9, it was possible to distinguish between Staphylococcus capitis at the stationary phase and the other Staphylococci at the stationary phase by using CBA, LEL, STL, proBCA1, proBCA2, KAA1, Tachylectin-2, ULL, DSA, PWM, UDA, or WFL.

### Example 7

### Selection of Lectins Capable of Distinguishing Between Staphylococcus epidermidis at Stationary Phase and Other Staphylococci at Stationary Phase

The above-described absorbance data was used to perform the significance test, and the lectins capable of distinguishing between Staphylococcus epidermidis at the stationary phase and other Staphylococci (Staphylococcus aureus and Staphylococcus capitis) at the stationary phase were selected. The obtained results together with the conditions of the significance test are shown in Table 10.

TABLE 10
Analysis Conditions

| Type of Test: | T test | |
|---|---|---|
| S. aureus + S. capitis | S. aureus ATCC 6538 | S. aureus ATCC 27217 |
| (Group 1) | S. capitis ATCC 27840 | S. capitis ATCC 35661 |
| S. epidermidis (Group 2) | S. epidermidis ATCC 12228 | S. epidermidis ATCC 14990 |

| | |
|---|---|
| significance level: | 0.05 ns: no significant difference *: p < 0.05 **: p < 0.01 ***: p < 0.001 |

| | Group 1 | Group 1 | Group 2 | Group 2 | | |
|---|---|---|---|---|---|---|
| | Mean | Standard | Mean | Standard | | |
| Lectin | Value | Deviation | Value | Deviation | T value | P value |
| rBCL11 | 1.1363 | 0.2306 | 0.6035 | 0.1358 | 2.9170 | 0.0434 * |

As is evident from the results shown in Table 10, it was possible to distinguish between Staphylococcus epidermidis at the stationary phase and the other Staphylococci at the stationary phase by using BCL11.

Based on the foregoing results, it became evident that the species at the stationary phase within the genus Staphylococcus could be distinguished using CBA, HAA, HPA, LEL, STL, proBCA1, proBCA2, KAA1, Tachylectin-2, ULL, DSA, PWM, UDA, WFL, hypninA3, or BCL11.

### Example 8

### Selection of Lectins Capable of Distinguishing Between Staphylococci at Stationary Phase and Bacteria at Stationary Phase Other Than Genus Staphylococcus

The above-described absorbance data was used to perform the significance test, and the lectins capable of distinguishing between Staphylococci at the stationary phase (Staphylococcus aureus, Staphylococcus epidermidis, and Staphylococcus capitis) and bacteria at the stationary phase other than the genus Staphylococcus (Escherichia coli, Bacillus subtilis, and Pseudomonas aeruginosa) were selected. The obtained results together with the conditions of the significance test are shown in Table 11.

TABLE 11
Analysis Conditions

| Type of Test: | T test | | |
|---|---|---|---|
| Others (Group 1) | E. coli ATCC 8739 | P. aeruginosa ATCC 9027 | B. subtilis ATCC 6633 |
| Staphylococci | S. aureus ATCC 6538 | S. aureus ATCC 27217 | |
| (Group 2) | S. epidermidis ATCC 12228 | S. epidermidis ATCC 14990 | |
| | S. capitis ATCC 27840 | S. capitis ATCC 35661 | |

| significance level: | 0.05 ns: no significant difference *: p < 0.05 **: p < 0.01 ***: p < 0.001 | | | | | |
|---|---|---|---|---|---|---|
| | Group 1 | Group 1 | Group 2 | Group 2 | | |
| | Mean | Standard | Mean | Standard | | |
| Lectins | Value | Deviation | Value | Deviation | T value | P value |
| GS-II | 0.2967 | 0.2092 | 1.1016 | 0.1732 | 6.1785 | 0.0005 *** |
| HAA | 0.1408 | 0.0205 | 0.7168 | 0.2202 | 6.3518 | 0.0014 ** |
| HPA | 0.1522 | 0.0196 | 0.9237 | 0.2421 | 7.7545 | 0.0006 *** |
| LEL | 0.1067 | 0.0058 | 0.6736 | 0.5104 | 2.7203 | 0.0418 * |
| PVL | 0.1413 | 0.0473 | 0.9099 | 0.2649 | 4.8239 | 0.0019 ** |
| STL | 0.1317 | 0.0383 | 0.4940 | 0.2874 | 3.0349 | 0.0289 * |
| WGA | 0.1515 | 0.0424 | 0.8438 | 0.3123 | 5.3320 | 0.0031 ** |
| rBCL11 | 0.2152 | 0.0780 | 0.9507 | 0.3571 | 3.4141 | 0.0112 * |
| rTachylectin-2 | 0.6255 | 0.5442 | 1.3963 | 0.3292 | 2.7078 | 0.0303 * |
| rULL | 0.5658 | 0.2540 | 1.2682 | 0.3594 | 2.9856 | 0.0204 * |

As is evident from the results shown in Table 11, it was possible to distinguish between the Staphylococci at the stationary phase and the bacteria at the stationary phase other than the genus Staphylococcus by using GS-II (GSL-II), HAA, HPA, LEL, PVL, STL, WGA, BCL11, Tachylectin-2, or ULL.

Thus, HAA, HPA, LEL, STL, Tachylectin-2, ULL, and BCL11 can distinguish not only between species at the stationary phase within the genus Staphylococcus but also between Staphylococci at the stationary phase and the bacteria at the stationary phase other than the genus Staphylococcus. Also, it is possible to distinguish not only between species at the stationary phase within the genus Staphylococcus but also between Staphylococci at the stationary phase and the bacteria at the stationary phase other than the genus Staphylococcus by combining the lectins according to the present invention capable of distinguishing between species within the genus Staphylococcus and a lectin among GS-II (GSL-II), PVL and WGA.

Note that the sources of the lectins capable of distinguishing the Staphylococci and the bacteria other than the genus Staphylococcus are described below.

GS-II/GSL-II: derived from Griffonia simplicifolia;
PVL: derived from weeping widow (Psathyrella velutina); and
WGA: derived from wheat (Triticum vulgare).

### Example 9

### Verification on Distinguishing Between Species within the Genus Staphylococcus by Tachylectin-2

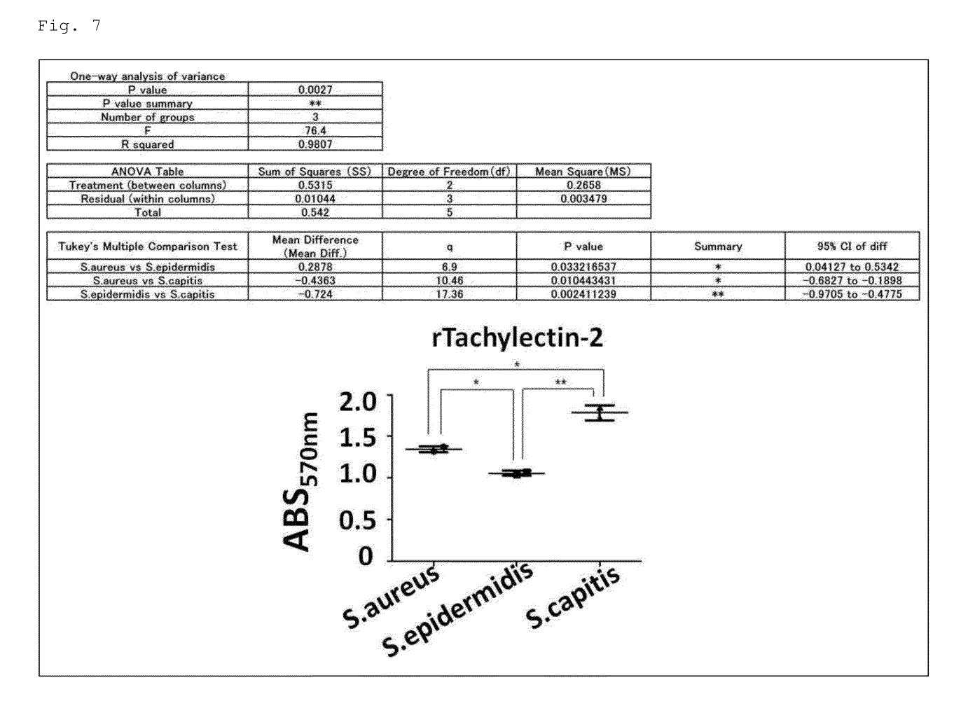

As described above, it became evident that Tachylectin-2 could distinguish even between any two species of Staphylococcus aureus, Staphylococcus epidermidis, and Staphylococcus capitis all at the stationary phase. Then, the usefulness of the present distinguishing method was attempted to be confirmed by the Tukey-Kramer multiple comparison method based on one-way analysis of variance. The obtained results are shown FIG. 7.

As is evident from the results shown in FIG. 7, it was possible to distinguish between any species of: Staphylococcus aureus and Staphylococcus epidermidis; Staphylococcus aureus and Staphylococcus capitis; and Staphylococcus epidermidis and Staphylococcus capitis, respectively, by using Tachylectin-2.

### Example 10

### Screening 2 of Lectins for Distinguishing Between Species within the Genus Staphylococcus at Stationary Phase

Different lectins from those described in Example 1 were targeted and the screening of the lectins that would bind to the bacteria at the stationary phase was carried out in the same manner as the method described in Example 1. Specifically, a microplate was immobilized with a total of 37 types of new lectins, including 5 types of commercially available lectins, 18 types of purified lectins from natural extracts, and 14 types of recombinant lectins, similarly to Example 1. Each strain described in Table 12 was shake-cultured in a Todd-Hewitt medium at 37° C. and 225 rpm. The stationary phase was taken as a state where culturing was conducted after the turbidity at 660 nm had reached 2.0 or more and the culture was sampled to prepare a bacterial suspension so that the turbidity at 660 nm became 1, similarly to Example 1.

Further, the plate immobilized with the 37 types of lectins was used to examine the binding of 9 types of bacteria described in Table 12 to the lectins by the plate centrifugation. Note that for each bacterium the measurement was independently conducted three times (N=3) per lectin. Also, there was used data obtained by subtracting a measured value of Blank whose well of the plate was not immobilized with any lectin from a measured value of absorbance per lectin; and the statistical analysis was performed. The statistical differences were verified by Welch's t-test (two-sided test) and the “P<0.05” was determined to be statically significant.

TABLE 12

| | Bacteria | ATCC No. | Type |
|---|---|---|---|
| 1 | Staphylococcus aureus subsp. | 6538 | food poisoning |
| | aureus | | bacterium |
| 2 | Staphylococcus aureus subsp. | 27217 | food poisoning |
| | aureus | | bacterium |
| 3 | Staphylococcus aureus subsp. | 25923 | food poisoning |
| | aureus | | bacterium |
| 4 | Staphylococcus epidermidis | 12228 | resident bacterium |
| 5 | Staphylococcus epidermidis | 14990 | resident bacterium |
| 6 | Staphylococcus epidermidis | 35547 | resident bacterium |
| 7 | Staphylococcus capitis subsp. | 27840 | resident bacterium |
| | capitis | | |
| 8 | Staphylococcus capitis subsp. | 35661 | resident bacterium |
| | capitis | | |
| 9 | Staphylococcus capitis subsp. | 27843 | resident bacterium |
| | capitis | | |

### Example 11

### Selection 2 of Lectins Capable of Distinguishing Between Staphylococcus aureus at Stationary Phase and Staphylococcus capitis at Stationary Phase

The above-described absorbance data was used to perform the significance test, and the lectins capable of distinguishing between Staphylococcus aureus at the stationary phase and other Staphylococcus at the stationary phase (a skin resident Staphylococcus; Staphylococcus capitis) were selected. The obtained results together with the conditions of the significance test are shown in Table 13.

TABLE 13
S. aureus VS. S. capitis (Welch)

| Type of Test: | t test | |
|---|---|---|
| Group 1: | Staphylococcus aureus | Staphylococcus aureus sub sp. aureus #27217 |
| | sub sp. aureus #6538 | |
| | Staphylococcus aureus | |
| | sub sp. aureus #25923 | |
| Group 2: | Staphylococcus capitis | Staphylococcus capitis sub sp. capitis #35661 |
| | sub sp. capitis #27840 | |
| | Staphylococcus capitis | |
| | sub sp. capitis #27843 | |

| significance level: | 0.05 *: p < 0.05 **: p < 0.01 ***: p < 0.001 | | | | | |
|---|---|---|---|---|---|---|
| | Group 1 | | Group 2 | | | |
| | Mean | Standard | Mean | Standard | | |
| Lectins | Value | Deviation | Value | Deviation | T value | P value |
| rRSL | 0.261 | 0.219 | 1.088 | 0.180 | −5.058 | 7.9E−03 ** |
| rCHA-1 | 0.083 | 0.053 | 0.302 | 0.061 | −4.702 | 9.7E−03 ** |
| rCLA | 0.489 | 0.351 | 1.695 | 0.339 | −4.286 | 0.013 * |
| rTachylectin-3 | 0.408 | 0.213 | 1.297 | 0.304 | −4.149 | 0.018 * |
| BanLec | 0.611 | 0.137 | 0.048 | 0.015 | 7.048 | 0.018 * |
| rAPI 144 | 0.524 | 0.421 | 1.461 | 0.346 | −2.977 | 0.043 * |
| AC-avranin | 0.365 | 0.484 | 1.444 | 0.215 | −3.530 | 0.044 * |
| rBML11b | 1.066 | 0.283 | 2.215 | 0.540 | −3.264 | 0.047 * |

As is evident from the results shown in Table 13, it was possible to distinguish between Staphylococcus aureus at the stationary phase and the other Staphylococcus at the stationary phase (Staphylococcus capitis) by using RSL, CHA-1, CLA, Tachylectin-3, BanLec, API 144, AC-avranin, or BML11b.

Therefore, when combined with the results of Example 3, it became evident that Staphylococcus aureus at the stationary phase and Staphylococcus capitis at the stationary phase could be distinguished by HAA, LEL, Tachylectin-2, DSA, PWM, hypninA3, RSL, CHA-1, CLA, Tachylectin-3, BanLec, API 144, AC-avranin, or BML11b.

### Example 12

### Selection 2 of Lectins Capable of Distinguishing Between Staphylococcus epidermidis at Stationary Phase and Staphylococcus capitis at Stationary Phase

The above-described absorbance data was used to perform the significance test, and the lectins capable of distinguishing between species of skin resident Staphylococci at the stationary phase, specifically between Staphylococcus epidermidis at the stationary phase and Staphylococcus capitis at the stationary phase were selected. The obtained results together with the conditions of the significance test are shown in Table 14.

TABLE 14
S. epidermidis VS. S. capitis (Welch)

| Type of Test: | t test | |
|---|---|---|
| Group 1: | Staphylococcus epidermidis | Staphylococcus epidermidis #14990 |
| | #12228 | |
| | Staphylococcus epidermidis | |
| | #35547 | |
| Group 2: | Staphylococcus capitis | Staphylococcus capitis sub sp. capitis #35661 |
| | sub sp. capitis #27840 | |
| | Staphylococcus capitis | |
| | sub sp. capitis #27843 | |

| significance level: | 0.05 *: p < 0.05 **: p < 0.01 ***: p < 0.001 | | | | | |
|---|---|---|---|---|---|---|
| | Group 1 | | Group 2 | | | |
| | Mean | Standard | Mean | Standard | | |
| Lectins | Value | Deviation | Value | Deviation | T value | P value |
| rFVE | 0.048 | 0.031 | 0.279 | 0.011 | −12.115 | 3.0E−03 ** |
| Pro-CFA II | 0.056 | 0.020 | 0.968 | 0.154 | −10.203 | 8.5E−03 ** |
| rRSL | 0.045 | 0.022 | 1.088 | 0.180 | −9.978 | 9.0E−03 ** |
| rCHA-1 | 0.023 | 0.004 | 0.302 | 0.061 | −7.929 | 0.015 * |
| rBML11b | 0.628 | 0.459 | 2.215 | 0.540 | −3.876 | 0.019 * |
| rTachylectin-3 | 0.071 | 0.034 | 1.297 | 0.304 | −6.940 | 0.019 * |
| Pro-CFA I | 0.062 | 0.031 | 1.084 | 0.401 | −4.405 | 0.047 * |

As is evident from the results shown in Table 14, it was possible to distinguish between the species of skin resident Staphylococci at the stationary phase, specifically between Staphylococcus epidermidis at the stationary phase and Staphylococcus capitis at the stationary phase by using FVE, Pro-CFA II, RSL, CHA-1, BML11b, Tachylectin-3, or Pro-CFA I.

Therefore, when combined with the results of Example 4, it became evident that Staphylococcus epidermidis at the stationary phase and Staphylococcus capitis at the stationary phase could be distinguished by CBA, LEL, STL, proBCA1, proBCA2, KAA1, Tachylectin-2, DSA, PWM, UDA, WFL, FVE, Pro-CFA II, RSL, CHA-1, BML11b, Tachylectin-3, or Pro-CFA I.

### Example 13

### Selection of Lectins Capable of Distinguishing Between Staphylococcus epidermidis at Stationary Phase and Other Staphylococci at Stationary Phase

The above-described absorbance data was used to perform the significance test, and the lectins capable of distinguishing between Staphylococcus epidermidis at the stationary phase and other Staphylococci at the stationary phase (Staphylococcus aureus and Staphylococcus capitis) were selected. The obtained results together with the conditions of the significance test are shown in Table 15.

TABLE 15
S. epidermidis VS. S. aureus & S. capitis (Welch)

| Type of Test: | t test | |
|---|---|---|
| Group 1: | Staphylococcus epidermidis | Staphylococcus epidermidis #14990 |
| | #12228 | |
| | Staphylococcus epidermidis | |
| | #35547 | |
| Group 2: | Staphylococcus aureus sub | Staphylococcus aureus sub sp. aureus #27217 |
| | sp. aureus #6538 | |
| | Staphylococcus aureus sub | |
| | sp. aureus #25923 | |
| | Staphylococcus capitis sub | Staphylococcus capitis sub sp. capitis #35661 |
| | sp. capitis #27840 | |
| | Staphylococcus capitis sub | |
| | sp. capitis #27843 | |

| significance level: | 0.05 *: p < 0.05 **: p < 0.01 ***: p < 0.001 | | | | | |
|---|---|---|---|---|---|---|
| | Group 1 | | Group 2 | | | |
| | Mean | Standard | Mean | Standard | | |
| Lectins | Value | Deviation | Value | Deviation | T value | P value |
| Pro-CFA II | 0.056 | 0.020 | 0.737 | 0.323 | −5.139 | 3.5E−03 ** |
| aIgCSA | 1.362 | 0.165 | 0.686 | 0.380 | 3.714 | 7.5E−03 ** |
| Pro-CFA I | 0.062 | 0.031 | 0.855 | 0.455 | −4.253 | 7.7E−03 ** |
| rFVE | 0.048 | 0.031 | 0.206 | 0.108 | −3.304 | 0.015 * |
| rTachylectin-3 | 0.071 | 0.034 | 0.852 | 0.541 | −3.525 | 0.016 * |
| rCLA | 0.073 | 0.079 | 1.092 | 0.729 | −3.383 | 0.018 * |
| rAPI 144 | 0.143 | 0.182 | 0.993 | 0.618 | −3.109 | 0.019 * |
| rMPA1 | 0.000 | 0.000 | 0.169 | 0.125 | −3.310 | 0.021 * |
| rCHA-1 | 0.023 | 0.004 | 0.192 | 0.130 | −3.183 | 0.024 * |
| rRSL | 0.045 | 0.022 | 0.674 | 0.487 | −3.156 | 0.025 * |
| rUPL-1 | 0.044 | 0.017 | 0.891 | 0.689 | −3.008 | 0.030 * |
| AC-avranin | 0.075 | 0.094 | 0.904 | 0.680 | −2.934 | 0.030 * |
| rBML11c | 0.225 | 0.168 | 1.073 | 0.736 | −2.683 | 0.037 * |
| rBML11b | 0.628 | 0.459 | 1.641 | 0.738 | −2.522 | 0.043 * |

As is evident from the results shown in Table 15, it was possible to distinguish between Staphylococcus epidermidis at the stationary phase and the other Staphylococci at the stationary phase by using Pro-CFA II, algCSA, Pro-CFA I, FVE, Tachylectin-3, CLA, API 144, MPA1, CHA-1, RSL, UPL-1, AC-avranin, BML11c, or BML11b.

Therefore, when combined with the results of Example 7, it became evident that Staphylococcus epidermidis at the stationary phase and the other Staphylococci at the stationary phase could be distinguished by BCL11, Pro-CFA II, algCSA, Pro-CFA I, FVE, Tachylectin-3, CLA, API 144, MPA1, CHA-1, RSL, UPL-1, AC-avranin, BML11c, or BML11b.

### Example 14

### Selection 2 of Lectins Capable of Distinguishing Between Staphylococcus capitis at Stationary Phase and Other Staphylococci at Stationary Phase

The above-described absorbance data was used to perform the significance test, and the lectins capable of distinguishing between Staphylococcus capitis at the stationary phase and other Staphylococci at the stationary phase (Staphylococcus aureus and Staphylococcus epidermidis) were selected. The obtained results together with the conditions of the significance test are shown in Table 16.

TABLE 16
S. aureus & S. epidermidis VS. S. capitis (Welch)

| Type of Test: | t test | |
|---|---|---|
| Group 1: | Staphylococcus aureus sub | Staphylococcus aureus sub sp. aureus #27217 |
| | sp. aureus #6538 | |
| | Staphylococcus aureus sub | |
| | sp. aureus #25923 | |
| | Staphylococcus epidermidis | Staphylococcus epidermidis #14990 |
| | #12228 | |
| | Staphylococcus epidermidis | |
| | #35547 | |
| Group 2: | Staphylococcus capitis sub | Staphylococcus capitis sub sp. capitis #35661 |
| | sp. capitis #27840 | |
| | Staphylococcus capitis sub | |
| | sp. capitis #27843 | |

| significance level: | 0.05 *: p < 0.05 **: p < 0.01 ***: p < 0.001 | | | | | |
|---|---|---|---|---|---|---|
| | Group 1 | | Group 2 | | | |
| | Mean | Standard | Mean | Standard | | |
| Lectins | Value | Deviation | Value | Deviation | T value | P value |
| AC-avranin | 0.220 | 0.350 | 1.444 | 0.215 | −6.471 | 5.1E−04 *** |
| BanLec | 0.649 | 0.232 | 0.048 | 0.015 | 6.308 | 1.4E−03 ** |
| rRSL | 0.153 | 0.182 | 1.088 | 0.180 | −7.321 | 1.6E−03 ** |
| Pro-CFA II | 0.281 | 0.304 | 0.968 | 0.154 | −4.504 | 2.9E−03 ** |
| rFVE | 0.091 | 0.089 | 0.279 | 0.011 | −5.098 | 3.2E−03 ** |
| rCLA | 0.281 | 0.322 | 1.695 | 0.339 | −6.005 | 4.2E−03 ** |
| rCHA-1 | 0.053 | 0.047 | 0.302 | 0.061 | −6.221 | 6.5E−03 ** |
| rAPI 144 | 0.333 | 0.357 | 1.461 | 0.346 | −4.558 | 9.1E−03 ** |
| rTachylectin-3 | 0.240 | 0.229 | 1.297 | 0.304 | −5.316 | 0.011 * |
| rBML11b | 0.847 | 0.417 | 2.215 | 0.540 | −3.850 | 0.027 * |
| rBCL11d | 0.938 | 0.286 | 1.786 | 0.359 | −3.565 | 0.032 * |

As is evident from the results shown in Table 16, it was possible to distinguish between Staphylococcus capitis at the stationary phase and the other Staphylococci at the stationary phase by using AC-avranin, BanLec, RSL, Pro-CFA II, FVE, CLA, CHA-1, API 144, Tachylectin-3, BML11b, or BML11d.

Therefore, when combined with the results of Example 6, it became evident that Staphylococcus capitis at the stationary phase and the other Staphylococci at the stationary phase could be distinguished by CBA, LEL, STL, proBCA1, proBCA2, KAA1, Tachylectin-2, ULL, DSA, PWM, UDA, WFL, AC-avranin, BanLec, RSL, Pro-CFA II, FVE, CLA, CHA-1, API 144, Tachylectin-3, BML11b, or BCL11d.

### Example 15

### Screening of Lectins for Distinguishing Between Species within the Genus Staphylococcus at Logarithmic Growth Phase

<Lectins Used>

Screening of lectins capable of binding to bacteria at the logarithmic growth phase was carried out using a total of 153 types of lectins, including 92 types of commercially available lectins, 25 types of purified lectins from natural extracts, and 36 types of recombinant lectins.

Note that the commercially available lectins were purchased from EY Laboratories, Vector Laboratories, Seikagaku Corporation, Sigma-Aldrich Corporation and Wako Pure Chemical Industries, Ltd. The purified lectins from natural extracts were prepared at Hiroshima University or GLYENCE Co., Ltd; and the recombinant lectins were constructed at GLYENCE Co. Ltd.

<Antiserum Used>

S. epidermidis ATCC14990 was irradiated with ultraviolet ray and the bacterial cells were confirmed not to grow. Rabbit was immunized with the bacterial cell as an antigen; and an antiserum that would bind to Staphylococcus was prepared and provided for use in the present example.

<Strains Used>

The strains which were used in the screening experiment are shown in Table 17. There were used five strains of Staphylococcus aureus as food poisoning bacteria, each three strains of Staphylococcus epidermidis and Staphylococcus capitis as resident bacteria, each one strain of S. haemolyticus and S. homominis as another Staphylococcus, and each one strain of Escherichia coli, Bacillus subtilis, and Pseudomonas aeruginosa as a bacterium other than Staphylococci. The respective strains were purchased from The American Type Culture Collection (ATCC).

TABLE 17

| | Bacteria | ATCC No. | Type |
|---|---|---|---|
| 1 | Staphylococcus aureus subsp. | 6538 | food poisoning |
| | aureus | | bacterium |
| 2 | Staphylococcus aureus subsp. | 27217 | food poisoning |
| | aureus | | bacterium |
| 3 | Staphylococcus aureus subsp. | 10832 | food poisoning |
| | aureus | | bacterium |
| 4 | Staphylococcus aureus subsp. | 25923 | food poisoning |
| | aureus | | bacterium |
| 5 | Staphylococcus aureus subsp. | 27734 | food poisoning |
| | aureus | | bacterium |
| 6 | Staphylococcus epidermidis | 12228 | resident bacterium |
| 7 | Staphylococcus epidermidis | 14990 | resident bacterium |
| 8 | Staphylococcus epidermidis | 35547 | resident bacterium |
| 9 | Staphylococcus capitis subsp. | 27840 | resident bacterium |
| | capitis | | |
| 10 | Staphylococcus capitis subsp. | 35661 | resident bacterium |
| | capitis | | |
| 11 | Staphylococcus capitis subsp. | 27843 | resident bacterium |
| | capitis | | |
| 12 | Staphylococcus haemolyticus | 29970 | resident bacterium |
| 13 | Staphylococcus hominis subsp. | 27844 | resident bacterium |
| | hominis | | |
| 14 | Escherichia coli | 8739 | reference bacterium |
| 15 | Bacillus subtilis subsp. spizizenii | 6623 | reference bacterium |
| 16 | Psedomonas aeruginosa | 9027 | reference bacterium |

<Lectin Screening by Plate Centrifugation>

Each 100 μl of the lectins or the antiserum that had been diluted to 10 μg/ml with a carbonate buffer (pH 9.6) was sensitized overnight at 4° C. on a microplate (manufactured by Nunc A/S; surface-treatment maxisorp; Catalog No. 445101), and the lectins were immobilized on the microplate. Next, the lectin solution or the like was removed, to which 300 μl of a blocking reagent for immunoassay N101 (manufactured by NOF Corporation) that had been diluted five times was then added. Blocking was conducted at room temperature for 3 h.

Each strain shown in Table 17 was further shake-cultured in a Todd-Hewitt medium at 37° C. and 225 rpm, and the turbidity at 660 nm was measured over time to depict a growth curve per strain. Based on the growth curve the bacterium at the state where its turbidity is from 0.6 to 1.0 was sampled as a bacterium at the late phase of logarithmic growth. Next, after the sampled culture was centrifuged to recover bacterial cells, it was washed with PBS three times and a bacterial suspension was prepared with 1% BSA/CM-TBS (TBS, 1% BSA, 1 mM CaCl₂, and 1 mm MnCl₂) so as to provide a turbidity of 1 at a wavelength of 660 nm. Further, 100 μl of the bacterial suspension was dispensed on the plate and centrifugation was carried out with a centrifuge at 4° C. and 510×g for 10 min. After centrifugation, 250 μl of CM-TBS (TBS, 1% BSA, 1 mM CaCl₂, and 1 mm MnCl₂) was gently added to the plate, which was sealed with a plate seal (manufactured by Nunc A/C; Catalog No. 236366). The plate was then inverted and centrifuged with a centrifuge at 4° C. and 160×g for 5 min. After centrifugation, 250 μl of a supernatant was removed from the plate, to which 100 μl of a TBS-CM/0.5% glutaraldehyde solution was added and fixed at room temperature for 1 h. After the glutaraldehyde solution was removed, the plate was washed with PBS and there was added 100 μl of a 2.3% crystal violet solution. Staining was carried out at room temperature for 1 h and excess dye was washed with running water. Thereafter, there was added 100 μl of 99.5% ethanol and the dye was eluted at room temperature for 1 h. Absorbance at 570 nm was quantified with a plate reader.

Further, the plate immobilized with the 153 types of the above-described lectins was used to examine the binding of 16 types of the bacteria described in Table 17 to the lectins by the plate centrifugation. Note that for each bacterium the measurement was independently conducted three times (N=3) per lectin. Also, there was used data obtained by subtracting a measured value of Blank whose well of the plate was not immobilized with any lectin from a measured value of absorbance per lectin; and the statistical analysis was performed. The statistical differences were verified by Welch's t-test (two-sided test) and the “P<0.05” was determined to be statically significant.

### Example 16

### Selection of Lectins Capable of Distinguishing Between Staphylococcus aureus at Logarithmic Growth Phase and Other Staphylococci at Logarithmic Growth Phase

The above-described absorbance data was used to perform the significance test, and the lectins capable of distinguishing between Staphylococcus aureus at the logarithmic growth phase and other Staphylococci (Staphylococcus epidermidis, Staphylococcus capitis, Staphylococcus haemolyticus, and Staphylococcus hominis) at the logarithmic growth phase were selected. The obtained results together with the conditions of the significance test are shown in Table 18.

TABLE 18
S. aureus VS. Genus Staphylococcus Other Than Staphylococcus aureus (Welch)

| Type of Test: | t test (Welch) | |
|---|---|---|
| Group 1: | Staphylococcus aureus subsp. | Staphylococcus aureus subsp. aureus #27217 |
| | aureus #6538 | |
| | Staphylococcus aureus subsp. | Staphylococcus aureus subsp. aureus #35923 |
| | aureus #10832 | |
| | Staphylococcus aureus subsp. | |
| | aureus #27734 | |
| Group 2: | Staphylococcus epidermidis | Staphylococcus epidermidis #14990 |
| | #12228 | |
| | Staphylococcus epidermidis | |
| | #35547 | |
| | Staphylococcus capitis subsp. | Staphylococcus capitis subsp. capitis #35661 |
| | capitis #27840 | |
| | Staphylococcus capitis subsp. | |
| | capitis #27843 | |
| | Staphylococcus haemolyticus | Staphylococcus hominis subsp. hominis #27844 |
| | #29970 | |

| significance level: | 0.05 *: p < 0.05 **: p < 0.01 ***: p < 0.001 | | | | | |
|---|---|---|---|---|---|---|
| | Group 1 | | Group 2 | | | |
| | Mean | Standard | Mean | Standard | | |
| Lectins | Value | Deviation | Value | Deviation | T value | P value |
| rCV-n | 0.330 | 0.087 | 0.105 | 0.106 | 4.176 | 1.9E−03 ** |
| AC-avranin | 0.314 | 0.104 | 0.098 | 0.061 | 4.224 | 6.0E−03 ** |
| PMA | 0.129 | 0.044 | 0.041 | 0.036 | 3.737 | 6.9E−03 ** |
| GSL-II | 1.015 | 0.205 | 1.464 | 0.306 | −3.178 | 8.9E−03 ** |
| rOAA | 0.401 | 0.178 | 0.051 | 0.064 | 4.239 | 9.6E−03 ** |
| rACG | 0.128 | 0.053 | 0.031 | 0.009 | 4.026 | 0.015 * |
| rApl 144 | 0.481 | 0.202 | 0.153 | 0.128 | 3.253 | 0.017 * |
| PNA | 0.576 | 0.291 | 0.109 | 0.135 | 3.367 | 0.019 * |
| algMPL | 0.368 | 0.216 | 0.017 | 0.017 | 3.624 | 0.022 * |
| Pro-CFA II | 0.612 | 0.233 | 0.266 | 0.242 | 2.570 | 0.030 * |
| CEA | 0.223 | 0.152 | 0.016 | 0.020 | 3.010 | 0.038 * |
| rGarlic lectin | 0.795 | 0.298 | 0.384 | 0.300 | 2.416 | 0.040 * |
| TL | 0.219 | 0.113 | 0.074 | 0.074 | 2.552 | 0.042 * |
| MOA | 0.429 | 0.221 | 0.151 | 0.132 | 2.545 | 0.045 * |
| rMPA2 | 0.373 | 0.232 | 0.088 | 0.096 | 2.619 | 0.048 * |
| DSA | 0.056 | 0.034 | 0.281 | 0.268 | −2.350 | 0.049 * |

As is evident from the results shown in Table 18, it was possible to distinguish between Staphylococcus aureus at the logarithmic growth phase and the other Staphylococci at the logarithmic growth phase by using CV-N, AC-avranin, PMA, GSL-II, OAA, ACG, API 144, PNA, algMPL, Pro-CFA II, CBA, Garlic lectin, TL, MOA, MPA2, or DSA.

### Example 17

### Selection of Lectins Capable of Distinguishing Between Staphylococcus epidermidis at Logarithmic Growth Phase and Other Staphylococci at Logarithmic Growth Phase

The above-described absorbance data was used to perform the significance test, and the lectins capable of distinguishing between Staphylococcus epidermidis at the logarithmic growth phase and other Staphylococci (Staphylococcus aureus, Staphylococcus capitis, Staphylococcus haemolyticus, and Staphylococcus hominis) at the logarithmic growth phase were selected. The obtained results together with the conditions of the significance test are shown in Table 19.

TABLE 19
S. epidermidis VS. Genus Staphylococcus Other Than Staphylococcus epidermidis (Welch)

| Type of Test: | t test (Welch) | |
|---|---|---|
| Group 1: | Staphylococcus epidermidis | Staphylococcus epidermidis #14990 |
| | #12228 | |
| | Staphylococcus epidermidis | |
| | #35547 | |
| Group 2: | Staphylococcus aureus subsp. | Staphylococcus aureus subsp. aureus #27217 |
| | aureus #6538 | |
| | Staphylococcus aureus subsp. | Staphylococcus aureus subsp. aureus #25923 |
| | aureus #10832 | |
| | Staphylococcus aureus subsp. | |
| | aureus #27734 | |
| | Staphylococcus capitis subsp. | Staphylococcus capitis subsp. capitis #35661 |
| | capitis #27840 | |
| | Staphylococcus capitis subsp. | |
| | capitis #27843 | |
| | Staphylococcus haemolyticus | Staphylococcus hominis subsp. hominis #27844 |
| | #29970 | |

| significance level: | 0.05 *: p < 0.05 **: p < 0.01 ***: p < 0.001 | | | | | |
|---|---|---|---|---|---|---|
| | Group 1 | | Group 2 | | | |
| | Mean | Standard | Mean | Standard | | |
| Lectins | Value | Deviation | Value | Deviation | T value | P value |
| SHA | 0.821 | 0.018 | 0.146 | 0.078 | −4.661 | 7.0E−04 *** |
| rRSL | 0.098 | 0.046 | 0.327 | 0.160 | −4.003 | 2.1E−03 ** |
| rproBCA2 | 0.143 | 0.106 | 0.559 | 0.253 | −3.835 | 3.4E−03 ** |
| rproBCA1 | 0.139 | 0.085 | 0.528 | 0.356 | −3.166 | 9.0E−03 ** |
| CPA | 0.113 | 0.073 | 0.472 | 0.342 | −3.091 | 0.010 * |
| UEA-II | 0.068 | 0.033 | 0.303 | 0.231 | −3.112 | 0.011 * |
| LAA | 0.175 | 0.104 | 0.476 | 0.228 | −3.224 | 0.012 * |
| rCHA-1 | 0.174 | 0.106 | 0.539 | 0.343 | -2.937 | 0.014 * |
| rOAA | 0.027 | 0.012 | 0.223 | 0.219 | −2.920 | 0.016 * |
| LPA | 0.121 | 0.126 | 0.476 | 0.391 | −2.475 | 0.032 * |
| algMPL | 0.016 | 0.018 | 0.193 | 0.235 | −2.355 | 0.042 * |

As is evident from the results shown in Table 19, it was possible to distinguish between Staphylococcus epidermidis at the logarithmic growth phase and the other Staphylococci at the logarithmic growth phase by using SHA, RSL, ProBCA2, ProBCA1, CPA, UEA-II, LAA, CHA-1, OAA, LPA, or algMPL.

### Example 18

### Selection of Lectins Capable of Distinguishing Between Staphylococcus capitis at Logarithmic Growth Phase and Other Staphylococci at Logarithmic Growth Phase

The above-described absorbance data was used to perform the significance test, and the lectins capable of distinguishing between Staphylococcus capitis at the logarithmic growth phase and other Staphylococci at the logarithmic growth phase (Staphylococcus aureus, Staphylococcus epidermidis, Staphylococcus haemolyticus, and Staphylococcus hominis) were selected. The obtained results together with the conditions of the significance test are shown in Table 20.

TABLE 20
S. capitis VS Genus Staphylococcus Other Than Staphylococcus capitis (Welch)

| Type of Test: | t test (Welch) | |
|---|---|---|
| Group 1: | Staphylococcus capitis subsp. | Staphylococcus capitis subsp. capitis #35661 |
| | capitis #27840 | |
| | Staphylococcus capitis subsp. | |
| | capitis #27843 | |
| Group 2: | Staphylococcus aureus subsp. | Staphylococcus aureus subsp. aureus #27217 |
| | aureus #6538 | |
| | Staphylococcus aureus sbusp. | |
| | aureus #10832 | |
| | Staphylococcus aureus subsp. | |
| | aureus #27734 | |
| | Staphylococcus epidermidis | Staphylococcus epidermidis #14990 |
| | #12228 | |
| | Staphylococcus epidermidis | |
| | #35547 | |
| | Staphylococcus haemolyticus | Staphylococcus hominis subsp. hominis #27844 |
| | #29970 | |

| significance level: | 0.05 *: p < 0.05 **: p < 0.01 ***: p < 0.001 | | | | | |
|---|---|---|---|---|---|---|
| | Group 1 | | Group 2 | | | |
| | Mean | Standard | Mean | Standard | | |
| Lectins | Value | Deviation | Value | Deviation | T value | P value |
| rpcoBCA2 | 0.921 | 0.086 | 0.325 | 0.185 | 7.769 | 5.6E−05 *** |
| LAA | 0.665 | 0.074 | 0.331 | 0.222 | 4.659 | 9.9E−04 *** |
| LPA | 0.791 | 0.039 | 0.275 | 0.347 | 4.607 | 1.1E−03 ** |
| rOAA | 0.008 | 0.007 | 0.239 | 0.213 | −3.414 | 7.6E−03 ** |
| rproBCA1 | 0.985 | 0.182 | 0.274 | 0.173 | 6.001 | 7.9E−03 ** |
| UEA-II | 0.595 | 0.128 | 0.145 | 0.108 | 5.530 | 0.013 * |
| CPA | 0.859 | 0.200 | 0.248 | 0.216 | 4.552 | 0.014 * |
| rCHA-1 | 0.930 | 0.222 | 0.312 | 0.213 | 4.258 | 0.021 * |
| PAA | 0.402 | 0.122 | 0.031 | 0.020 | 6.402 | 0.023 * |
| rPSL | 0.517 | 0.119 | 0.201 | 0.103 | 4.153 | 0.026 * |
| algMPL | 0.009 | 0.003 | 0.195 | 0.293 | −2.526 | 0.032 * |
| SHA | 0.208 | 0.056 | 0.090 | 0.077 | 2.940 | 0.035 * |

As is evident from the results shown in Table 20, it was possible to distinguish between Staphylococcus capitis at the logarithmic growth phase and the other Staphylococci at the logarithmic growth phase by using proBCA2, LAA, LPA, OAA, proBCA1, UEA-II, CPA, CHA-1, PAA, RSL, algMPL, or SHA.

Therefore, based on the results of Examples 17 and 18, it became evident that SHA, RSL, proBCA2, proBCA1, CPA, UEA-II, LAA, CHA-1, OAA, LPA, or algMPL could distinguish between Staphylococcus epidermidis at the logarithmic growth phase and the other Staphylococci at the logarithmic growth phase as well as between Staphylococcus capitis at the logarithmic growth phase and the other Staphylococci at the logarithmic growth phase.

### Example 19

### Selection of Lectins Capable of Distinguishing Between Staphylococcus aureus at Logarithmic Growth Phase and Staphylococcus epidermidis at Logarithmic Growth Phase

The above-described absorbance data was used to perform the significance test, and the lectins capable of distinguishing between Staphylococcus aureus at the logarithmic growth phase and Staphylococcus epidermidis at the logarithmic growth phase were selected. The obtained results together with the conditions of the significance test are shown in Table 21.

TABLE 21
S. aureus VS. S. epidermidis (Welch)

| Type of Test: | t test | |
|---|---|---|
| Group 1: | Staphylococcus aureus subsp. | Staphylococcus aureus subsp. aureus #27217 |
| | aureus #6538 | |
| | Staphylococcus aureus subsp. | Staphylococcus aureus subsp. aureus #25923 |
| | aureus #10832 | |
| | Staphylococcus aureus subsp. | |
| | aureus #27734 | |
| Group 2: | Staphylococcus epidermidis | Staphylococcus epidermidis #14990 |
| | #12228 | |
| | Staphylococus epidermis | |
| | #35547 | |

| significance level: | 0.05 *: p < 0.05 **: p < 0.01 ***: p < 0.001 | | | | | |
|---|---|---|---|---|---|---|
| | Group 1 | | Group 2 | | | |
| | Mean | Standard | Mean | Standard | | |
| Lectins | Value | Deviation | Value | Deviation | T value | P value |
| rCV-N | 0.330 | 0.087 | 0.040 | 0.020 | 7.168 | 1.1E−03 ** |
| SHA | 0.151 | 0.056 | 0.021 | 0.018 | 4.782 | 4.5E−03 ** |
| rOAA | 0.401 | 0.176 | 0.027 | 0.012 | 4.694 | 9.0E−03 ** |
| AC-avranin | 0.314 | 0.104 | 0.096 | 0.064 | 3.677 | 0.011 * |
| rproBCA2 | 0.464 | 0.130 | 0.143 | 0.106 | 3.807 | 0.012 * |
| UEA-II | 0.228 | 0.088 | 0.068 | 0.033 | 3.688 | 0.013 * |
| rACG | 0.128 | 0.053 | 0.029 | 0.009 | 4.067 | 0.013 * |
| PNA | 0.576 | 0.291 | 0.039 | 0.046 | 4.047 | 0.013 * |
| LAA | 0.502 | 0.173 | 0.175 | 0.104 | 3.343 | 0.016 * |
| TL | 0.219 | 0.113 | 0.028 | 0.016 | 3.719 | 0.018 * |
| MOA | 0.429 | 0.221 | 0.059 | 0.034 | 3.674 | 0.019 * |
| rRSL | 0.250 | 0.090 | 0.098 | 0.046 | 3.145 | 0.020 * |
| algMPL | 0.368 | 0.216 | 0.016 | 0.018 | 3.618 | 0.022 * |
| rproBCA1 | 0.397 | 0.153 | 0.139 | 0.085 | 3.059 | 0.022 * |
| rCHA-1 | 0.473 | 0.175 | 0.174 | 0.106 | 3.022 | 0.024 * |
| rMPA2 | 0.373 | 0.232 | 0.032 | 0.041 | 3.208 | 0.028 * |
| CBA | 0.223 | 0.152 | 0.002 | 0.004 | 3.232 | 0.032 * |
| CPA | 0.396 | 0.216 | 0.113 | 0.073 | 2.693 | 0.041 * |

As is evident from the results shown in Table 21, it was possible to distinguish between Staphylococcus aureus at the logarithmic growth phase and Staphylococcus epidermidis at the logarithmic growth phase by using CV-N, SHA, OAA, AC-avranin, proBCA2, UEA-II, ACG, PNA, LAA, TL, MOA, RSL, algMPL, proBCA1, CHA-1, MPA2, CBA, or CPA.

### Example 20

### Selection of Lectins Capable of Distinguishing Between Staphylococcus aureus at Logarithmic Growth Phase and Staphylococcus capitis at Logarithmic Growth Phase

The above-described absorbance data was used to perform the significance test, and the lectins capable of distinguishing between Staphylococcus aureus at the logarithmic growth phase and Staphylococcus capitis at the logarithmic growth phase were selected. The obtained results together with the conditions of the significance test are shown in Table 22.

TABLE 22
S. aureus VS. S. capitis (Welch)

| Type of Test: | t test | |
|---|---|---|
| Group 1: | Staphylococcus aureus sub | Staphylococcus aureus sub sp. aureus #27217 |
| | sp. aureus #6538 | |
| | Staphylococcus aureus sub | Staphylococcus aureus sub sp. aureus #25923 |
| | sp. aureus #10832 | |
| | Staphylococcus aureus sub | |
| | sp. aureus #27734 | |
| Group 2: | Staphylococcus capitis sub | Staphylococcus capitis sub sp. capitis #35661 |
| | sp. capitis #27840 | |
| | Staphylococcus capitis sub | |
| | sp. capitis #27843 | |

| significance level: | 0.05 *: p < 0.05 **: p < 0.01 ***: p < 0.001 | | | | | |
|---|---|---|---|---|---|---|
| | Group 1 | | Group 2 | | | |
| | Mean | Standard | Mean | Standard | | |
| Lectins | Value | Deviation | Value | Deviation | T value | P value |
| GSL-II | 1.015 | 0.205 | 1.805 | 0.093 | −7.455 | 3.4E−04 *** |
| LPA | 0.231 | 0.156 | 0.791 | 0.039 | −7.644 | 7.5E−04 *** |
| rproBCA2 | 0.464 | 0.130 | 0.921 | 0.086 | −5.973 | 1.1E−03 ** |
| rOAA | 0.401 | 0.178 | 0.008 | 0.007 | 4.939 | 7.7E−03 ** |
| rproBCA1 | 0.397 | 0.153 | 0.985 | 0.182 | −4.681 | 0.011 * |
| Pro-CFA II | 0.612 | 0.233 | 0.181 | 0.052 | 3.983 | 0.012 * |
| rACG | 0.128 | 0.053 | 0.026 | 0.002 | 4.253 | 0.013 * |
| UEA-II | 0.228 | 0.088 | 0.595 | 0.128 | −4.382 | 0.020 * |
| AC-avranin | 0.314 | 0.104 | 0.133 | 0.060 | 3.127 | 0.020 * |
| algMPL | 0.368 | 0.216 | 0.009 | 0.003 | 3.714 | 0.021 * |
| PAA | 0.040 | 0.020 | 0.482 | 0.122 | −6.243 | 0.023 * |
| rAPI 144 | 0.481 | 0.202 | 0.142 | 0.127 | 2.923 | 0.027 * |
| CPA | 0.396 | 0.216 | 0.859 | 0.200 | −3.075 | 0.031 * |
| DSA | 0.056 | 0.034 | 0.545 | 0.169 | −4.973 | 0.035 * |
| rRSL | 0.250 | 0.090 | 0.517 | 0.119 | −3.352 | 0.036 * |
| CBA | 0.223 | 0.152 | 0.018 | 0.011 | 2.988 | 0.040 * |
| rCHA-1 | 0.473 | 0.175 | 0.930 | 0.222 | −3.040 | 0.046 * |

As is evident from the results shown in Table 22, it was possible to distinguish between Staphylococcus aureus at the logarithmic growth phase and Staphylococcus capitis at the logarithmic growth phase by using GSL-II, LPA, proBCA2, OAA, proBCA1, Pro-CFA II, ACG, UEA-II, AC-avranin, algMPL, PAA, API 144, CPA, DSA, RSL, CBA or, CHA-1.

### Example 21

### Selection of Lectins Capable of Distinguishing Between Staphylococcus epidermidis at Logarithmic Growth Phase and Staphylococcus capitis at Logarithmic Growth Phase

The above-described absorbance data was used to perform the significance test, and the lectins capable of distinguishing between Staphylococcus epidermidis at the logarithmic growth phase and Staphylococcus capitis at the logarithmic growth phase were selected. The obtained results together with the conditions of the significance test are shown in Table 23.

TABLE 23
S. epidermidis VS. S. capitis (Welch)

| Type of Test: | t test | |
|---|---|---|
| Group 1: | Staphylococcus epidermidis | Staphylococcus epidermidis #14990 |
| | #12228 | |
| | Staphylococcus epidermidis | |
| | #35547 | |
| Group 2: | Staphylococcus capitis sub | Staphylococcus capitis sub sp. capitis #35661 |
| | sp. capitis #27840 | |
| | Staphylococcus capitis sub | |
| | sp. capitis #27843 | |

| significance level: | 0.05 *: p < 0.05 **: p < 0.01 ***: p < 0.001 | | | | | |
|---|---|---|---|---|---|---|
| | Group 1 | | Group 2 | | | |
| | Mean | Standard | Mean | Standard | | |
| Lectins | Value | Deviation | Value | Deviation | T value | P value |
| rproBCA2 | 0.143 | 0.106 | 0.921 | 0.086 | −9.890 | 7.1E−04 *** |
| GSL-II | 1.178 | 0.062 | 1.805 | 0.093 | −9.764 | 1.2E−03 ** |
| rproBCA1 | 0.139 | 0.085 | 0.985 | 0.182 | −7.286 | 6.5E−03 ** |
| LPA | 0.121 | 0.126 | 0.791 | 0.039 | −8.816 | 7.2E−03 ** |
| LAA | 0.175 | 0.104 | 0.665 | 0.024 | −7.950 | 0.012 * |
| CPA | 0.113 | 0.073 | 0.859 | 0.200 | −6.065 | 0.015 * |
| rCHA-1 | 0.174 | 0.106 | 0.930 | 0.222 | −5.317 | 0.015 * |
| UEA-II | 0.068 | 0.033 | 0.595 | 0.128 | −6.901 | 0.015 * |
| rRSL | 0.098 | 0.046 | 0.517 | 0.119 | −5.686 | 0.016 * |
| SHA | 0.021 | 0.018 | 0.208 | 0.056 | −5.535 | 0.020 * |
| PAA | 0.014 | 0.003 | 0.482 | 0.122 | −6.670 | 0.022 * |

As is evident from the results shown in Table 23, it was possible to distinguish between Staphylococcus epidermidis at the logarithmic growth phase and Staphylococcus capitis at the logarithmic growth phase by using proBCA2, GSL-II, proBCA1, LPA, LAA, CPA, CHA-1, UEA-II, RSL, SHA, or PAA.

### Example 22

### Selection of Lectins Capable of Distinguishing between Staphylococcus hominis at Logarithmic Growth Phase and Other Staphylococci at Logarithmic Growth Phase

The plate immobilized with the 153 types of lectins was used to examine the binding of 16 types of bacteria described in Table 17 to the lectins according to the plate centrifugation. Note that for each bacterium the measurement was independently conducted three times (N=3) per lectin. Also, there was used data obtained by subtracting a measured value of Blank whose well of the plate was not immobilized with any lectin from a measured value of absorbance per lectin; and the statistical analysis was performed. Overall differences between the groups were verified by the one-way analysis of variance. Also, the difference between Staphylococcus hominis and the other bacteria was analyzed by the Dunnett's multiple comparison test. The obtained results together with the conditions of the significance test are shown in Tables 24 and 25.

TABLE 24
CFA
One-way analysis of variance

| P value | P < 0.0001 |
|---|---|
| P value summary | *** |
| Number of groups | 16 |
| F | 39.74 |
| R squared | 0.9491 |

| ANOVA Table | SS | df | MS |
|---|---|---|---|
| Treatment (between columns) | 0.3882 | 15 | 0.02588 |
| Residual (within columns) | 0.02084 | 32 | 0.0008511 |
| Total | 0.409 | 47 | |

| Dunnett's Multiple Comparison Test | Mean Diff | q value | Summary |
|---|---|---|---|
| S. hominis #27844 vs | 0.3386 | 16.25 | *** |
| S. aureus #6538 | | | |
| S. hominis #27844 vs | 0.3656 | 17.55 | *** |
| S. aureus #27217 | | | |
| S. hominis #27844 vs | 0.3527 | 16.93 | *** |
| S. aureus #25923 | | | |
| S. hominis #27844 vs | 0.3266 | 15.68 | *** |
| S. aureus #10832 | | | |
| S. hominis #27844 vs | 0.3449 | 16.56 | *** |
| S. aureus #27734 | | | |
| S. hominis #27844 vs | 0.2309 | 11.08 | *** |
| S. epidermidis #12228 | | | |
| S. hominis #27844 vs | 0.3323 | 15.95 | *** |
| S. epidermidis #14990 | | | |
| S. hominis #27844 vs | 0.3653 | 17.53 | *** |
| S. epidermidis #35547 | | | |
| S. hominis #27844 vs | 0.3633 | 17.44 | *** |
| S. capitis #27840 | | | |
| S. hominis #27844 vs | 0.3604 | 17.3 | *** |
| S. capitis #35661 | | | |
| S. hominis #27844 vs | 0.3606 | 17.31 | *** |
| S. capitis #27843 | | | |
| S. hominis #27844 vs | 0.3593 | 17.24 | *** |
| S. haemolyticus #29970 | | | |
| S. hominis #27844 vs | 0.36 | 17.28 | *** |
| E. coli #8739 | | | |
| S. hominis #27844 vs | 0.3673 | 17.63 | *** |
| B. subtilis #6833 | | | |
| S. hominis #27844 vs | 0.3703 | 17.77 | *** |
| P. aeruginosa #9027 | | | |

TABLE 25
BPL
One-way analysis of variance

| P value | P < 0.0001 |
|---|---|
| P value summary | *** |
| Number of groups | 16 |
| F | 29.84 |
| R squared | 0.9333 |

| ANOVA Table | SS | df | MS |
|---|---|---|---|
| Treatment (between columns) | 4.533 | 15 | 0.3022 |
| Residual (within columns) | 0.3241 | 32 | 0.01013 |
| Total | 4.858 | 47 | |
| Dunnett's Multiple Comparison Test | Mean Diff. | q value | Summary |
| S. hominis #27844 vs | 1.169 | 14.23 | *** |
| S. aureus #6538 | | | |
| S. hominis #27844 vs | 1.235 | 15.03 | *** |
| S. aureus #27217 | | | |
| S. hominis #27844 vs | 1.188 | 14.46 | *** |
| S. aureus #25923 | | | |
| S. hominis #27844 vs | 1.253 | 15.24 | *** |
| S. aureus #10832 | | | |
| S. hominis #27844 vs | 1.083 | 13.17 | *** |
| S. aureus #27734 | | | |
| S. hominis #27844 vs | 1.171 | 14.25 | *** |
| S. epidermis #12226 | | | |
| S. hominis #27844 vs | 1.195 | 14.55 | *** |
| S. epidermis #14990 | | | |
| S. hominis #27844 vs | 1.29 | 15.69 | *** |
| S. epidermis #35547 | | | |
| S. hominis #27844 vs | 1.041 | 12.67 | *** |
| S. capitis #27840 | | | |
| S. hominis #27844 vs | 0.8749 | 10.65 | *** |
| S. capitis #35661 | | | |
| S. hominis #27844 vs | 0.8223 | 10.01 | *** |
| S. capitis #27843 | | | |
| S. hominis #27844 vs | 1.19 | 14.48 | *** |
| S. haemolyticus #29970 | | | |
| S. hominis #27844 vs | 1.292 | 15.72 | *** |
| E. coli #8739 | | | |
| S. hominis #27844 vs | 1.238 | 15.06 | *** |
| B. subtilis #6633 | | | |
| S. hominis #27844 vs | 1.199 | 14.59 | *** |
| P. aeruginosa #9027 | | | |

As is evident from the results shown in Tables 24 and 25, it was possible to distinguish between Staphylococcus hominis at the logarithmic growth phase and the other Staphylococci at the logarithmic growth phase by using BPL or CFA (CFA1 and CFA2)

### Example 23

### Identification of Staphylococcus aureus by Lectin in Mixture with Other Bacteria

Even in the case where other bacteria (such as Staphylococcus epidermidis) are present in the mixture, the test was conducted to confirm that the food poisoning bacterium (Staphylococcus aureus) can be identified by the lectin in the same manner as that described above.

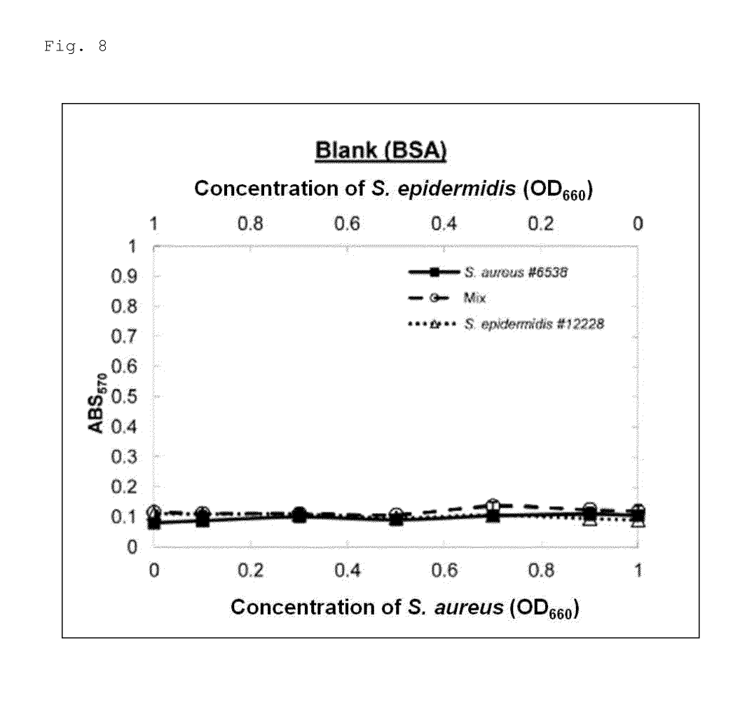

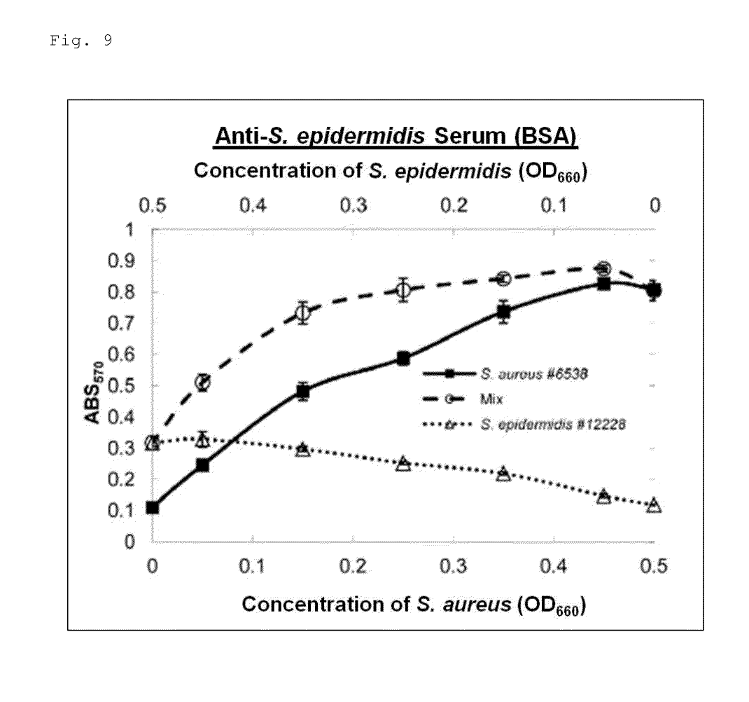

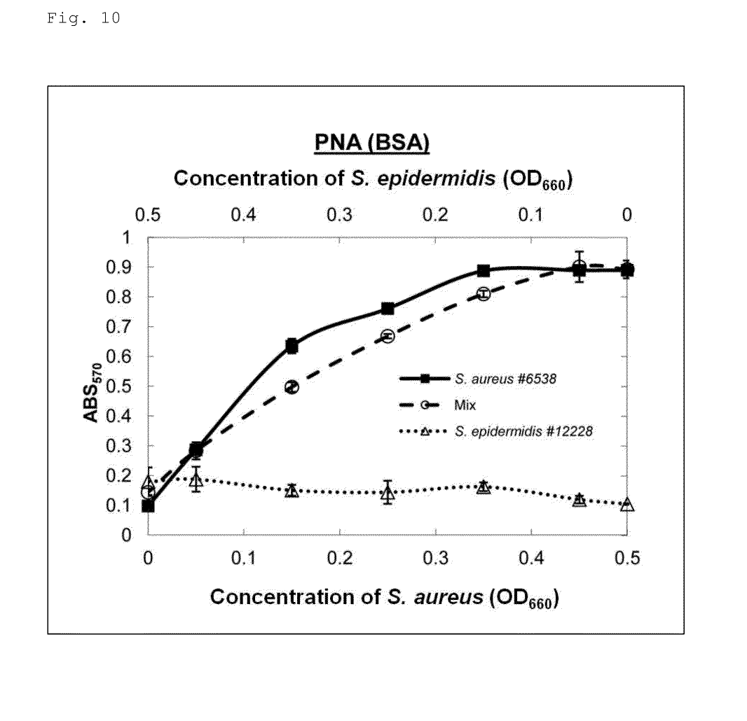

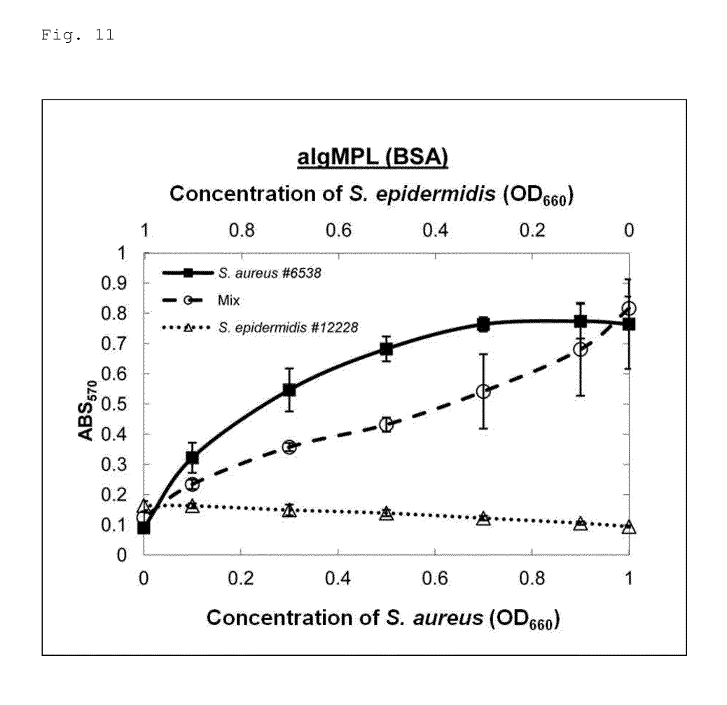

Specifically, the two types of lectins (PNA and algMPL) that proved to be able to identify the food poisoning bacterium were each used to ascertain that the each lectin alone can identify the food poisoning bacterium in the presence of plural bacteria in mixture: to this end, Staphylococcus aureus ATCC6538 as the food poisoning bacterium and the resident Staphylococcus epidermidis ATCC12228 as a resident bacterium were selected. The resident Staphylococcus epidermidis ATCC12228 was said to be difficult to be distinguished from Staphylococcus aureus. The verification of reaction by the lectin was conducted when the respective ones were mixed. The final concentration of each bacterium was fixed to a turbidity of 0.5 or 1 at a wavelength of 660 nm and mixing of the bacteria was conducted at an appropriate proportion. Note that each of the bacteria at the logarithmic growth phase was utilized to be suspended in 1% BSA/CM-TBS (TBS, 1% BSA, 1 mM CaCl₂, 1 mM MnCl₂) and was used to carry out the measurement by the plate centrifugation in the same manner as that described above. The results obtained are shown in FIGS. 8 to 11. Note that in FIGS. 8 to 11, the concentration of Staphylococcus aureus is arranged so as to become stronger from the left to the right of the graph (which refers to the horizontal axis under the graph of each figure) and that the concentration of Staphylococcus epidermidis is arranged so as to become stronger from the right to the left of the graph (which refers to the horizontal axis under the graph of each figure). The broken line also shows the results obtained when Staphylococcus aureus and Staphylococcus epidermidis were mixed at appropriate proportions to provide fixed final concentrations. Specifically, at the left end of each graph Staphylococcus epidermidis is 100%; and the right end of each graph Staphylococcus aureus is 100%; and in the middle of each graph they are 50%, respectively.

As is evident from the results shown in FIGS. 8 to 11, when each bacterium is allowed to react with the lectin plate, Staphylococcus aureus (solid line) shows a curve raising to the right in a concentration-dependent manner; and Staphylococcus epidermidis (dotted line) shows a curve declining to the right in a concentration-dependent manner. Further, in the case where Staphylococcus aureus and Staphylococcus epidermidis are mixed, the anti-S. epidermidis incapable of identifying the food poisoning bacterium showed nearly constant values in absorbance even when the mixing ratios were varied. On the other hand, it became evident that the lectins capable of identifying Staphylococcus aureus, such as PNA and algMPL, could detect Staphylococcus aureus in a concentration-dependent manner even in mixture with other bacteria within the genus. Particularly, with respect of PNA, the concentration response curve in the case of Staphylococcus aureus alone and the concentration response curve in the case of mixture with Staphylococcus epidermidis almost coincide with each other.

Accordingly, it was shown that the lectins according to the present invention (such as PNA and algMPL) allowed the food poisoning bacterium to be detected even in cases where the food poisoning bacterium (Staphylococcus aureus) and other bacteria (such as a resident Staphylococcus; Staphylococcus epidermidis) are in mixture.

### Example 24

### Identification of Staphylococcus aureus by Lectin in Food

It was to be confirmed that the food poisoning bacterium (Staphylococcus aureus) could be identified by the lectin(s). It was also to be shown from the viewpoint of practicality that the food poisoning bacterium could be conveniently and rapidly detected by the method of the present invention. To these ends, direct detection of the food poisoning bacterium in milk by the lectin(s) was attempted. Large amounts of lactooligosaccharide, glycoproteins, and glycolipids that possibly inhibit the binding between the lectins and bacteria are contained in milk; therefore, the testing in the milk can be “merkmal” in the identification of Staphylococcus aureus by the lectin(s) in foods.

The strains used are shown in Table 26. For each strain, cells at the late logarithmic growth phase were utilized and suspended in whole milk on the market for use. Among the lectins that were used in the logarithmic growth phase screening, 14 types of the commercially available lectins, 5 types of the purified lectins from natural extracts, and 17 types of recombinant lectins were used in a total of 36 types. Further, the measurement was carried out by the plate centrifugation in the same manner as that described above.

TABLE 26

| | Baacteria | ATCC No. | Type |
|---|---|---|---|
| 1 | Staphylococcus aureus | 6538 | food poisoning bacterium |
| | subsp. aureus | | |
| 2 | Staphylococcus epidermidis | 12228 | resident bacterium |
| 3 | Staphylococcus capitis | 27840 | resident bacterium |
| | subsp. capitis | | |
| 4 | Escherichia coli | 8739 | reference bacterium |
| 5 | Bacillus subtilis | 6623 | reference bacterium |
| | subsp. spizizenii | | |
| 6 | Psedomonas aeruginosa | 9027 | reference bacterium |

Note that for each bacterium the measurement was independently conducted three times (N=3) per lectin. Also, there was used data obtained by subtracting a measured value of Blank whose well of the plate was not immobilized with any lectin from a measured value of absorbance per lectin; and the statistical analysis was performed. Overall differences between the groups were verified by the one-way analysis of variance. Also, the difference between Staphylococcus aureus and the other bacteria was analyzed by the Dunnett's multiple comparison test. The obtained results together with the conditions of the significance test are shown in Tables 27 to 39.

TABLE 27
algMPL
One-way analysis of variance

| P value | P < 0.0001 |
|---|---|
| P value summary | *** |
| Number of groups | 6 |
| F | 99.58 |
| R squared | 0.9765 |

| ANOVA Table | SS | df | MS |
|---|---|---|---|
| Treatment (between columns) | 0.6872 | 5 | 0.1374 |
| Residual (within columns) | 0.01658 | 12 | 0.00138 |
| Total | 0.7038 | 17 | |
| Dunnett's Multiple Comparison Test | Mean Diff. | q value | Summary |
| S. aureus #6538 vs | 0.521 | 17.18 | *** |
| S. epidermidis #12228 | | | |
| S. aureus #6538 vs | 0.485 | 15.99 | *** |
| S. capitis #27840 | | | |
| S. aureus #6538 vs | 0.5337 | 17.59 | *** |
| E. coli #8739 | | | |
| S. aureus #6538 vs | 0.5497 | 18.12 | *** |
| B. subtilis #6633 | | | |
| S. aureus #6538 vs | 0.3167 | 10.44 | *** |
| P. aeruginosa #9027 | | | |

TABLE 28
PNA
One-way analysis of variance

| P value | P < 0.0001 |
|---|---|
| P value summary | *** |
| Number of groups | 6 |
| F | 73.76 |
| R squared | 0.9685 |

| ANOVA Table | SS | df | MS |
|---|---|---|---|
| Treatment (between columns) | 2.35 | 5 | 0.4701 |
| Residual (within columns) | 0.07648 | 12 | 0.006374 |
| Total | 2.427 | 17 | |
| Dunnett's Multiple Comparison Test | Mean Diff. | q value | Summary |
| S. aureus #6538 vs | 0.9033 | 13.88 | *** |
| S. epidermidis #12228 | | | |
| S. aureus #6538 vs | 0.8563 | 13.14 | *** |
| S. capitis #27840 | | | |
| S. aureus #6538 vs | 0.93 | 14.27 | *** |
| E. coli #8739 | | | |
| S. aureus #6538 vs | 0.9207 | 14.12 | *** |
| B. subtilis #6633 | | | |
| S. aureus #6538 vs | 0.3293 | 5.052 | ** |
| P. aeruginosa #9027 | | | |

TABLE 29
DBA
One-way analysis of variance

| P value | P < 0.0001 |
|---|---|
| P value summary | *** |
| Number of groups | 6 |
| F | 35.02 |
| R squared | 0.9359 |

| ANOVA Table | SS | df | MS |
|---|---|---|---|
| Treatment (between columns) | 1.359 | 5 | 0.2737 |
| Residual (within columns) | 0.0938 | 12 | 0.007816 |
| Total | 1.462 | 17 | |
| Dunnett's Multiple Comparison Test | Mean Diff. | q value | Summary |
| S. aureus #6538 vs | 0.757 | 10.49 | *** |
| S. epidermidis #12228 | | | |
| S. aureus #6538 vs | 0.677 | 9.378 | *** |
| S. capitis #27840 | | | |
| S. aureus #6538 vs | 0.7737 | 10.72 | *** |
| E. coli #8739 | | | |
| S. aureus #6538 vs | 0.7587 | 10.51 | *** |
| B. subtilis #6633 | | | |
| S. aureus #6538 vs | 0.481 | 6.663 | *** |
| P. aeruginosa #9027 | | | |

TABLE 30
rTachylectin-3
One-way analysis of variance

| P value | P < 0.0001 |
|---|---|
| P value summary | *** |
| Number of groups | 6 |
| F | 83.4 |
| R squared | 0.972 |

| ANOVA Table | SS | df | MS |
|---|---|---|---|
| Treatment (between columns) | 2.402 | 5 | 0.4804 |
| Residual (within columns) | 0.06912 | 12 | 0.00576 |
| Total | 2.471 | 17 | |
| Dunnett's Multiple Comparison Test | Mean Diff. | q value | Summary |
| S. aureus #6538 vs | 0.9767 | 15.76 | *** |
| S. epidermidis #12228 | | | |
| S. aureus #6538 vs | 0.9067 | 14.63 | *** |
| S. capitis #27840 | | | |
| S. aureus #6538 vs | 1.008 | 16.27 | *** |
| E. coli #8739 | | | |
| S. aureus #6538 vs | 1.027 | 16.58 | *** |
| B. subtilis #6633 | | | |
| S. aureus #6538 vs | 0.613 | 9.892 | *** |
| P. aeruginosa #9027 | | | |

TABLE 31
rTPL-1
One-way analysis of variance

| P value | P < 0.0001 |
|---|---|
| P value summary | *** |
| Number of groups | 6 |
| F | 150.3 |
| R squared | 0.9843 |

| ANOVA Table | SS | df | MS |
|---|---|---|---|
| Treatment (between columns) | 1.705 | 5 | 0.341 |
| Residual (within columns) | 0.02722 | 12 | 0.002268 |
| Total | 1.732 | 17 | |
| Dunnett's Multiple Comparison Test | Mean Diff | q value | Summary |
| S. aureus #6538 vs | 0.8333 | 21.43 | *** |
| S. epidermidis #12228 | | | |
| S. aureus #6538 vs | 0.808 | 20.78 | *** |
| S. capitis #27840 | | | |
| S. aureus #6538 vs | 0.8633 | 22.2 | *** |
| E. coli #8739 | | | |
| S. aureus #6538 vs | 0.8607 | 22.13 | *** |
| B. subtilis #6633 | | | |
| S. aureus #6538 vs | 0.6747 | 17.35 | *** |
| P. aeruginosa #9027 | | | |

TABLE 32
GSL-II
One-way analysis of variance

| P value | P < 0.0001 |
|---|---|
| P value summary | *** |
| Number of groups | 6 |
| F | 34.5 |
| R squared | 0.935 |

| ANOVA Table | SS | df | MS |
|---|---|---|---|
| Treatment (between columns) | 3.126 | 5 | 0.6252 |
| Residual (within columns) | 0.2175 | 12 | 0.01812 |
| Total | 3.343 | 17 | |
| Dunnett's Multiple Comparison Test | Mean Diff. | q value | Summary |
| S. aureus #6538 vs | 0.787 | 7.16 | *** |
| S. epidermidis #12228 | | | |
| S. aureus #6538 vs | 0.6967 | 6.338 | *** |
| S. capitis #27840 | | | |
| S. aureus #6538 vs | 1.197 | 10.89 | *** |
| E. coli #8739 | | | |
| S. aureus #6538 vs | 1.266 | 11.52 | *** |
| B. subtilis #6633 | | | |
| S. aureus #6538 vs | 0.6483 | 5.898 | *** |
| P. aeruginosa #9027 | | | |

TABLE 33
rBML11b
One-way analysis of variance

| P value | P < 0.0001 |
|---|---|
| P value summary | *** |
| Number of groups | 6 |
| F | 49.62 |
| R squared | 0.9539 |

| ANOVA Table | SS | df | MS |
|---|---|---|---|
| Treatment (between columns) | 0.5198 | 5 | 0.104 |
| Residual (within columns) | 0.02514 | 12 | 0.002095 |
| Total | 0.545 | 17 | |
| Dunnett's Multiple Comparison Test | Mean Diff. | q value | Summary |
| S. aureus #6538 vs | 0.3237 | 8.661 | *** |
| S. epidermidis #12228 | | | |
| S. aureus #6538 vs | 0.2423 | 6.484 | *** |
| S. capitis #27840 | | | |
| S. aureus #6538 vs | 0.4773 | 12.77 | *** |
| E. coli #8739 | | | |
| S. aureus #6538 vs | 0.504 | 13.49 | *** |
| B. subtilis #6633 | | | |
| S. aureus #6538 vs | 0.2193 | 5.869 | *** |
| P. aeruginosa #9027 | | | |

TABLE 34
rBCL11
One-way analysis of variance

| P value | P < 0.0001 |
|---|---|
| P value summary | *** |
| Number of groups | 6 |
| F | 22.61 |
| R squared | 0.904 |

| ANOVA Table | SS | df | MS |
|---|---|---|---|
| Treatment (between columns) | 0.8936 | 5 | 0.1787 |
| Residual (within columns) | 0.09485 | 12 | 0.007904 |
| Total | 0.9884 | 17 | |
| Dunnett's Multiple Comparison Test | Mean Diff. | q value | Summary |
| S. aureus #6538 vs | 0.3793 | 5.226 | *** |
| S. epidermidis #12228 | | | |
| S. aureus #6538 vs | 0.4623 | 6.369 | *** |
| S. capitis #27840 | | | |
| S. aureus #6538 vs | 0.6507 | 8.963 | *** |
| E. coli #8739 | | | |
| S. aureus #6538 vs | 0.6763 | 9.317 | *** |
| B. subtilis #6633 | | | |
| S. aureus #6538 vs | 0.4407 | 6.071 | *** |
| P. aeruginosa #9027 | | | |

TABLE 35
rBML11c
One-way analysis of variance

| P value | P < 0.0001 |
|---|---|
| P value summary | *** |
| Number of groups | 6 |
| F | 50.56 |
| R squared | 0.9547 |

| ANOVA Table | SS | df | MS |
|---|---|---|---|
| Treatment (between columns) | 0.4738 | 5 | 0.09476 |
| Residual (within columns) | 0.02249 | 12 | 0.001874 |
| Total | 0.4963 | 17 | |
| Dunnett's Multiple Comparison Test | Mean Diff. | q value | Summary |
| S. aureus #6538 vs | 0.371 | 10.5 | *** |
| S. epidermidis #12228 | | | |
| S. aureus #6538 vs | 0.34 | 9.619 | *** |
| S. capitis #27840 | | | |
| S. aureus #6538 vs | 0.4227 | 11.96 | *** |
| E. coli #8739 | | | |
| S. aureus #6538 vs | 0.4413 | 12.49 | *** |
| B. subtilis #6633 | | | |
| S. aureus #6538 vs | 0.1337 | 3.782 | * |
| P. aeruginosa #9027 | | | |

TABLE 36
rTachylectin-2
One-way analysis of variance

| P value | P < 0.0001 |
|---|---|
| P value summary | *** |
| Number of groups | 6 |
| F | 284.9 |
| R squared | 0.9916 |

| ANOVA Table | SS | df | MS |
|---|---|---|---|
| Treatment (between columns) | 4.967 | 5 | 0.9934 |
| Residual (within columns) | 0.04184 | 12 | 0.003487 |
| Total | 5.009 | 17 | |
| Dunnett's Multiple Comparison Test | Mean Diff | q value | Summary |
| S. aureus #6538 vs | 0.1673 | 3.471 | * |
| S. epidermidis #12228 | | | |
| S. aureus #6538 vs | 0.692 | 14.35 | *** |
| S. capitis #27840 | | | |
| S. aureus #6538 vs | 1.261 | 26.16 | *** |
| E. coli #8739 | | | |
| S. aureus #6538 vs | 1.305 | 27.07 | *** |
| B. subtilis #6633 | | | |
| S. aureus #6538 vs | 1.177 | 24.42 | *** |
| P. aeruginosa #9027 | | | |

TABLE 37
PVL
One-way analysis of variance

| P value | P < 0.0001 |
|---|---|
| P value summary | *** |
| Number of groups | 6 |
| F | 38.12 |
| R squared | 0.9408 |

| ANOVA Table | SS | df | MS |
|---|---|---|---|
| Treatment (between columns) | 2.049 | 5 | 0.4098 |
| Residual (within columns) | 0.129 | 12 | 0.01075 |
| Total | 2.178 | 17 | |
| Dunnett's Multiple Comparison Test | Mean Diff. | q value | Summary |
| S. aureus #6538 vs | 0.6107 | 7.213 | *** |
| S. epidermidis #12228 | | | |
| S. aureus #6538 vs | 0.725 | 8.563 | *** |
| S. capitis #27840 | | | |
| S. aureus #6538 vs | 1.003 | 11.85 | *** |
| E. coli #8739 | | | |
| S. aureus #6538 vs | 0.9997 | 11.81 | *** |
| B. subtilis #6633 | | | |
| S. aureus #6538 vs | 0.7647 | 9.032 | *** |
| P. aeruginosa #9027 | | | |

TABLE 38
LBA
One-way analysis of variance

| P value | P < 0.0001 |
|---|---|
| P value summary | *** |
| Number of groups | 6 |
| F | 50.09 |
| R squared | 0.9543 |

| ANOVA Table | SS | df | MS |
|---|---|---|---|
| Treatment (between columns) | 1.985 | 5 | 0.3969 |
| Residual (within columns) | 0.0951 | 12 | 0.007925 |
| Total | 2.08 | 17 | |
| Dunnett's Multiple Comparison Test | Mean Diff. | q value | Summary |
| S. aureus #6538 vs | 0.884 | 12.16 | *** |
| S. epidermidis #12228 | | | |
| S. aureus #6538 vs | 0.8197 | 11.28 | *** |
| S. capitis #27840 | | | |
| S. aureus #6538 vs | 0.9157 | 12.6 | *** |
| E. coli #8739 | | | |
| S. aureus #6538 vs | 0.9217 | 12.68 | *** |
| B. subtilis #6633 | | | |
| S. aureus #6538 vs | 0.5247 | 7.218 | *** |
| P. aeruginosa #9027 | | | |

TABLE 39
rUPL-1
One-way analysis of variance

| P value | P < 0.0001 |
|---|---|
| P value summary | *** |
| Number of groups | 6 |
| F | 57.23 |
| R squared | 0.9597 |

| ANOVA Table | SS | df | MS |
|---|---|---|---|
| Treatment (between columns) | 0.2249 | 5 | 0.04498 |
| Residual (within columns) | 0.009433 | 12 | 0.0007861 |
| Total | 0.2344 | 17 | |
| Dunnett's Multiple Comparison Test | Mean Diff. | q value | Summary |
| S. aureus #6538 vs | 0.1107 | 4.834 | ** |
| S. epidermidis #12228 | | | |
| S. aureus #6538 vs | 0.124 | 5.417 | *** |
| S. capitis #27840 | | | |
| S. aureus #6538 vs | 0.1613 | 7.048 | *** |
| E. coli #8739 | | | |
| S. aureus #6538 vs | 0.1767 | 7.717 | *** |
| B. subtilis #6633 | | | |
| S. aureus #6538 vs | −0.144 | 6.29 | *** |
| P. aeruginosa #9027 | | | |

As is evident from the results shown in Tables 27 to 39, it was possible to identify the food poisoning bacterium (Staphylococcus aureus) that is present in the milk at least by using algMPL, PNA, DBA, Tachylectin-3, TPL-1, GSL-II, BML11b, BCL11, BML11c, Tachylectin-2, PVL, LBA, or UPL-1.

### Example 25

### Identification of Staphylococcus aureus in Mixture with Other Bacteria in Food

In order to confirm that the lectin(s) according to the present invention could identify the food poisoning bacterium even in the situation where a plurality of bacteria were present in mixture in a food (such as milk), Staphylococcus aureus ATCC6538 as the food poisoning bacterium and the resident Staphylococcus epidermidis ATCC12228 as a resident bacterium were selected, and the test was conducted in the same manner as that described in Example 23. Staphylococcus epidermidis ATCC12228 was said to be difficult to be distinguished from Staphylococcus aureus. Note that for each bacterium, cells at the late logarithmic growth phase were utilized and suspended in whole milk on the market for use; and the measurement was conducted by the plate centrifugation.

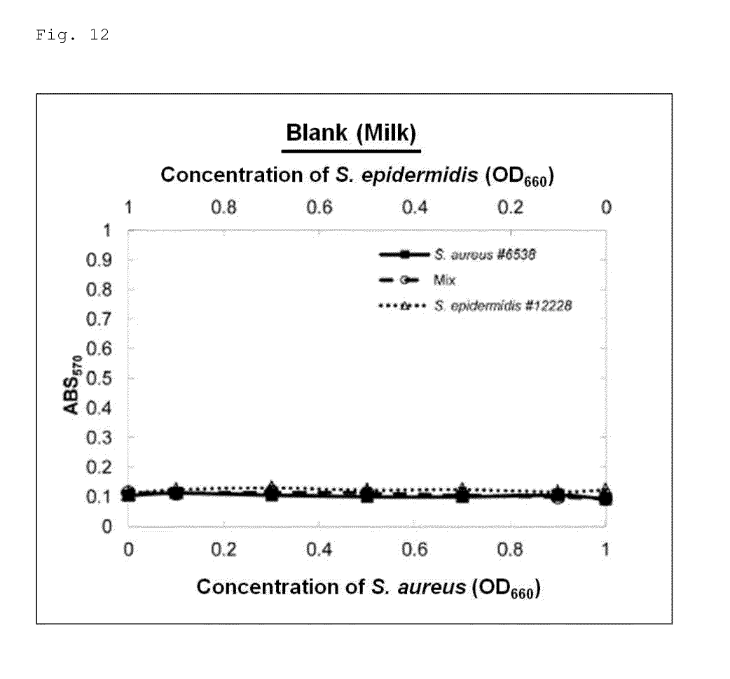

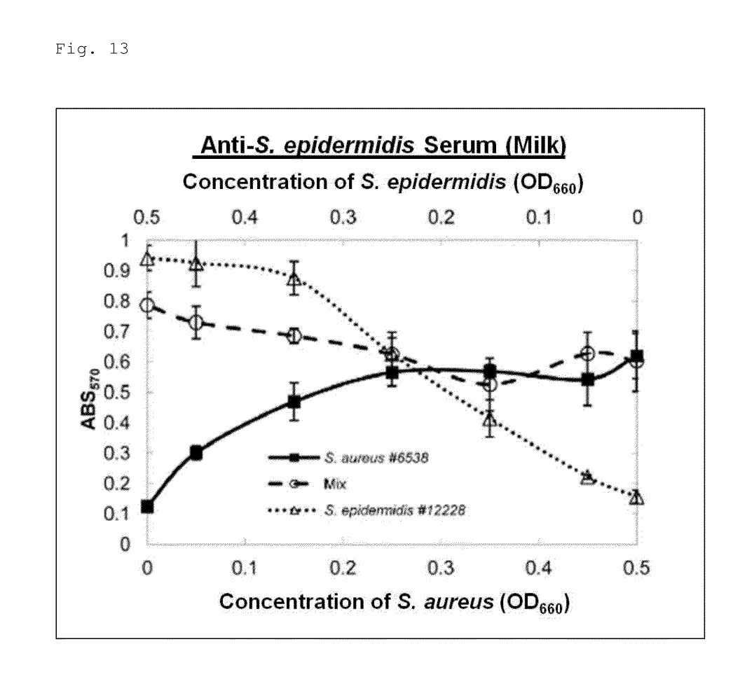

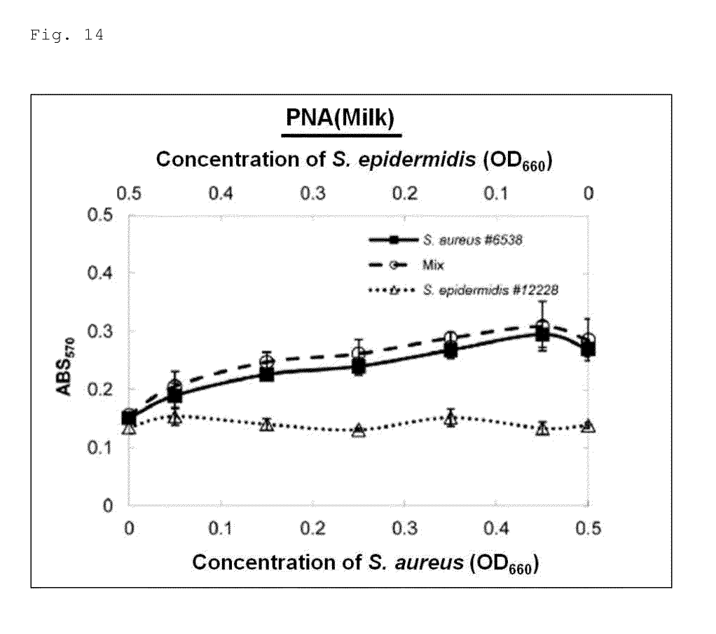

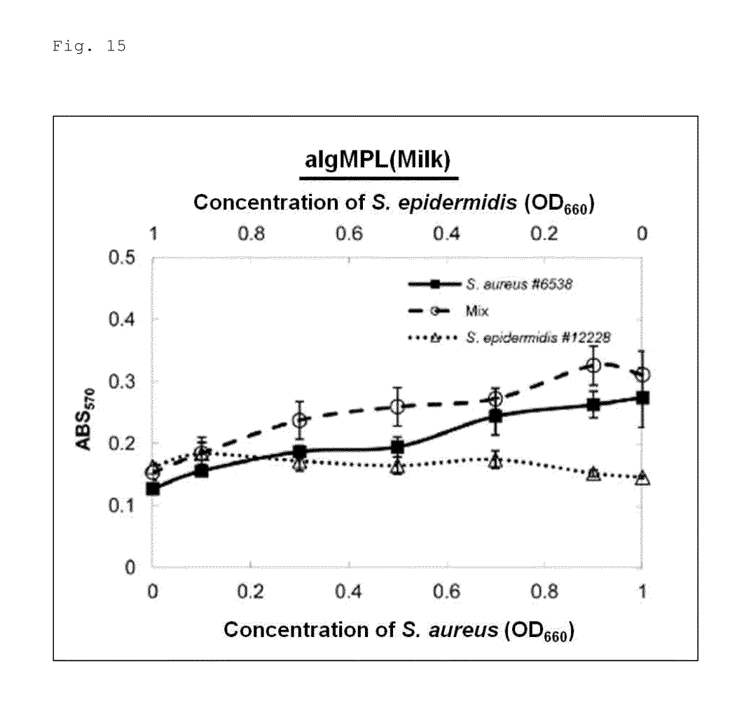

The obtained results are shown in Tables 12 to 15. Note that in FIGS. 12 to 15, the concentration of Staphylococcus aureus is arranged so as to become stronger from the left to the right of the graphs (which refers to the horizontal axis under the graph of each figure) and that the concentration of Staphylococcus epidermidis is arranged so as to become stronger from the right to the left of the graph (which refers to the horizontal axis under the graph of each figure). The broken line also shows the results obtained when Staphylococcus aureus and Staphylococcus epidermidis were mixed at appropriate proportions to provide fixed final concentrations. Specifically, at the left end of each graph Staphylococcus aureus is 100%; and the right end of each graph Staphylococcus aureus is 100%; and in the middle of each graph they are 50%, respectively.

As is evident from the results shown in FIGS. 12 to 15, in the case where Staphylococcus aureus and Staphylococcus epidermidis are mixed, the anti-S. epidermidis incapable of identifying the food poisoning bacterium showed nearly constant values in absorbance even when the mixing ratios were varied. On the other hand, it became evident that the lectins capable of identifying Staphylococcus aureus, such as PNA and algMPL, could detect Staphylococcus aureus in a concentration-dependent manner even in mixture with other bacteria within the genus in the milk. Particularly, with respect of PNA in the milk, the concentration response curve in the case of Staphylococcus aureus alone and the concentration response curve in the case of mixture with Staphylococcus epidermidis almost coincide with each other.

Accordingly, it was shown that the lectins according to the present invention (such as PNA and algMPL) allowed the food poisoning bacterium to be detected even in cases where the food poisoning bacterium (Staphylococcus aureus) and other bacteria (such as the resident Staphylococcus; Staphylococcus epidermidis) are in mixture in a food (such as milk).

As stated above, the binding affinities between many types of lectins and the bacteria belonging to the genus Staphylococcus were examined. Consequently, it has became evident that the species within the genus Staphylococcus can be distinguished by Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, algMPL, or algCSA.

Note that these lectins are derived from organisms described below.

Tachylectin-2 (Tachypleus tridentatus lectin 2) derived from Japanese horseshoe crab (Tachypleus tridentatus);
LEL (Lycopersicon esculentum lectin) derived from tomato (lycopersicon esculentum);
KAA1 (Kappaphycus alvarezii agglutinin 1) derived from Kappaphycus alvarezii (formerly Eucheuma cottonii);
BCL11 (Bryopsis corticulans 11 kDa lectin) derived from Bryopsis corticulans;
CBA (Codium barbatum agglutinin) derived from Codium barbatum;
HAA (Helix aspersa agglutinin) derived from Helix aspersa;
HPA (Helix pomatia agglutinin) derived from Helix pomatia;
STL (Solanum tuberosum lectin) derived from potato (Solanum tuberosum);
proBCA1 (Boodlea coacta agglutinin 1 precursor) and proBCA2 (Boodlea coacta agglutinin 2 precursor) derived from Boodlea coacta;
ULL (Ulva limnetica lectin-like protein) derived from Ulva limnetica;
DSA (Datura stramonium agglutinin) derived from Datura stramonium;
PWM (pokeweed mitogen) derived from pokeweed (Phytolacca americana);
UDA (Urtica dioica agglutinin) derived from Urtica dioica;
WFL (Wisteria floribunda lectin) derived from Wisteria floribunda;
hypninA3 (Hypnea japonica lectin A3) derived from Hypnea japonica;
Tachylectin-3 (Tachypleus tridentatus lectin 3) derived from Japanese horseshoe crab (Tachypleus tridentatus);
OAA (Oscillatoria agardhii agglutinin) derived from Oscillatoria agardhii (Cyanobacteria);
PNA (Arachis hypogaea agglutinin, peanut agglutinin) derived from peanut;
TL (Tulipa lectin) derived from tulip;
ACG (Agrocybe cylindracea galectin) derived from Agrocybe cylindracea;
AC-avranin (Avrainvillea capituliformis-avranin) derived from Avrainvillea capituliformis (green algae);
MOA (Marasmium oreades agglutinin) derived from fairy ring mushroom (Marasmium oreades);
AP1 144 (Axinella polypoides lectin 144) derived from Axinella polypoides;
CV-N (Cyanovirin-N) derived from Nostoc ellipsosporum (cyanobacteria);
PMA (Polygonatum multiflorum agglutinin) derived from Polygonatum multiflorum;
GSL-II (Griffonia simplicifolia lectin) derived from Griffonia simplicifolia;
Garlic lectin (Allium sativum lectin) derived from garlic;
PAA (Perseau americana agglutinin) derived from avocado;
UEA-II (Ulex europaeus agglutinin-II) derived from Ulex europaeus;
RSL (Ralstonia solanacearum lectin) derived from Ralstonia solanacearum;
CPA (Cicer arietiunum agglutinin) derived from Cicer arietiunum;
CHA-1 (Cepaea hortensis agglutinin) derived from Cepaea hortensis;
LAA (Laburnum alpinum agglutinin) derived from Laburnum alpinum;
SHA (Salvia horminum agglutinin) derived from Salvia horminum;
LPA (Limulus polyphemus agglutinin) derived from Atlantic horseshoe crab;
DBA (Dolichos biflorus agglutinin) derived from Dolichos biflorus;
TPL-1 (Tachypleus plasma lectin 1) derived from Japanese horseshoe crab (Tachypleus tridentatus);
BML11b [Bryopsis maxima 11 kDa lectin b)], BML11b [Bryopsis maxima 11 kDa lectin c)] derived from Bryopsis maxima;
PVL (Psathyrella velutina lectin) derived from weeping widow (Psathyrella velutina);
LBA (Phaseolus lunatus agglutinin) derived from lima bean;
UPL-1 (Ulva pertusa lectin 1) derived from Ulva pertusa;
BPL (Bauhinia purpurea lectin) derived from Bauhinia purpurea;
CFA1 (Codium fragile agglutinin 1), CFA2 (Codium fragile agglutinin 2) derived from Codium fragile;
BanLec (Banana lectin) derived from Taiwan banana (Musa acuminata);
BCL11d (Bryopsis corticulans 11 kDa lectin d) derived from Bryopsis corticulans;
FVE (Flammulina velutipes edible) derived from Flammulina velutipes;
CLA (Codium latum agglutinin): derived from Codium latum;
Pro-CFA I (Pronase-treatment dependent Carpopeltis flabellate agglutinin I), Pro-CFA II (Pronase-treatment dependent Carpopeltis flabellate agglutinin II): derived from Polyopes prolifera;
MPA1 (Meristotheca papulosa agglutinin 1), MPA2 (Meristotheca papulosa agglutinin 2): derived from Meristotheca papulosa;
algMPL (MPL, Meristotheca papulosa lectin); derived from Meristotheca papulosa; and
algCSA (CSA, Codium subtubulosum agglutinin): derived from Codium subtubulosum.

Among these lectins, KAA1, BCL11, BCL11, BML11c, CBA, BCL11d, CFA1, CFA2, CLA, MPA1, MPA2, and AC-avranin were extracted, isolated, and purified or their full-length amino acid sequence and base sequences were determined by the present inventors. The methods for isolating or cloning these lectins are shown below.

### Example 26

### cDNA Cloning of KAA1

Plant RNA Isolation Reagent (manufactured by Life Technologies Corporation) was used to extract a total RNA from the algal thallus of Kappaphycus alvarezii (formally, Eucheuma cottonii) that was stored in an RNA stabilized solution (manufactured by Life Technologies Corporation; RNAlater) at −20° C. Thereafter, the mRNA was purified with NucleoTrap mRNA (manufactured by Macherey-Nagel GmbH & Co. KG) and a full-length cDNA was further prepared with GeneRacer Kit (manufactured by Life Technologies Corporation).

By referring to the amino acid sequences of the known portions of ESA2 and KAA derived from Eucheuma serra as a related species whose full-length amino acid was known, degenerate primers, KAA_{—}5′ RACE_d_R1, KAA_{—}5′ RACE_d_R2, and KAA_{—}3′ RACE_d_F1 were designed. A solution of the full-length cDNA derived from K. alvarezii was used as a template and subjected to RACE (Rapid Amplification of cDNAEnds). First, as for 5′RACE, a primer pair of KAA_{—}5′ RACE_d_R2 and GeneRacer_{—}5′_Primer, and Blend Taq DNA polymerase (manufactured by Toyobo Co., Ltd.) were used to perform PCR. Note that the composition of the PCR reaction solution (50 μl) is as follows: 1× Blend Taq buffer; dNTP mix 10 nmol, GeneRacer_{—}5′_Primer 30 pmol, KAA_{—}5′RACE_d_R2 250 pmol, the 10-fold diluted full-length cDNA solution 1 μl, and Blend Taq DNA polymerase 1.25 U. The reaction conditions of PCR were also such that after thermal denaturation at 94° C. for 3 min, 35 cycles of thermal denaturation at 94° C. for 30 sec, annealing at 64° C. for 30 sec, and elongation at 72° C. for 1 min were conducted and that finally, reaction was completed at 72° C. for 5 min.

Further, after diluting the PCR reaction solution 100 times, this was used as a template and was subjected to Nested PCR using a primer pair of KAA_{—}5′ RACE_d_R1 and GeneRacer_{—}5′_Nested_Primer. Note that the composition of the PCR reaction solution (50 μl) is as follows: 1× Blend Taq buffer; dNTP mix 10 nmol, GeneRacer_{—}5′_Nested_Primer 10 pmol, KAA_{—}5′RACE_d_R1 250 pmol, the 100-fold diluted PCR reaction solution 1 μl, and Blend Taq DNA polymerase 1.25 U. The reaction conditions of PCR were also such that the annealing temperature was set at 58° C. and PCR was performed in the same manner as that described above.

Next, the obtained amplified product was subcloned into a pGEM-T Easy vector (manufactured by Promega Corporation), and then it was subjected to base sequencing by using BigDye Terminator Cycle Sequencing Kit Ver. 3.1 and ABI 3130×1 DNA sequencer (manufactured by Life Technologies Corporation).

Next, as for 3′RACE, a primer pair of KAA_{—}3′ RACE_d_F1 and GeneRacer_{—}3′_Primer was used to perform PCR in the same manner as that described above, and the obtained amplified product was subjected to base sequencing. Primers KAA_{—}5′End_F and KAA1_{—}3′End_R were designed, which were specific to 5′- and 3′-terminal sequences revealed by the 5′- and 3′-RACE. These were made a primer pair and it was subjected to PCR using KOD FX Neo DNA polymerase (manufactured by Toyobo Co., Ltd.). Note that the composition of PCR reaction solution and the reaction conditions of PCR followed the attached user's manual of the DNA polymerase to perform PCR. The obtained amplified product was then subjected to base sequencing and the full-length cDNA sequence of KAA1 was revealed. The obtained base sequence is shown in SEQ ID NO:34 and the amino acid sequence based on the obtained base sequence is shown in SEQ ID NO:3. The base sequences of the primers used in Example 26 are also shown in Table 40.

TABLE 40

| Primers | Sequence (5′-3′) | SEQ ID NO. |
|---|---|---|
| GeneRacer_5′_Primer^{a} | CGACTGGAGCACGAGGACACTGA | 18 |
| GeneRacer_5′_Nested_Primer^{a} | GGACACTGACATGGACTGAAGGAGTA | 19 |
| GeneRacer_3′_Primer^{a} | GCTGTCAACGATACGCTACGTAACG | 20 |
| GeneRacer_3′_Nested_Primer^{a} | CGCTACGTAACGGCATGACAGTG | 21 |
| KAA-3′RACE-d-F1 | AYCAITAYAAYGTIGARAAYCARTGGGG | 22 |
| KAA-5′RACE-d-R1 | AYTGRTTYTCIACRTTRTAITGRTC | 23 |
| KAA-5′RACE-d-R2 | ATIGGICCYTCICCYTTRTAYTGC | 24 |
| KAA1_5′End_F | ATAGCTGAGTCAAGTTACACCAAC | 25 |
| KAA1_3′End_F | AGAGGGTGATCACGTTTTTAC | 26 |
| BOL11_5′RACE_dc_R1 | CCCCGGTCCCCCARICCYTTIAC | 27 |
| BOL11_3′RACE_F1 | CACCTCCGCTTCTACTCCTG | 28 |
| BOL11_5′End_F | ATTTGTTGCTATTCTCTGCACTGC | 29 |
| BOL11_3′End_R | CAACGCACTAACAAGCGTTAC | 30 |
| BOL11_like_common_R1 | CTCICTGGCITIGITCTGIGC | 31 |
| BML11 b_5′End_F | ACGGATACTTCTGGCTGCA | 32 |
| BML11 c_5′End_F | ATCCGATCTACACTTCGCGA | 33 |
| CEA_d_F1 | ACICAYGGIATHAARAAYGA | 74 |
| CEA_d_F2 | AAYGAYTGYGGIGTICCIGT | 75 |
| CBA_R1 | TCCAAGCAGCATACGAACAC | 76 |
| CBA_R2 | TCATCAGTCCCAGTCCAACA | 77 |
| BCL11d_3′ End_R | CGCACGGAAAGAAAAACCGT | 78 |
| CFA_5′RACE_R1 | TCRTAYTTIACRTCYTGIC | 79 |
| CFA_5′RACE_R2 | TAIGGYTTRTCRAAIACDATIGG | 80 |
| CFA1_3′RACE_R2 | GGGATCGTTCAAGAGTCAGG | 81 |
| CFA2_3′RACE_F | ATTGTTAAAGAGTCAGGCA | 82 |
| CLA_d_F1 | GCIYTICAYGTIACIYTIAC | 83 |
| CLA_d_F2 | ACIYTIACIGCIGAYACIGG | 84 |
| CLA_3′ End_R | GTTGGAATTTAGATTGTTGACTTAC | 85 |
| KAA_common_F1 | AGAACCAGTGGGGAGGATCT | 86 |
| KAA_3′ RACE_d_F2 | ARTAYAARGGIGARGGICCIATHGG | 87 |
| MPA1_R1 | TGTGCCTTCAAGGTTCTTCC | 88 |
| MPA2_R1 | TATGCGTCGAAGTCACCAAC | 89 |

Tm values are calculated according to the Nearest Neighbor method, as described in Rychlik W. et al., Nucleic Acids Res., 1990, Vol. 18, No. 21, pp. 6409-6412.
The mixed bases are denoted by IUB codes. Specifically, I represents inosine; R represents A or G; Y represents C or T; H represents A or T; and D represents not-C.
The primers whose names are affixed with “a” should be referred to the GeneRacer Kit (manufactured by Life Technologies Inc.).

### Example 27

### cDNA Cloning of BCL11

Plant RNA Isolation Reagent was used to extract a total RNA from the algal thallus of Bryopsis corticulans that was stored in RNAlater at −20° C. Thereafter, mRNA was purified with NucleoTrap mRNA and a full-length cDNA was further prepared with GeneRacer Kit.

Based on the known N-terminal amino acid sequence of BCL11, a degenerate primer BCL11_{—}5′ RACE_dc_R1 was designed according to CODEHOP program. A solution of the full-length cDNA derived from Bryopsis corticulans was subjected as a template to RACE. First, as for 5′RACE, a primer pair of BCL11_{—}5′ RACE_dc_R1 and GeneRacer_{—}5′_Primer, and Blend Taq DNA polymerase were used to perform PCR. Note that the composition of the PCR reaction solution (50 μl) is as follows: 1× Blend Taq buffer; dNTP mix 10 nmol, GeneRacer_{—}5′_Primer 30 pmol, BCL11_{—}5′RACE_dc_R1 250 pmol, the 10-fold diluted full-length cDNA solution 1 μl, and Blend Taq DNA polymerase 1.25 U. The reaction conditions of PCR were also such that after thermal denaturation at 94° C. for 3 min, 35 cycles of thermal denaturation at 94° C. for 30 sec, annealing at 50° C. for 30 sec, and elongation at 72° C. for 30 sec were conducted and that finally, reaction was completed at 72° C. for 5 min.

Next, the obtained amplified product was subcloned into pGEM-T Easy vector and then it was subjected to base sequencing by using BigDye Terminator Cycle Sequencing Kit Ver. 3.1 and ABI 3130×1 DNA sequencer.

Next, by referring to the obtained base sequence, BCL11_{—}3′ RACE_F1 primer was designed; and as for 3′RACE, a primer pair of the aforementioned primer and GeneRacer_{—}3′_Primer was used to perform PCR. Note that the composition of the PCR reaction solution (50 μl) is as follows: 1× Blend Taq buffer; dNTP mix 10 nmol; GeneRacer_{—}3′_Primer 10 pmol; BCL11_{—}3′RACE_F1 10 pmol; the 10-fold diluted full-length cDNA solution 1 μl; and Blend Taq DNA polymerase 1.25 U. The reaction conditions of PCR were also such that the annealing temperature was set at 60° C. and the elongation reaction was set at for 1 min, and PCR was performed in the same manner as that described above. The obtained amplified product was subjected to base sequencing. Primers BCL11_{—}5′End_F and BCL11_{—}3′End_R were designed, which were specific to the 5′- and 3′-terminal sequences revealed by the 5′- and 3′-RACE. These were made a primer pair and it was subjected to PCR using KOD FX Neo DNA polymerase. Note that the composition of PCR reaction solution and the reaction conditions of PCR followed the attached user's manual of the DNA polymerase to perform PCR. The obtained amplified product was then subjected to base sequencing and the full-length cDNA sequence of BCL11 was revealed. The obtained base sequence is shown in SEQ ID NO:35 and the amino acid sequence based on the obtained base sequence is shown in SEQ ID NO:4. The base sequences of the primers used in Example 27 are also shown in Table 40.

### Example 28

### cDNA Cloning of BML11b and BML11c

Plant RNA Isolation Reagent was used to extract a total RNA from the algal thallus of Bryopsis maxima that was stored in RNAlater at −20° C. Thereafter, the mRNA was purified with NucleoTrap mRNA and a full-length cDNA was further prepared with GeneRacer Kit.

By referring to the deduced amino acid sequence of BCL11, a degenerate primer BCL11_like_common_R1 was designed, and a solution of the full-length cDNA derived from Bryopsis maxima as a template was subjected to RACE. First, as for 5′RACE, a primer pair of BCL11_like_common_R1 and GeneRacer_{—}5′_Primer, and Blend Taq DNA polymerase were used to perform PCR. Note that the composition of the PCR reaction solution (50 μl) is as follows: 1× Blend Taq buffer; dNTP mix 10 nmol; GeneRacer_{—}5′ Primer 30 pmol; BCL11_like_common_R1 250 pmol; the 10-fold diluted full-length cDNA solution 1 μl; and Blend Taq DNA polymerase 1.25 U. The PCR reaction conditions were also such that after thermal denaturation at 94° C. for 3 min, 35 cycles of thermal denaturation at 94° C. for 30 sec, annealing at 58° C. for 30 sec, and elongation at 72° C. for 30 sec were conducted and that finally, reaction was completed at 72° C. for 5 min.

Next, the obtained amplified product was subcloned into a pGEM-T Easy vector, and then, it was subjected to base sequencing by using BigDye Terminator Cycle Sequencing Kit Ver. 3.1 and ABI 3130×1 DNA sequencer. Consequently, two types of amplified products were obtained although they have similar but different sequences. Thus, primers BML11b_{—}5′End_F and BML11c_{—}5′End_F, which were specific to the respective 5′-terminal sequences, were designed. Moreover, as for 3′RACE, they were subjected to PCR using a primer pair of BML11b_{—}5′End_F and GeneRacer 3′ Primer or of BML11c 5′End F and GeneRacer 3′_Primer, together with KOD FX Neo DNA polymerase. Note that the composition of PCR reaction solution and the reaction conditions of PCR followed the attached user's manual of the DNA polymerase to perform PCR. The obtained amplified product was then subjected to base sequencing and the full-length cDNA sequences of BCL11b and BCL11c were revealed. The obtained base sequences were shown in SEQ ID NO:36 with respect to BML11b and in SEQ ID NO:37 with respect to BML11c. The obtained amino acid sequences are shown in SEQ ID NO:13 with respect to BML11b and in SEQ ID NO:14 with respect to BML11c. The base sequences of the primers used in Example 28 are further shown in Table 40.

### Example 29

### Purification of CBA

One kilogram by wet weight of Codium barbatum was first frozen in liquid nitrogen, powdered, and stirred overnight by adding 500 ml of a buffer of 20 mM Tris-HCl and 150 mM NaCl (TBS, pH 7.5). The mixture was centrifuged at 13,500 g for 30 min and a supernatant was recovered. After addition of ammonium sulfate to bring it to saturation at a final concentration of 75% and stirring for 30 min, it was allowed to stand overnight and was then centrifuged at 13,500 g for 30 min to recover precipitates. The precipitates were dissolved in a small amount of TBS and dialyzed with TBS to eliminate ammonium sulfate. After the dialysate was centrifuged at 10,000 g for 30 min to remove the precipitates, it was dialyzed against a buffer of 20 mM Tris-HCl and 1 M (NH₄)₂SO₄ (pH 7.5), passed through a 3.31 ml of TSKgel Phenyl-5PW column (7.5×75 mm) and eluted with a gradient of 1-0 M (NH₄)₂SO₄ at a flow rate of 0.5 ml/min. Next, fractions having hemagglutination activity were collected and dialyzed against a buffer of 20 mM Tris-HCl and 0.85% NaCl (pH 7.5), whereby 8 mg of purified CBA was obtained from 1 kg of Codium barbatum.

Note that the purified CBA was detected as single bands, respectively, between molecular weights of 6.5 kDa and 14.3 kDa in reductive SDS-PAGE and between molecular weights of 14.3 kDa and 20.1 kDa in non-reductive SDS-PAGE. The purified CBA had an activity of agglutinating trypsin-treated rabbit red blood cells at 781 ng/ml, and had its hemagglutination activity inhibited by pig asialo-thyroglobulin at 31 μg/ml.

### Example 30

### cDNA Cloning of CBA

Plant RNA Isolation Reagent was used to extract a total RNA from the algal thallus of Codium barbatum that was stored in RNAlater at −20° C. Thereafter, the mRNA was purified with NucleoTrap mRNA and a full-length cDNA was further prepared with GeneRacer Kit. CBA purified in Example 29 was used to determine its N-terminal amino acid sequence. Further, based on this N-terminal amino acid sequence, CBA_d_F1 primer was designed and a solution of the full-length cDNA derived from Codium barbatum as a template was subjected to RACE. First, as for 3′RACE, a primer pair of CBA_d_F1 and GeneRacer_{—}3′_Primer, and Blend Taq DNA polymerase were used to perform PCR.

Note that the composition of the PCR reaction solution (10 μl) is as follows: 1× Blend Taq buffer; dNTP mix 2 nmol; GeneRacer_{—}3′ Primer 6 pmol; CBA_d_F1 50 pmol; the 10-fold diluted full-length cDNA solution 1 μl; and Blend Taq DNA polymerase 0.25 U. The reaction conditions of PCR were also such that after thermal denaturation at 94° C. for 5 min, 35 cycles of thermal denaturation at 94° C. for 30 sec, annealing at 50, 52, 54, 56, 58, 60, 62, or 64° C. for 30 sec, and elongation at 72° C. for 90 sec were conducted and that finally, reaction was completed at 72° C. for 5 min. The PCR reaction solution after reaction was recovered and all PCR products from eight temperatures used in the annealing were pooled.

The pooled PCR product was diluted 100-folds with sterilized water to be a template, and nested PCR was performed using as a primer pair, CBA_d_F21 primer (a concentration of 50 pmol) that was designed from a peptide sequence obtained by partial digestion of CFA with Lys-C and GeneRacer_{—}3′_Nested_Primer (a concentration of 2 pmol). Note that the PCR was performed in the same manner as that described above, except that the annealing temperature was set at 54° C.

Next, the obtained amplified product was subjected to base sequencing, and a primer for 5′RACE, CBA_R1, was designed. Further, as for 5′RACE, a primer pair of CBA_R1 and GeneRacer_{—}5′_Primer was used and subjected to PCR using KOD Plus Neo DNA polymerase. The composition of PCR reaction solution followed the attached user's manual of the DNA polymerase. The reaction conditions of PCR were such that after thermal denaturation at 94° C. for 2 min, 35 cycles of thermal denaturation at 98° C. for 10 sec, annealing at 50, 52, 54, 56, 58, 60, 62, or 64° C. for 30 sec, and elongation at 68° C. for 30 sec were conducted and that finally, reaction was completed at 68° C. for 5 min. The PCR reaction solution after reaction was recovered and all PCR products from eight temperatures used in the annealing were pooled. The pooled PCR product was diluted 100-folds with sterilized water to be a template, and nested PCR was performed using as the primer pair, CBA_d_R2 primer and GeneRacer_{—}5′_Nested_Primer. Note that the PCR was performed in the same manner as that described above. The obtained amplified product was subjected to base sequencing, and when combined with the sequence obtained from 3′RACE, the full-length cDNA sequence of CBA was revealed. The obtained base sequence is shown in SEQ ID NO:39. Also, the amino acid sequence based on the obtained base sequence is shown in SEQ ID NO:38. Further, the base sequences of the primers used in Example 30 are shown in Table 40.

### Example 31

### cDNA Cloning of BCL11d

The full-length cDNA derived from Bryopsis corticulans that was prepared in Example 27, as a template, was subjected to PCR using a primer pair of BML11c_{—}5′End_F and GeneRacer_{—}5′_Primer together with KOD Plus Neo DNA polymerase. Note that the composition of the PCR reaction solution and the reaction conditions of PCR followed the attached user's manual of the DNA polymerase. When the obtained amplified product was subjected to base sequencing, there were obtained a sequence that matched BML11c in sequence and a BCL11c-like cDNA that was different from BCL11c in sequence. Therefore, the former was designated BCL11c and the latter was designated BCL11d. To confirm the 5′ terminal sequence of BCL11d, it was subjected to PCR using a primer pair of BCL11c_{—}3′End_R, which was designed by reference to the 3′ terminal sequence of BCL11d and GeneRacer 5′_Primer, together with KOD Plus Neo DNA polymerase. Note that the composition of the PCR reaction solution and the reaction conditions of PCR followed the attached user's manual of the DNA polymerase to perform PCR. The obtained amplified product was subjected to base sequencing, and the full-length cDNA sequence of BCL11d was revealed. The obtained base sequence is shown in SEQ ID NO:41. Also, the amino acid sequence based on the obtained base sequence is shown in SEQ ID NO:40. Further, the base sequences of the primers used in Example 31 are shown in Table 40.

### Example 32

### cDNA Cloning of CFA (CFA1 and CFA2)

Plant RNA Isolation Reagent was used to extract a total RNA from the algal thallus of Codium fragile that was stored in RNAlater at −20° C. Thereafter, the mRNA was purified with NucleoTrap mRNA and a full-length cDNA was further prepared with GeneRacer Kit.

Based on a partial amino acid sequence of CFA, CFA_{—}5′RACE_R1 was designed, and a solution of the full-length cDNA derived from Codium fragile, as a template, was subjected to RACE. First, as for 5′RACE, PCR was performed by using a primer pair of CFA_{—}5′RACE_R1 and GeneRacer_{—}5′_Primer, together with Blend Taq DNA polymerase. Note that the composition of the PCR reaction solution (10 μl) is as follows: 1× Blend Taq buffer; dNTP mix 2 nmol; GeneRacer_{—}5′_Primer 6 pmol; CFA_{—}5′RACE_R1 50 pmol; the 10-fold diluted full-length cDNA solution 1 μl; and Blend Taq DNA polymerase 0.25 U. Also, the reaction conditions of PCR were such that after thermal denaturation at 94° C. for 3 min, 30 cycles of thermal denaturation at 94° C. for 30 sec, annealing at 50° C. for 30 sec, and elongation at 72° C. for 60 sec were conducted and that finally, reaction was completed at 72° C. for 5 min.

Next, PCR was performed by using a primer pair of CFA_{—}5′RACE_R2 designed from the partial amino acid and GeneRacer_{—}5′_Nested Primer, and the template of the previously amplified PCR product. Note that the composition of the PCR reaction solution (50 μl) is as follows: 1× Blend Taq buffer; dNTP mix 2 nmol; GeneRacer_{—}5′_Primer 2 pmol; CFA_{—}5′RACE_R1 50 pmol; the 10-fold diluted full-length cDNA solution 1 μl; and Blend Taq DNA polymerase 0.25 U. Also, the reaction conditions of PCR were such that the annealing temperature was set at 60° C. and the elongation was set at for 1 min for PCR to be performed in the same manner as that described above. When the obtained amplified product was subjected to base sequencing, there were obtained two amplified sequences having different sequences, which were designated CFA1 and CFA2. Next, as for 3′RACE, a primer pair of CFA1_{—}3′RACE_F or CFA2_{—}3′RACE_F and GeneRacer_{—}5′_Primer was used to perform PCR in the same manner as that described above. The obtained amplified product was subjected to base sequencing. Further, when combined with the sequence obtained by the 5′RACE, the full-length cDNA sequences of CFA1 and CFA 2 were revealed. The obtained base sequences are shown in SEQ ID NO:43 for CFA1 and in SEQ ID NO:45 for CFA2. Also, the amino acid sequences based on the obtained base sequences are shown in SEQ ID NO:42 for CFA1 and in SEQ ID NO:44 for CFA2. Further, the base sequences of the primers used in Example 32 are shown in Table 40.

### Example 33

### cDNA Cloning of CLA

Plant RNA Isolation Reagent was used to extract a total RNA from the algal thallus of Codium latum that was stored in RNAlater at −20° C. Thereafter, the mRNA was purified with NucleoTrap mRNA and a full-length cDNA was further prepared with GeneRacer Kit.

Based on the known N-terminal amino acid sequence of CLA, CLA_d_F1 was designed, and a solution of the full-length cDNA derived from Codium latum, as a template, was subjected to RACE. First, as for 3′RACE, PCR was performed by using a primer pair of CLA_d_F1 and GeneRacer_{—}3′ Primer, together with Blend Taq DNA polymerase. Note that the composition of the PCR reaction solution (10 μl) is as follows: 1× Blend Taq buffer; dNTP mix 2 nmol; GeneRacer_{—}3′_Primer 6 pmol; CLA_d_F1 50 pmol; the 10-fold diluted full-length cDNA solution 1 μl; and Blend Taq DNA polymerase 0.25 U. Also, the reaction conditions of PCR were such that after thermal denaturation at 94° C. for 5 min, 35 cycles of thermal denaturation at 94° C. for 30 sec, annealing at 50, 52, 54, 56, 58, 60, 62, or 64° C. for 30 sec, and elongation at 72° C. for 60 sec were conducted and that finally, reaction was completed at 72° C. for 5 min. The PCR reaction solution after reaction was recovered and all PCR products from eight temperatures used in the annealing were pooled.

Next, PCR was performed by using a primer pair of CLA_d_F2 designed from the partial amino acid and GeneRacer_{—}3′_Nested Primer, and the template of the previously amplified, pooled PCR product. PCR conditions were the same as those described above, except that the concentration of CLA_d_F2 was set at 50 pmol, the concentration of GeneRacer_{—}3′_Nested Primer was set at 2 pmol, and the annealing temperature was set at 58° C. The obtained amplified product was next subjected to base sequencing. Further, as for 5′RACE, a primer pair of CLA_{—}3′End_R, which was designed from the sequence information of 3′RACE, and GeneRacer_{—}5′_Primer was used and subjected to PCR using KOD Plus Neo DNA polymerase. The composition of PCR reaction solution followed the attached user's manual of the DNA polymerase. The reaction conditions of PCR were such that after thermal denaturation at 94° C. for 2 min, 35 cycles of thermal denaturation at 98° C. for 10 sec, annealing at 60° C. for 30 sec, and elongation at 68° C. for 30 sec were conducted and that finally, reaction was completed at 68° C. for 5 min. The obtained amplified product was subjected to base sequencing, and when combined with the sequence obtained from 3′RACE, the full-length cDNA sequence of CLA was revealed. The obtained base sequence is shown in SEQ ID NO:47. Also, the amino acid sequence based on the obtained base sequence is shown in SEQ ID NO:46. Further, the base sequences of the primers used in Example 33 are shown in Table 40.

### Example 34

### cDNA Cloning of MPA1 and MPA2

Plant RNA Isolation Reagent was used to extract a total RNA from the algal thallus of Meristotheca papulosa that was stored in RNAlater at −20° C. Thereafter, the mRNA was purified with NucleoTrap mRNA and a full-length cDNA was further prepared with GeneRacer Kit.

By referring to the base sequences of KAA1 described in Example 26, KAA_common_F1 primer was designed, and a solution of the full-length cDNA derived from Meristotheca papulosa, as a template, was subjected to RACE. First, as for 3′RACE, PCR was performed by using a primer pair of KAA_common_F1 and GeneRacer_{—}3′_Primer, together with Blend Taq DNA polymerase.

The PCR reaction solution and reaction conditions of PCR: 8.0 μl of 100×PCR buffer, 8 μl of dNTPmix (2.5 mM each), 4.8 μl of GeneRacer (TM) 3′ Primer, 4 μl of KAA_common_F1 (10 μM), 1.6 μl of the 10-fold diluted solution of the cDNA derived from Meristotheca papulosa, 0.8 μl of Blend Taq (registered trademark) (2.5 U/μl), and sterilized water were added to make 80 μl of reaction solution. After mixing sufficiently, 10 μl each was dispensed and subjected to PCR. The PCR employed T Gradient 96 Thermocycler (manufactured by Biometra GmbH); and after thermal denaturation at 94° C. for 5 min, 30 cycles of thermal denaturation at 94° C. for 30 sec, annealing at 50, 52, 54, 56, 58, 60, 62, or 64° C. for 30 sec, and elongation at 72° C. for 1 min were conducted. Finally, reaction was maintained at 72° C. for 5 min to be completed.

Subsequently, the PCR product for which the annealing was conducted at a temperature of from 50 to 58° C. was pooled and was diluted 100-folds with sterilized water to be a template. Nested PCR was performed by using the template, a primer pair, 4 μl of KAA_{—}3′RACE_d_F1 (100 μM) or KAA_{—}3′RACE_d_F2 (100 μM) and 1.6 μl of GeneRacer_{—}3′_Nested_Primer (10 μM). Note that the PCR was performed in the same manner as that described above.

Next, when the obtained amplified products were subjected to base sequencing, there were obtained DNA fragments that showed sequence homologies to KAA1 in both cases where KAA_{—}3′RACE_d_F1 and GeneRacer 3′ Nested_Primer as well as KAA 3′RACE_d_F1 and GeneRacer 3′_Nested_Primer were used as the primer pairs.

Meanwhile, these have apparently different sequences although similar in sequence; the former was designated MPA1 and the latter was designated MPA2. To confirm the 5′ terminal sequences of MPA1 and MPA2, MPA1_R1 and MPA2_R2 primers were designed by referring to the respective sequences and were subjected to 5′RACE. Specifically, they are subjected to PCR using a primer pair of MPA1_R1 and GeneRacer_{—}5′_Primer or a primer pair of MPA2_R1 and a GeneRacer_{—}5′_Primer, together with Blend Taq DNA polymerase. Note that the composition of PCR reaction solution and the reaction conditions of PCR followed the attached user's manual of the DNA polymerase to perform the PCR. The obtained amplified products were subjected to base sequencing and 5′-terminal base sequences of MPA1 and MPA2 were determined. Further, when combined with the base sequences obtained by the 3′RACE, the full-length cDNA sequences of MPA1 and MPA2 were revealed. The obtained base sequences are shown in SEQ ID NO:49 for MPA1 and in SEQ ID NO:51 for MPA2. Also, the amino acid sequences based on the obtained base sequences are shown in SEQ ID NO:48 for MPA1 and in SEQ ID NO:50 for MPA2. Furthermore, the base sequences of the primers used in Example 34 are shown in Table 40.

### Example 35

### Purification of AC-Avranin

The green alga, Avrainvillea captituliformis (Japanese name unknown) collected in Nha Trang, Vietnam was used as a sample. After collecting, the sample was stored at −30° C. and thawed at 4° C. for use. The thawed sample (300 g) was measured out, shredded with a scissor and powdered with a blender under liquid nitrogen. To this was added 600 ml of a 20 mM Tris-HCl buffer (TB, pH 7.5). After stirring overnight at 4° C., centrifugation was carried out (8,500 rpm, 30 min, and at 4° C.), and a supernatant was obtained to prepare an extract. Next, ammonium sulfate powders were added to the extract under stirring so that a saturated concentration of 75% was attained. After stirring for 30 min, the extract was allowed to stand overnight. This was centrifuged (8,500 rpm, 30 min, and at 4° C.); and the obtained precipitates were dissolved in a small amount of TB and were dialyzed sufficiently against the buffer. Further, an internal solution was recovered to prepare a 75% saturated ammonium sulfate-salted out fraction. The obtained ammonium sulfate-salted out fraction was subjected to gel filtration using Superdex 75 column (10×300 mm; manufactured by GE Healthcare). Specifically, each 1 ml of the ammonium sulfate-salted out fraction was subjected to the Superdex 75 column equilibrated with a 20 mM phosphate buffer containing 0.3 M NaCl (PBS, pH 7.0) and eluted with the buffer. The eluate was fractionated per ml; and the absorbance at 280 nm and the agglutination activity against trypsin-treated rabbit red blood cells were measured. Purification through the gel filtration was carried out 18 times in total. Further, each active fraction was recovered and combined. The purified fractions also displayed a single band at molecular weight of 18,000 Da in reductive SDS-PAGE.

### Example 36

### Purification of algCSA

The specimens of a green alga, Codium subtubulosum that had been cryopreserved at −30° C. after collection were used as a sample. The frozen sample was thawed by allowing to stand overnight at 4° C. on the day before extraction. The thawed sample, 500 g, was shredded with a scissor and was then powdered with a blender under liquid nitrogen. To this was added 1000 ml of a 20 mM Tris-HCl buffer (TB, pH 7.5). After stirring overnight at 4° C., centrifugation was carried out (8,500 rpm, 30 min, and at 4° C.), and a supernatant was obtained to prepare an extract (945 ml, 746 mg of protein). Next, solid ammonium sulfate was added to the extract under stirring so that a saturated concentration of 75% was attained. After further stirring at 4° C. for 30 min, the extract was allowed to stand overnight. This was centrifuged (8,500 rpm, 30 min, and 4° C.); and the obtained precipitates were dissolved in a small amount of TB and were dialyzed sufficiently against TB. Next, an internal solution was recovered to prepare a 75% saturated ammonium sulfate-salted out fraction (117 ml, 242 mg), which was cryopreserved at −20° C. until being provided for the next experiment. Further, the ammonium sulfate-salted out fraction was added to a bovine submaxillary mucin-immobilized column (1×10 cm) equilibrated with 20 mM TB (pH 7.5). After washing the column with the same buffer, the column was eluted sequentially with 1 M NaCl and 0.2 M N-acethyl-D-galactosamine both in the buffer. The flow rate was set at 0.2 ml/min and the eluate was fractionated per 2 ml; and for each fraction, the absorbance at 280 nm and the agglutination activity against trypsin-treated rabbit red blood cells were measured. Further, a 0.2 M GalNAc-eluted fraction (4.5 ml, 1.85 mg) displaying the agglutination activity was made the final, purified authentic sample. In addition, the molecular weight of the purified protein was 13,000 Da.

### INDUSTRIAL APPLICABILITY

As has been described above, according to the present invention, it is possible to distinguish between species within the genus Staphylococcus even at the stationary phase or at the logarithmic growth phase, further in foods. Furthermore, in the present invention it is possible to distinguish not only between species within the genus Staphylococcus, but also between Staphylococcus and bacteria other than the genus Staphylococcus by using HAA, HPA, LEL, STL, Tachylectin-2, ULL, or BCL11. Accordingly, the present invention is useful for the food hygiene inspection or the examination of patients having infection.

### Sequence Listing Free Text

### SEQ ID NOs:18-33, 74-89

<223> Artificially synthesized primer sequence

### SEQ ID NOs:22, 23, 24, 27, 31, 74, 75, 79, 80, 83, 84 and 87

<223> n represents inosine.

### SEQ ID NO:73

<223> Xaa stands pyroglutamic acid.

1. A method for distinguishing between species within the genus Staphylococcus based on binding affinity to at least one lectin as an indicator, the lectin being selected from the group consisting of algMPL, Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, and algCSA.

2. An agent for distinguishing between species within the genus Staphylococcus, comprising at least one lectin selected from the group consisting of algMPL, Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, and algCSA.

3. A kit for distinguishing between species within the genus Staphylococcus, comprising:
a substrate where there is immobilized at least one lectin selected from the group consisting of algMPL, Tachylectin-2, LEL, KAA1, BCL11, CBA, HAA, HPA, STL, proBCA1, proBCA2, ULL, DSA, PWM, UDA, WFL, hypninA3, Tachylectin-3, OAA, PNA, TL, ACG, AC-avranin, MOA, API 144, CV-N, PMA, GSL-II, Garlic lectin, PAA, UEA-II, RSL, CPA, CHA-1, LAA, SHA, LPA, DBA, TPL-1, BML11b, BML11c, PVL, LBA, UPL-1, BPL, CFA1, CFA2, BanLec, BCL11d, FVE, CLA, Pro-CFA I, Pro-CFA II, MPA1, MPA2, and algCSA; and
at least one reagent selected from the group consisting of:
(a) a reagent for detecting a specimen;
(b) a blocking reagent;
(c) a reagent for immobilizing the specimen; and
(d) a reagent for diluting the specimen.

4. At least one lectin selected from the group consisting of (a) to (c) below:
(a) a lectin comprising the amino acid sequence as set forth in SEQ ID NO:3;
(b) a lectin comprising an amino acid sequence having a homology of 90% or more to the amino acid sequence as set forth in SEQ ID NO:3; and
(c) a lectin encoded by a DNA that hybridizes with a DNA comprising the base sequence as set forth in SEQ ID NO:34 under stringent conditions.

5. At least one lectin selected from the group consisting of (a) to (c) below:
(a) a lectin comprising the amino acid sequence as set forth in SEQ ID NO:4;
(b) a lectin comprising an amino acid sequence having a homology of 90% or more to the amino acid sequence as set forth in SEQ ID NO:4; and
(c) a lectin encoded by a DNA that hybridizes with a DNA comprising the base sequence as set forth in SEQ ID NO:35 under stringent conditions.

6. At least one lectin selected from the group consisting of (a) to (c) below:
(a) a lectin comprising the amino acid sequence as set forth in SEQ ID NO: 13;
(b) a lectin comprising an amino acid sequence having a homology of 90% or more to the amino acid sequence as set forth in SEQ ID NO:13; and
(c) a lectin encoded by a DNA that hybridizes with a DNA comprising the base sequence as set forth in SEQ ID NO:36 under stringent conditions.

7. At least one lectin selected from the group consisting of (a) to (c) below:
(a) a lectin comprising the amino acid sequence as set forth in SEQ ID NO: 14;
(b) a lectin comprising an amino acid sequence having a homology of 90% or more to the amino acid sequence as set forth in SEQ ID:14; and
(c) a lectin encoded by a DNA that hybridizes with a DNA comprising the base sequence as set forth in SEQ ID NO:37 under stringent conditions.

8. At least one lectin selected from the group consisting of (a) to (c) below:
(a) a lectin comprising the amino acid sequence as set forth in SEQ ID NO:38;
(b) a lectin comprising an amino acid sequence having a homology of 90% or more to the amino acid sequence as set forth in SEQ ID NO:38; and
(c) a lectin encoded by a DNA that hybridizes with a DNA comprising the base sequence as set forth in SEQ ID NO:39 under stringent conditions.

9. At least one lectin selected from the group consisting of (a) to (c) below:
(a) a lectin comprising the amino acid sequence as set forth in SEQ ID NO:40;
(b) a lectin comprising an amino acid sequence having a homology of 90% or more to the amino acid sequence as set forth in SEQ ID NO:40; and
(c) a lectin encoded by a DNA that hybridizes with a DNA comprising the base sequence as set forth in SEQ ID NO:41 under stringent conditions.

10. At least one lectin selected from the group consisting of (a) to (c) below:
(a) a lectin comprising the amino acid sequence as set forth in SEQ ID NO:42;
(b) a lectin comprising an amino acid sequence having a homology of 90% or more to the amino acid sequence as set forth in SEQ ID NO:42; and
(c) a lectin encoded by a DNA that hybridizes with a DNA comprising the base sequence as set forth in SEQ ID NO:43 under stringent conditions.

11. At least one lectin selected from the group consisting of (a) to (c) below:
(a) a lectin comprising the amino acid sequence as set forth in SEQ ID NO:44;
(b) a lectin comprising an amino acid sequence having a homology of 90% or more to the amino acid sequence as set forth in SEQ ID NO:44; and
(c) a lectin encoded by a DNA that hybridizes with a DNA comprising the base sequence as set forth in SEQ ID NO:45 under stringent conditions.

12. At least one lectin selected from the group consisting of (a) to (c) below:
(a) a lectin comprising the amino acid sequence as set forth in SEQ ID NO:46;
(b) a lectin comprising an amino acid sequence having a homology of 90% or more to the amino acid sequence as set forth in SEQ ID NO:46; and
(c) a lectin encoded by a DNA that hybridizes with a DNA comprising the base sequence as set forth in SEQ ID NO:47 under stringent conditions.

13. At least one lectin selected from the group consisting of (a) to (c) below:
(a) a lectin comprising the amino acid sequence as set forth in SEQ ID NO:48;
(b) a lectin comprising an amino acid sequence having a homology of 90% or more to the amino acid sequence as set forth in SEQ ID NO:48; and
(c) a lectin encoded by a DNA that hybridizes with a DNA comprising the base sequence as set forth in SEQ ID NO:49 under stringent conditions.

14. At least one lectin selected from the group consisting of (a) to (c) below:
(a) a lectin comprising the amino acid sequence as set forth in SEQ ID NO:50;
(b) a lectin comprising an amino acid sequence having a homology of 90% or more to the amino acid sequence as set forth in SEQ ID NO:50; and
(c) a lectin encoded by a DNA that hybridizes with a DNA comprising the base sequence as set forth in SEQ ID NO:51 under stringent conditions.

15. A lectin derived from a green algae (Avrainvillea capituliformis) being present in a fraction obtained by extracting the green alga with a buffer, salting out a obtained soluble fraction, dialyzing a obtained precipitate, and purifying the precipitate through gel filtration, the lectin having a molecular weight of from 15,000 to 20,000 Da as shown in reductive SDS-PAGE and displaying an agglutination activity against trypsin-treated rabbit red blood cells.

16. A lectin derived from a green alga (Codium subtubulosum) being present in a fraction obtained by extracting the green algae with a buffer, salting out a obtained soluble fraction, dialyzing a obtained precipitate, followed by adsorption of the precipitate on a column immobilized with submaxillary mucin and then, elution with N-acetyl-D-galactosamine, the lectin having a molecular weight of from 10,000 to 15,000 Da and displaying an agglutination activity against trypsin-treated rabbit red blood cells.

17. A lectin comprising the lectin according claim 4 and an additional functional protein fused thereto.

18. A DNA encoding the lectin according to claim 4.
